(19) United States
(12) Patent Application Publication
(10) Pub. No.: US 2025/0163239 A1
(43) Pub. Date: May 22, 2025

(54) Metal-Labeled Polymer Microbeads with Control Over Labeling Level

(71) Applicant: The Governing Council of the University of Toronto, Toronto (CA)

(72) Inventors: Mitchell A. WINNIK, Toronto (CA); Jieyi LIU, Burlington (CA)

(73) Assignee: The Governing Council of the University of Toronto, Toronto, ON (CA)

(21) Appl. No.: 18/282,095

(22) PCT Filed: Mar. 15, 2022
(86) PCT No.: PCT/CA2022/050386

§ 371 Date: Apr. 1, 2024

Related U.S. Application Data
(60) Provisional application No. 63/319,608, filed on Mar. 14, 2022.
(60) Provisional application No. 63/161,414, filed on Mar. 15, 2021.

(51) Int. Cl. C08K 3/08

(52) U.S. Cl. CPC C08K 3/08

(57) ABSTRACT

The present disclosure relates to metal labelled polymer microbeads and in particular to lanthanide labelled polymer microbeads for mass cytometry bead based assays and multiplexing applications. The polymer microbead comprises a copolymer comprising a structural monomer and a metal-chelating monomer.

### CROSS-REFERENCE TO RELATED APPLICATIONS

The present disclosure claims the benefit of priority from U.S. patent application No. 63/161,414, filed on Mar. 15, 2021, and from U.S. patent application No. 63/319,608, filed on Mar. 14, 2022, the contents of which are incorporated herein by reference in their entirety.

### FIELD

The present disclosure relates to metal labelled polymer microbeads and in particular to lanthanide labelled polymer microbeads for mass cytometry bead based assays and multiplexing applications.

### INTRODUCTION

Mass Cytometry (MC) is an emerging analytical technique employing an inductively coupled plasma time-of-flight mass spectrometer (ICP-TOF-MS) to analyze the signals of isotopic labels on cell and microbead samples. Bead-based applications in mass cytometry requires one to label the microbeads with various metal ions that can be individually identified by MC.

Bead-based assays are attractive as an analytical technique due to their high sample volume efficiency for multiplex assays with high throughput capacity. A wide variety of applications have been developed using bead-based assay technology, including capture sandwich immunoassays, competitive immunoassays, serology, gene expression profiling and genotyping.^{1,2} In contrast with classical planar or ELISA techniques, bead-based assays employ colloidal suspensions of particles as solid supports for different affinity reagents that can target different molecules as the analytes. By encoding the beads and creating a library of beads as classifiers for the analytes, a variety of captured analytes can be tracked by decoding and identifying individual beads throughout an experiment. In this way, a multi-analyte analysis can be performed simultaneously in a single assay using a set of beads functionalized with different affinity molecules. Most commercially available bead-based assays use luminescence tags as labels to barcode their beads and examine these classifier beads by flow cytometry at high throughput. Luminex has commercialized a library of 500 differently labelled polystyrene microspheres for bead-based assays by flow cytometry with 3 different colors at 10 different levels of intensities, with surface functionality for attaching bioaffinity reagents.³⁻⁵

Mass cytometry (MC) is an emerging multiparameter analytical technique that combines the features of flow cytometry and elemental mass spectrometry to determine the properties of single-cells or bead samples.⁵ In mass cytometry, cell and bead samples are labelled with heavy metal isotopes and individually introduced to the plasma torch of an inductively coupled plasma time-of-flight mass spectrometer (ICP-TOF-MS) to analyze the signals of metal isotope labels. MC is able to accurately detect different metal isotopes based on their atomic masses without channel overlap, and with the low abundance of heavy metal isotopes in biological samples, MC avoids complicated signal compensation processes associated with flow cytometry. It greatly improves the detection of sample features with enhanced resolution and sensitivity compared with fluorescence-based bead assays. Because much larger numbers of heavy metal isotopes can be employed as labels, MC provides a powerful opportunity for high-throughput cytometric bead-based assays with much higher multiplexing capacity.⁷⁻⁸

To develop metal-encoded microbeads for multiplexed MC assays, Abdelrahman et al. introduced the idea of synthesizing a library of microbeads encoded with a variety of metal isotopes at different levels of concentrations that can be individually identified in MC.⁹ The theoretical maximum variability (n) of this microbead library can be calculated with the expression:

$$n = {K}^{M} - 1 \qquad ( 1 )$$

Here, K refers to the levels of metal concentration levels in the microbeads (including a concentration of zero); M is the number of different isotopes encoded in the beads. The term (−1) refers to beads with zero metal content for all the isotopes, which cannot be detected by MC. Lanthanide isotopes are an attractive choice as mass tags to encode PS microbeads because of their similar chemical and physical properties, low natural abundance as well as their high detection sensitivity in MC.⁸ There are 15 lanthanide elements, whose isotopes covers at least 36 detection channels from 139 amu to 176 amu, theoretically available to be employed as bead labels. For example, with 4 levels of concentrations for each encoded isotope, a library of beads with a variety of thousands can in principle be created for MC bead-based assays. To ensure the quality of MC signals, metal containing microbeads employed in MC applications have to be in a size range of around 1˜5 μm in diameter with very narrow size distributions. Smaller microbeads tend to contain fewer metal isotopes which may not be enough to be detected as single events in MC, whereas larger microbeads may not be consistently and fully consumed in the ICP torch.

The synthesis of several types of polymeric microbeads encoded with heavy metal isotopes has been reported. In the approach taken by Abdelrahman et al., lanthanide-encoded polystyrene (PS) microbeads were synthesized by a multistage-dispersion polymerization (DisP) of styrene in the presence of polyvinylpyrrolidone (PVP) as the steric stabilizer and acrylic acid (AA) as the comonomer for metal ion incorporation.⁸ With some optimization, Abdelrahman et al. were able to achieve PS microbeads encoded with ca. 1×10⁸ lanthanide ions per bead that generated strong signal intensities in MC with relatively small bead-to-bead variations (RCV<15%), that fulfill the requirements to use as a reagent in MC applications.^{9,10}

Liang et al. later examined the idea of replacing the comonomer AA with methacrylic acid (MAA) and obtained similar results.¹¹ Other approaches to incorporate metal ions into microbeads through the absorption of styrene-soluble lanthanide chelates into PS beads in seeded emulsion polymerization and covalent incorporation of lanthanide nanoparticles onto polymeric microbeads in dispersion polymerization have also been explored.^{12,13} However, compared with the AA approach developed by Abdelrahman et al., much lower incorporation efficiencies and higher bead-to-bead metal content variations were observed in these later attempts.

The AA approach however is poor for synthesizing a batch of beads with a designed content of different metals for MC calibration purposes. This strategy is poor at controlling the metal incorporation in the bead synthesis, especially when large numbers of different metal ions are mixed with AA in the second stage aliquot. For example, lanthanide ions with smaller ionic radii (e.g. Ho³⁺ and Lu³⁺) tend to incorporate into microbeads much more efficiently than lanthanide ions with larger ionic radii (e.g. Ce³⁺ and Eu³⁺).^{14,15}

This lack of controllability is a time-consuming problem, particularly for example when one seeks to synthesize a plurality or a library of microbeads encoded with various metal ions and defined metal content at different levels for each metal.

Microbeads can be used for various qualitative and quantitative applications for instance for analyte detection. Some cytokines are believed to play a critical role in the pathology of COVID-19. However, there are limitations to current research tools to fully investigate the mechanism of cytokine generation and action.³⁴

Cytokines are soluble signaling protein molecules produced by cells at picomolar to nanomolar concentrations to regulate immune responses and modulate cellular activities. They are an exceptionally large and diverse group of pro- and anti-inflammatory factors in the body.³⁵ Deep characterizations of cytokines in blood samples can provide critical details of immune responses to disease or infections, or to a vaccine, therapy and intervention. Enzyme-linked immunosorbent assays (ELISAs) are the most extensively used monoplex Ab-based immunoassays to analyze cytokines in biological samples. However, ELISA assays require substantial amounts of sample and are time consuming when large numbers of cytokines are analyzed at once.^{36, 37}

Bead-based immunoassays are a technology platform that offers information-rich multiparametric analysis in a high-throughput setting. Bead-based assays employing fluorescent dyes for signal detection are the current gold standard for multiplexed cytokine analysis, and kits for cytokine analysis are commercially available. In this type of assay, polymer microbeads are labeled with 2 or 3 different fluorescent dyes at different concentrations to well-defined intensities. Successful antigen detection is recognized by a reporter antibody (Ab) labeled with dye with a distinct emission.³⁸⁻⁴⁴ A recent report from 6 College of American Pathologists cytokine surveys administered from 2015 to 2018, however, describes variability in cytokine analysis in a set of four cytokines (IL-1, IL-6, IL-8, TNF-α).⁴⁵ This study examined variability between laboratories and variability across method, even within the same laboratory. This report highlights some of the current challenges in quantitative cytokine detection.

### SUMMARY

Described herein are metal-encoded polymer microbeads that can be synthesized by multi-stage dispersion polymerization and comprise a structural monomer and a metal-chelating monomer that comprises a chelator that is chelated to a metal prior to being subjected to polymerization. The microbead comprises, and is formed by polymers including copolymers. As shown in embodiments herein, microbeads obtained have a narrow size distribution and defined amounts of different metals. The quantity of metal incorporated into the microbead is substantially consistent from bead to bead; allowing for substantially uniform populations of microbeads to be obtained. The metal can be incorporated throughout the microbead as opposed to only being present on the surface. Further, as described in some embodiments, the microbeads of the present disclosure can be functionalized on the surface of the microbeads and also conjugated to biomolecules such as antibodies or other affinity reagents.

In particular, provided in certain embodiments are microbeads with substantially controlled metal content and methods for making them. A metal encoded microbead comprises at least one metal element, and may comprising a plurality of metal elements. A metal element may comprise a natural abundance of isotopes of the element or may comprise one or more enriched isotopes of the element. As shown in the Examples, lanthanide-encoded microbeads were made by employing polymerizable metal-complexes as the ligands in a 2-stage dispersion polymerization. In one example, diethylenetriamine pentaacetic acid (DTPA) was functionalized with a polymerizable monomer by reacting DTPA dianhydride with 4-vinylbenzyl amine (VBA). Then, various lanthanide metal-ion complexes of this DTPA derivative were prepared. The metal-encoded microbeads were synthesized by introducing these polymerizable metal-DTPA complexes into dispersion polymerization reactions of monomers such as styrene. The microbeads obtained have very small bead-to-bead variations in their size and metal content. Similar metal incorporation efficiencies were found in bead syntheses with different metal ions complexed to this polymerizable chelator. The metal content in the microbeads prepared using this approach was linearly dependent on the metal complex feed in the bead syntheses regardless the type of metal. Batches of microbeads encoded with four lanthanides at three different concentrations were prepared. These microbeads generated MC signal intensities at three different levels with very good baseline resolution.

As an illustrative example, and as further described herein the surface of a batch of microbeads was surface functionalized and conjugated to an antibody and used to detect a target analyte (e.g. sample biomolecule).

As shown herein as an exemplary application of the microbeads of the present disclosure in mass cytometry (MC), a multiplexed bead-based assay was developed for the detection of analytes including cytokines. In this type of assay, the classifier beads were labeled with heavy metal isotopes at different levels of metal incorporation. Each classifier bead carried a different Ab on its surface. The reporter can be a metal or metal oxide nanoparticle (NP) with an appropriate Ab of other biorecognition element bound to its surface. Samples were injected stochastically into the plasma torch of an inductively coupled plasma time of flight mass spectrometer. The instrument is capable of single mass resolution over the range m/z 85 to m/z 209. Thus, as shown, a very high level of multiplexing was possible. In a bead-based assay by MC, cytokines were used as the exemplary targets.

As described herein, a 4-plex analysis of a mixture of four cytokines is demonstrated. A 9-plex assay was also exemplified. The 9-plex assay was tested on a sample of peripheral blood mononuclear cells (PBMCs), comparing an unstimulated sample with a sample stimulated to promote cytokine secretion.

Accordingly, in one aspect, the present disclosure includes a metal-encoded microbead comprising:
a copolymer comprising:
   a structural monomer, and
   a metal-chelating monomer comprising a metal and a chelator; wherein the chelator coordinates the metal at least at 3 sites; and
   wherein the structural monomer does not comprise the chelator.

In another aspect, the present disclosure includes a population of microbeads of the present disclosure.

In another aspect, the present disclosure includes a kit comprising a plurality of distinct populations of microbeads of the present disclosure.

In another aspect, the present disclosure includes a method of preparing a metal-encoded microbead comprising
polymerizing a structural monomer in the presence of a steric stabilizer in a nucleation stage to obtain a first mixture comprising polymerized structural monomer, unpolymerized structural monomer, and the steric stabilizer;
combining the first mixture with a metal-chelating monomer comprising a metal and a chelator attached to at least one polymerizable end group to obtain a second mixture,
wherein the chelator coordinates the metal at least at 3 sites and wherein the metal-chelating monomer is polymerizable with the structural monomer; and
polymerizing the second mixture to form a copolymer of the microbead;
wherein the structural monomer does not comprise the chelator.

In another aspect, the present disclosure includes a microbead prepared by a method of the present disclosure.

In another aspect, the present disclosure includes a method of preparing metal-encoded microbeads, the method comprising:
providing an aqueous dispersion of swellable seed particles and an anionic surfactant;
   contacting the aqueous dispersion with monomers comprising a structural monomer and a metal-chelating monomer, wherein the metal-chelating monomer comprises a metal and a chelator attached to at least one polymerizable end, and wherein the chelator coordinates the metal at least at 3 sites;
allowing the monomers to diffuse into the seed particles to form an aqueous dispersion of swollen seed particles; and
initiating polymerization of the monomers in the aqueous dispersion of swollen seed particles;
wherein the structural monomer does not comprise the chelator.

### DRAWINGS

Illustrative embodiments of the present disclosure will be further described in relation to the drawings in which:

FIG. 1 is a schematic showing an ¹H-NMR spectrum of Na₃ (DTPA-VBAm₂) molecule dissolved in D₂O. The structure of Na₃ (DTPA-VBAm₂) given has protons labelled corresponding to the represented chemical shifts.

FIG. 2A is an SEM image and 2B is a size graph of metal containing microbeads Ce-1 synthesized in the presence of Ce (DTPA-VBAm₂) metal complexes added in the second stage. (d=2.9 μm, CV=1.2%).

FIG. 3 is a series of graphs depicting the incorporation efficiency of five types of metal ions into Y-1, Ce-1, Eu-1, Ho-1, Lu-1 and 5E1 microbeads (FIG. 3A) and into 4E1, 4E2 and 4E3 microbeads (FIG. 3B) using M (DTPA-VBAm₂) complexes (M=Y, Ce, Eu, Ho, and Lu).

FIG. 4A shows a graph depicting the MC signal intensities and FIG. 4B shows a graph depicting the metal content for 5-element-encoded microbeads (5E1) prepared in presence of M (DTPA-VBAm₂) metal complexes (M=Y, Ce, Eu, Ho, and Lu) in the 2^{nd} stage aliquot. The error bars in a) and b) represent the RSD of MC signal intensities and the SD of metal content evaluated from MC signal intensities.

FIG. 5 is a graph depicting the linear dependence of the metal content concentration in microbeads on the feed concentration of metal complexes in the second stage aliquots. The solid symbols represent data from the preliminary bead syntheses (Y-1, Ce-1, Eu-1, Ho-1, Lu-1, and 5E1). The solid line is the linear regression of these solid data points. The open symbols represent the data from bead syntheses samples 4E1, 4E2, and 4E3, where we used the linear relationship observed in preliminary bead syntheses as a guide for the bead synthesis design.

FIG. 6A-E are a series of graphs depicting the MC signal intensities of three different populations of PS microspheres prepared in presence of M (DTPA-VBAm₂) metal complexes (M=Ce, Eu, Ho, and Lu) in the 2^{nd} stage aliquots, representing the MC signal intensity histograms of ¹⁴⁰Ce, ¹⁵¹Eu, ¹⁵³Eu, ¹⁶⁵Ho, and ¹⁷⁵Lu, respectively. The x-axis in each figure represents the signal intensity of the isotope, and the y-axis represents the number of beads normalized to 100. The first, second and third histograms describe the signal from 4E1, 4E2, and 4E3 microbeads, respectively.

FIG. 7A is a schematic showing an antigen detection agent using M (DTPA-VBAm₂)-encoded microspheres (Eu-1). In this scheme, Eu-encoded microbeads surface functionalized with a goat-anti-mouse-IgG, are incubated with ¹⁷⁵Lu-labeled mouse IgG as the reporter. Washed microbeads are then examined by MC for both ¹⁵³Eu and ¹⁷⁵Lu signals as a proof of reporter detection.

FIG. 7B depicts a histogram of MC measurements generated using the antigen detection agent of FIG. 7A showing the ¹⁷⁵Lu signal intensity for Goat Anti-Mouse (GAM)-modified Eu-1 microbeads (Eu-1/GAM). In a second experiment, NAv-modified Eu-1 microbeads (Eu-1/NAv) without GAM gave the weak signal shown in magenta as a negative control.

FIG. 8A is a ¹H-NMR spectrum of Na₃ (DTPA-BAm₂) and FIG. 8B is a ¹H-NMR spectrum of Na₃ (DTPA-ALAm₂) measured in D₂O. FIG. 8C is a ¹H-NMR spectrum of Na₃ (DTPA-AmPMAm₂). The structure of these molecules given has protons labelled corresponding to the represented chemical shift.

FIG. 9A is a ¹H-NMR (500 MHZ) spectrum of Ce (DTPA-VBAm₂), FIG. 9B is a 1H-NMR (500 MHz) spectrum of Ce (DTPA-BAm₂), FIG. 9C is a ¹H-NMR (500 MHZ) spectrum of Ce (DTPA-ALAm₂), and FIG. 9D is a ¹H-NMR (500 MHz) spectrum of Ce (DTPA-AmPMAm₂) measured in D₂O. The resonance peaks in the figure were broadened and shifted because Ce (III) is a paramagnetic NMR shift reagent.

FIG. 10A is a ¹H-NMR (500 MHz) spectrum of Y (DTPA-VBAm₂), FIG. 10B is a ¹H-NMR (500 MHz) spectrum of Eu (DTPA-VBAm₂), FIG. 10C is a ¹H-NMR (500 MHZ) spectrum of Ho(DTPA-VBAm₂), and FIG. 10D is a ¹H-NMR (500 MHZ) spectrum of Lu(DTPA-VBAm₂) measured in D₂O. The resonance peaks in b) and c) were broadened and shifted because Eu(III) and Ho(III) are paramagnetic NMR shift reagents.

FIG. 11A to D are histograms of MC ¹⁴⁰Ce signal intensity counts in Ce-1, Ce-2, Ce-3, and Ce-4 microbeads respectively: the x-axis is the ¹⁴⁰Ce signal intensity; the y-axis is the number of beads normalized to 100.

FIG. 12A is a histogram of isotope signal intensity counts in microbead samples for Y-1; FIG. 12B is a histogram of isotope signal intensity counts in microbead samples for Eu-1. FIG. 12C is a histogram of isotope signal intensity counts in microbead samples for Ho-1, and FIG. 12D is a histogram of isotope signal intensity counts in microbead samples for Lu-1. The x-axis is the isotope signal intensity; the y-axis is the number of beads normalized to 100.

FIG. 13A is a graph depicting the release profiles of metal ions Ce³⁺ (square), Eu³⁺ (circle), Ho³⁺ (up-pointing triangle) and Lu³⁺ (down-pointing triangle), from 4E3 microbeads (solid symbols and solid lines) at 0.5% solids content into a pH 3.0 buffer solution: 50 mM sodium acetate determined by ICP-MS.

FIG. 13B is a graph depicting the release profiles of metal ions Ce³⁺ (square), Eu³⁺ (circle), Ho³⁺ (up-pointing triangle) and Lu³⁺ (down-pointing triangle), from 4E3 microbeads (solid symbols and solid lines) at 0.5% solids content into a pH 7.0 buffer solution: 10 mM ammonium acetate determined by ICP-MS.

FIG. 13C is a graph depicting the release profiles of metal ions Ce³⁺ (square), Eu³⁺ (circle), Ho³⁺ (up-pointing triangle) and Lu³⁺ (down-pointing triangle), from 4E3 microbeads (solid symbols and solid lines) at 0.5% solids content into a pH 10.5 buffer solution: 200 mM sodium carbonate/bicarbonate determined by ICP-MS.

FIG. 13D is a graph depicting the release profiles of metal ions Ce³⁺ (square), Eu³⁺ (circle), Ho³⁺ (up-pointing triangle) and Lu³⁺ (down-pointing triangle), from 4E3 microbeads (solid symbols and solid lines) at 0.5% solids content into a 1% PVP solution determined by ICP-MS. As a comparison, the open symbols and dash lines represent the release profiles of metal ions from a batch of microbeads prepared by AA approach under the same conditions as 4E3 DTPA-beads.

FIG. 14 is a schematic of a multi-step strategy to functionalize microbead surface with Goat Anti-Mouse (GAM) by silica coating.

FIG. 15 is a schematic of a microbead assay of the present disclosure.

FIG. 16 is a schematic illustrating an exemplary multiplexed bead-based sandwich immunoassay by MC carried out in a 96-well filter plate.

FIG. 17 are dot-plot diagrams of 11 types of classifier microbeads (C1 to C11), where panel A shows the ¹⁴⁰Ce-¹⁴²Ce isotopic dot-plot diagram of a mixture of 11 types of classifier microbeads (C-1 to C-11). The oval circle isolates the singlet events of 11 types of microbeads. Panels b-k are dot-plot-diagrams showing the gating strategies to individually identify C-1 to C-11 microbeads by MC.

FIG. 18 are histograms of the reporter signal intensities on IL-4 classifier beads (C-5) in a series of four-plex assays of standard solutions at various IL-4 concentrations. (a), (b), and (c) AuNP was employed as the reporter in four-plex assays of standard solutions containing IL-4 at concentrations of 0, 1.2, and 20 pg/mL, respectively. (d), (e), and (f) NanoGold was employed as the reporter in four-plex assays of standard solutions containing IL-4 at 0, 1.2, and 20 μg/mL, respectively.

FIG. 19 are standard curves of two sets of four-plex assays for (a) IL-4, (b) IL-6, (c) IFNγ, and (d) TNFα. The x-axis in each plot represents the analyte concentration and the and y-axis represents the median MC signal intensity of NPs attached to the corresponding classifier beads. Two different types of streptavidin-conjugated reporter (AuNP and NanoGold) were investigated in these four-plex assays. The results are presented for the AuNP as circles (●) and for the NanoGold as squares (▪). Negative events with ¹⁹⁷Au signal intensities of ≤1 count per bead were excluded from the statistical analysis for median intensities. The dose-response curves were drawn by fitting the experimental results with a four-parameter logistic regression model.

FIG. 20 shows standard curves of four sets of nine-plex assays for (a) IL-1β, (b) IL-4, (c) IL-6, (d) IL-10, (e) IL-18, (f) IFNγ (g) TNFα, (h) CD163 and (i) CXCL-9 at different concentrations of biotinylated anti-CD163 and anti-CXCL-9 in the detection Ab cocktails. The x-axis in each plot represents the analyte concentration. The y-axis in each plot represents the median MC signal intensity of AuNP attached to the corresponding type of classifier beads. To minimize the background noise at low analyte concentrations, the concentrations of biotinylated anti-CD163 and anti-CXCL9 in the detection Ab cocktails were reduced from 2.5 to 2.0, 1.0 and 0.5 μg/mL, while the concentrations of other detection Abs were kept constant at 2.5 μg/mL in the cocktails. The results of these assays are plotted for Ab concentrations of 2.5 μg/mL with filled circles (●), of 2.0 μg/mL with filled squares (▪), of 1.0 μg/mL with filled triangles (▴), and of 0.5 μg/mL with filled diamonds (♦). Negative events with ¹⁹⁷Au signal intensities of ≤1 count per bead were excluded from the statistical analysis for median intensities. The dose-response curves were drawn by fitting the experimental results with a four-parameter logistic regression model.

FIG. 21 are histograms showing median ¹⁹⁷Au signal intensities of AuNP reporter attached to classifier beads in nine-plex assays for the analysis of (a) IL-1β, (b) IL-4, (c) IL-6, (d) IL-10, (e) IL-18, (f) IFNγ, (g) TNFα, (h) CD163, and (i) CXCL-9 in the stimulated and unstimulated PBMC samples at different sample dilution ratios. Solid columns in the figure represent the assay results of the stimulated samples, while striped columns represent the assay results of the unstimulated samples.

FIG. 22(a) is a SEM image of C-1 microbeads prepared by two-stage DisP. FIG. 22(b) is a graph showing the efficiencies of six types of metal ions incorporated into the C-1 microbeads using M (DTPA-VBAm₂) complexes (M=La, Ce, Pr, Tb, Ho, and Tm). Error bars represent the standard deviation of three measurements on the same solution.

FIG. 23 are standard curves of three sets of four-plex assays for (a) IL-4, (b) IL-6, (c) IFNγ and (d) TNFα at different reporter (NP) concentrations. The x-axis in each plot represents the analyte concentration. The y-axis in each plot represents the median MC signal intensity of AuNP attached to the corresponding classifier beads. Three concentrations of AuNP with 200×, 400×, and 800× dilutions from the stock solution were investigated in these four-plex assays. Their results are presented for dilutions of 200× with circles (●), of 400× with squares (▪), and of 800× with triangles (▴). Events with ¹⁹⁷Au signal intensities≤1 count per bead were excluded from the statistical analysis for median intensities. The dose-response curves were drawn by fitting the experimental results with a four-parameter logistic regression model.

FIG. 24 is a summary chart of median MC signal intensities of AuNP attached to classifier beads in a series of nine-plex assays of blank samples in the absence of analyte molecules (0 pg/mL).

FIG. 25 are standard curves of the nine-plex assays for IL-1β, IL-4, IL-6, IL-10, IL-18, IFNγ, TNFα, CD163 and CXCL-9 using the same assay conditions for the analysis of stimulated and unstimulated PBMC samples. The dose-response curves were drawn by fitting the experimental results with a four-parameter logistic regression model.

FIG. 26 are graphs showing cytokine concentrations in the stimulated and unstimulated PBMC samples calculated based on the dose-response standard curves in FIG. 25. Some measured MC intensity values presented in FIG. 21 are lower than the minimum values of the 4P-LR modeled standard curves presented in FIG. 25. No concentration is calculated from these values.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the disclosure exist as individual salts and hydrates, as well as a combination of, for example, a solvate of a salt of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. For example, a metal chelated to a second component can be different from a metal chelated to a first component, when the second component and the first component can have the same chelator. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or condition would depend on the specific synthetic manipulation to be performed, the identity of the molecule(s) to be transformed and/or the specific use for the compound, but the selection would be well within the skill of a person trained in the art.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form, as well as mixtures thereof, are included within the scope of the present disclosure.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least or up to +5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cn1-n2”. For example, the term C1-10alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cn1-n2”. For example, the term C2-6alkylene means an alkylene group having 2, 3, 4, 5 or 6 carbon atoms.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen or C1-6alkyl.

The term “cycloalkyl,” as used herein, whether it is used alone or as part of another group, means a saturated carbocyclic group containing one or more rings. The number of carbon atoms that are possible in the referenced cycloalkyl group are indicated by the numerical prefix “Cn1-n2”. For example, the term C3-10cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring. In an embodiment of the disclosure, the aryl group contains from 6, 9 or 10 carbon atoms, such as phenyl, indanyl or naphthyl.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix Cn1-n2 this prefix indicates the number of carbon atoms in the corresponding carbocyclic group, in which one or more, suitably 1 to 5, of the ring atoms is replaced with a heteroatom as defined above.

The term “heteroaryl” as used herein, whether it is used alone or as part of another group, refers to cyclic groups containing at least one heteroaromatic ring in which one or more of the atoms are a heteroatom selected from O, S and N. When a heteroaryl group contains the prefix Cn1-n2 this prefix indicates the number of carbon atoms in the corresponding carbocyclic group, in which one or more, suitably 1 to 5, of the ring atoms is replaced with a heteroatom as defined above.

All cyclic groups, including aryl and cyclo a groups, contain one or more than one ring (i.e. are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “atm” as used herein refers to atmosphere.

The term “MS” as used herein refers to mass spectrometry.

The term “aq.” as used herein refers to aqueous.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

It can be appreciated that in the formation of a copolymer, certain polymerizable end groups of a metal-chelating monomer are better matched or preferentially polymerize with certain structural monomers. Reactivity of comonomers can be determined using methods known in the art through for example determination of reactivity ratio. For example, vinyl and methylvinyl react with vinyl ethers, arylvinyl reacts with other styrenes and acrylates react with other acrylates.

The term “EDTA” as used herein refers to ethylenediaminetetraacetic acid.

The term “DTPA” as used herein refers to diethylenetriaminepentaacetic acid.

The term “EGTA” as used herein refers to egtazic acid.

The term “EDDS” as used herein refers to ethylenediamine-N, N′-disuccinic acid.

The term “EDDHA” as used herein refers o ethylenediamine-N,N′-bis(2-hydroxyphenylacetic acid).

The term “BAPTA” as used herein refers to 1,2-bis(o-aminophenoxy) ethane-N,N,N′,N′-tetraacetic acid.

The term “TACN” as used herein refers to 1,4,7-triazacyclononane.

The term “TACD” as used herein refers to 1,5,9-triazacyclododecane.

The term “cyclen” as used herein refers to 1,4,7,10-tetraazacyclododecane.

The term “cyclam” as used herein refers to 1,4,8,11-tetraazacyclotetradecane.

The term “(13) aneN4” as used herein refers to 1,4,7,10-tetrazacyclotridecane.

The term “1,7-diaza-12-crown-4” as used herein refers to 1,7-dioxa-4,10-diazacyclododecane.

The term “1,10-diaza-18-crown-6” as used herein refers to 1,4,10,13-tetraoxa-7,16-diazacyclooctadecane.

The term “DFO” as used herein refers to desferrioxamine.

The term “TACD-type chelator”, “TACN-type chelator”, “cyclen-type chelator” or the like as used herein refers to a chelator that comprises a specified base structure (i.e. TACD, TACN, cyclen, etc.) where the specified base structure can be further substituted at available hydrogen atoms.

The term “swellable polymer seed” as used herein refers to a polymer particle that is capable to increasing in volume. For example, a swellable polymer seed can increase in volume when contacted with a swelling agent. The swelling agent can be for example an anionic surfactant and/or an organic compound. Once a swellable polymer seed is swollen, compounds such as monomers (e.g. structural monomer and metal-chelating monomer), steric stabilizers, and polymerization initiators can diffuse into the interior of the swollen polymer seed. Subsequent polymerization of the monomers can occur.

The term “substantially oxygen-free conditions” as used herein refers to reaction conditions wherein the oxygen content is low or non-existent. For example, substantially oxygen-free conditions can refer to a reaction condition where the reaction is carried out under an inert atmosphere, for example a noble gas (e.g. helium, argon) or nitrogen atmosphere. For example, substantially oxygen-free conditions can refer to an oxygen content between about 0 ppm to about 5 ppm, about 0 ppm to about 3 ppm, about 0 ppm to about 2 ppm, or about 0 ppm to about 1 ppm, or about 0.01 ppm to about 2 ppm.

The term “antibody” as used herein is intended to include monoclonal antibodies, polyclonal antibodies, and chimeric antibodies and binding fragments thereof. The antibody may be from recombinant sources and/or produced in transgenic animals. Antibodies can be fragmented using conventional techniques. For example, F(ab′) 2 fragments can be generated by treating the antibody with pepsin. The resulting F(ab′) 2 fragment can be treated to reduce disulfide bridges to produce Fab′ fragments. Papain digestion can lead to the formation of Fab fragments. Fab, Fab′ and F(ab′) 2, scFv, dsFv, ds-scFv, dimers, minibodies, diabodies, bispecific antibody fragments and other fragments can also be synthesized by recombinant techniques. Antibody fragments as used herein mean binding fragments

The term “oligonucleotide” as used herein refers to a nucleic acid comprising, a sequence of nucleotide or nucleoside monomers consisting of naturally and non-naturally occurring bases, sugars, and intersugar (backbone) linkages, and includes single-stranded and double-stranded molecules, RNA and DNA. Oligonucleotides may be long (e.g. greater than 1000 monomers and up to 10K monomers), medium sized (e.g. between and inclusive of 200 and 1000 nucleotides) or short for example less than 200 monomers, 100 monomers, 50 monomers, including non-naturally occurring monomers. The term “oligonucleotide” includes, for example, single stranded DNA (ssDNA), genomic DNA (gDNA), complementary DNA (cDNA, reverse transcribed from an RNA), messenger RNA (mRNA), “antisense oligonucleotides” and “miRNA” as well as oligonucleotide analogues such as “morpholino oligonucleotides”, “phosphorothioate oligonucleotides”, or any oligonucleotide or analog thereof known to one of skill in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

For ranges described herein, subranges are also contemplated, for example every, 0.1 increment there between. For example, if the range is 0 ppm to about 5 ppm, also contemplated are 0.1 ppm to about 5 ppm, 0 ppm to about 4.9 ppm, 0.1 ppm to about 4.9 ppm and the like.

### II. Compounds, Compositions and Kits of the Present Disclosure

Accordingly, in one aspect, the present disclosure includes a metal-encoded microbead comprising:
a copolymer comprising:
   a structural monomer, and
   a metal-chelating monomer comprising a metal and a chelator;
wherein the chelator coordinates the metal at least at 3 sites, and wherein the structural monomer does not comprise the chelator.

The microbead of the present disclosure can comprise substantially uniformly distributed metal.

For example, the metal chelated to the metal-chelating monomer is not limited to being surface bound but rather can be found distributed throughout and/or in an interior portion of the microbead of the present disclosure.

In some embodiments, the chelator coordinates the metal at least at 4 sites, at least at 5 sites, at least at 6 sites, at least at 7 sites, or at least at 8 sites. For example, DTPA is an octadentate ligand (capable of coordination at 8 sites).

In some embodiments, the structural monomer does not comprise any chelator (e.g., does not comprising any chelator that coordinates a metal at least at 2 sites).

In some embodiments, the structural monomer is metal-free. For example, the structural monomer may not comprise a metal through chelation, through a covalent bond (such as a Tellurium in a carbon backbone of the structural monomer), or optionally through any other means. The structural monomer may not comprise a transition metal or a class of transition metals. For example, the structural monomer may not comprise a rare earth metal (such as a lanthanide) and/or soft metal as described herein. In some embodiments, such as in an application for mass cytometry, metal-chelating monomer may comprise a heavy metal (e.g., of 80 amu or greater), while the structural monomer does not comprise a heavy metal (e.g., of 80 amu or greater).

In some embodiments, the structural monomer is selected from substituted or unsubstituted styrene, alpha-methylstyrene, acrylic acid and esters and amides thereof, methacrylic acid and esters and amides thereof, and derivatives thereof. In one embodiment, the structural monomer is selected from substituted or unsubstituted styrene and/or combinations thereof.

In some embodiments, the metal-chelating monomer has a structure of Formula I prior to polymerization

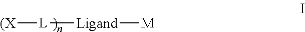

wherein Ligand is the chelator, L is a linker, X is a polymerizable end group, M is the metal, and n is 1 or an integer greater than 1, wherein the metal-chelating monomer is neutral in charge prior to polymerization.

It can be appreciated that the metal is chelated to the chelator of the metal-chelating monomer through ionic, non-covalent interactions. As such, metal is incorporated into the microbead of the present disclosure through non-covalent interactions.

In some embodiments, L is selected from a bond, C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O) O, or mixtures thereof. Each of the alkylene, aryl, alkylaryl, alkylheteroaryl, cycloalkyl, cycloalkylaryl, and cycloalkylheteroaryl can be independently unsubstituted or substituted with one or more substituents which can be selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

The L can for example, be attached to the chelator through an amide or an ester group/linkage.

It can be appreciated that when the structural monomer and the metal-chelating monomer have similar hydrophobicity, the incorporation or mixing of one into the other is favoured. For example, when the structural monomer is hydrophobic, a hydrophobic metal-chelating monomer can have more favourable interactions with the structural monomer, leading to more efficient mixing of the two monomers. Thus, in some embodiments, L can be hydrophobic.

In some embodiments, the polymerizable end group is selected from arylvinyl, styrene, alpha-methylstyrene, acrylate ester, methacrylate ester, acrylamide, 2-methylacrylamide, and mixtures thereof, optionally the polymerizable end group is arylvinyl or styrene. In some embodiments, the polymerizable end group is arylvinyl or vinyl ester.

The chelator can for example be tridentate. In some embodiments, the chelator is tetradentate, pentadentate, hexadentate, heptadentate, or octadentate, optionally the chelator is hexadentate or octadentate.

In some embodiments, the chelator comprises an aminopolyacid moiety, or a derivative thereof. In some embodiments, the derivative of the aminopolyacid moiety includes amides of the aminopolyacid moiety.

For example, the aminopolyacid moiety can be selected from aminopolycarboxylic acid, aminopolyphosphonic acid, or combinations thereof.

In some embodiments, L is a bond, the chelator is an aminopolyacid, and the polymerizable end group is arylvinyl. For example, the metal-chelating monomer can be vinylbenzene iminodiacetic acid coordinated to the metal or divinylbenzene iminodiacetic acid coordinated to the metal.

In other embodiments, the aminopolyacid moiety is a substituted oligomer of one or more of ethylene imine, propylene amine, or mixtures thereof, the oligomer being substituted with two or more carboxylic acids and/or phosphonic acids. The oligomer can be a crown ether or an aza-crown ether. In some embodiments, the aminopolyacid moiety is a substituted oligomer of one or more of ethylene oxide, ethylene imine, propylene oxide, propylene amine, ethanolamine, propanolamine, aminophenol cyclohexane diamine, or mixtures thereof.

In yet other embodiments, the oligomer is further substituted with one or more substituents selected from C1-C6 alkyl, C1-C6 alkenyl, C3-C8 cycloalkyl, C3-C8 heterocycloalkyl, halo, alcohol, amine, amide, ester, aryl, heteroaryl, akylaryl, alkylheteroaryl, C3-C8 cycloalkylaryl, C3-C8 cycloalkylheteroaryl, CN, or mixtures thereof.

In some embodiments, the chelator is selected from DFO, EDTA, DTPA, EGTA, EDDS, EDDHA, BAPTA, H4neunpa, H6phospa, H4CHXoctapa, H4octapa, H2CHXdedpa, H5decapa, Cy-DTPA, Ph-DTPA, a TACN-type chelator, a TACD-type chelator, a cyclen-type chelator, a cyclam-type chelator, a (13) aneN4-type chelator, a 1,7-diaza-12-crown-4-type chelator, a 1,10-diaza-18-crown-6-type chelator, or derivatives thereof.

For example, the TACN-type chelator can be NOTA, NOPO, TRAP, or derivatives of any thereof.

In some embodiments, the cyclen-type chelator is DOTA or derivatives thereof.

In some embodiments, the cyclam-type chelator is selected from TETA, cross bridged-TETA, DiAmSar, or derivatives thereof.

In other embodiments, the (13) aneN4-type chelator is selected from TRITA or derivatives thereof.

In yet other embodiments, the 1,10-diaza-18-crown-6-type chelator is selected from MACROPA, or derivatives thereof.

In some embodiments, the chelator is selected from DTPA, Cy-DTPA, Ph-DTPA, or derivatives thereof.

For example, the derivative of DTPA can comprise DTPA having two adjacent carbon atoms joined together with atoms therebetween to form a 5-membered or 6-membered ring, optionally a cycloalkyl ring, an aryl or a heteroaryl ring.

Prior to polymerization, the monomers described herein are unreacted, i.e. the monomers comprise polymerizable end groups that can participate in polymerization. In some embodiments, prior to polymerization, the metal-chelating monomer is

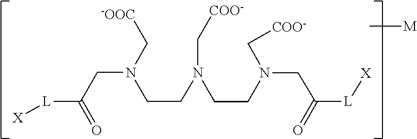

wherein L and X are as described herein.

In other embodiments, prior to polymerization, the metal-chelating monomer is selected from

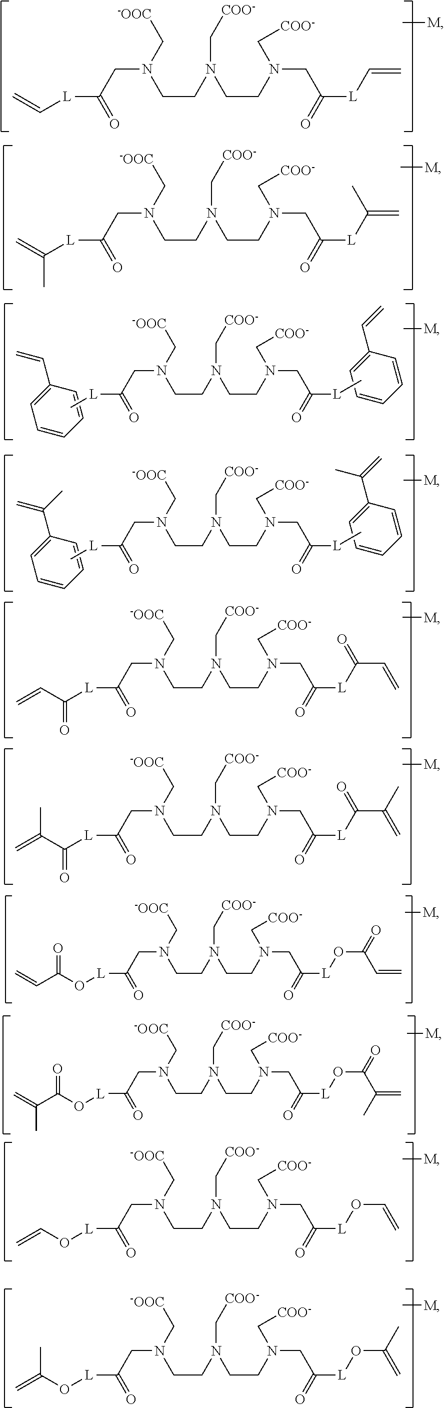

or mixtures thereof.

For example, the mixture can comprise one or more said metal chelating monomers with different metals.

In other embodiments, the chelator comprises porphyrin or phthalocyanine.

For example, the chelator can be substituted or unsubstituted porphyrin.

In some embodiments, the porphyrin and phthalocyanine are each independently substituted with C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, carboxylic acid, or mixtures thereof.

When the chelator comprises or is porphyrin or phthalocyanine, the metal can be a soft metal. For example, the soft metal can be cadmium, cobalt, copper, iron, zinc, nickel, tin, osmium, palladium, platinum, gold, thallium, mercury, or lead, including isotopes thereof, as well as mixtures thereof. In some embodiments, the soft metal has an atomic mass of 80 amu or above.

In some embodiments, the metal-chelating monomer prior to polymerization is selected from

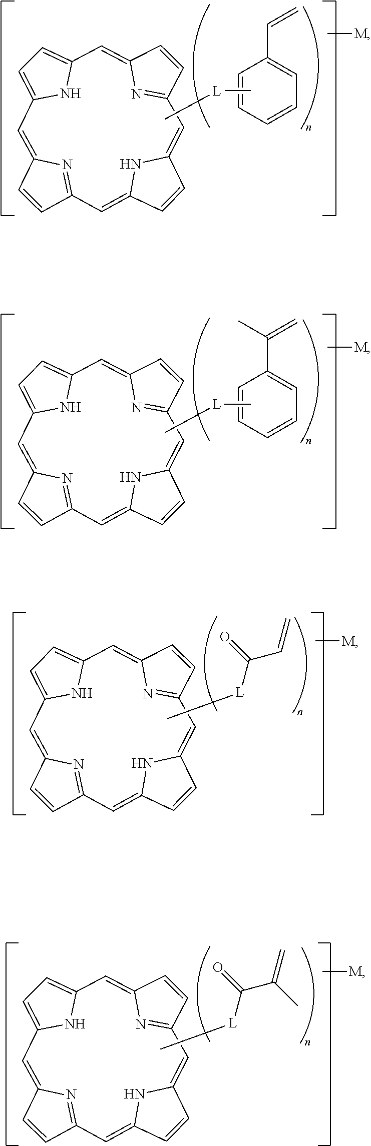

or mixtures thereof, and wherein n is an integer from 1 to 4.

For example, L can be aniline.

In some embodiments, n is 2 or at least 2.

In other embodiments, the metal-chelating monomer is selected from

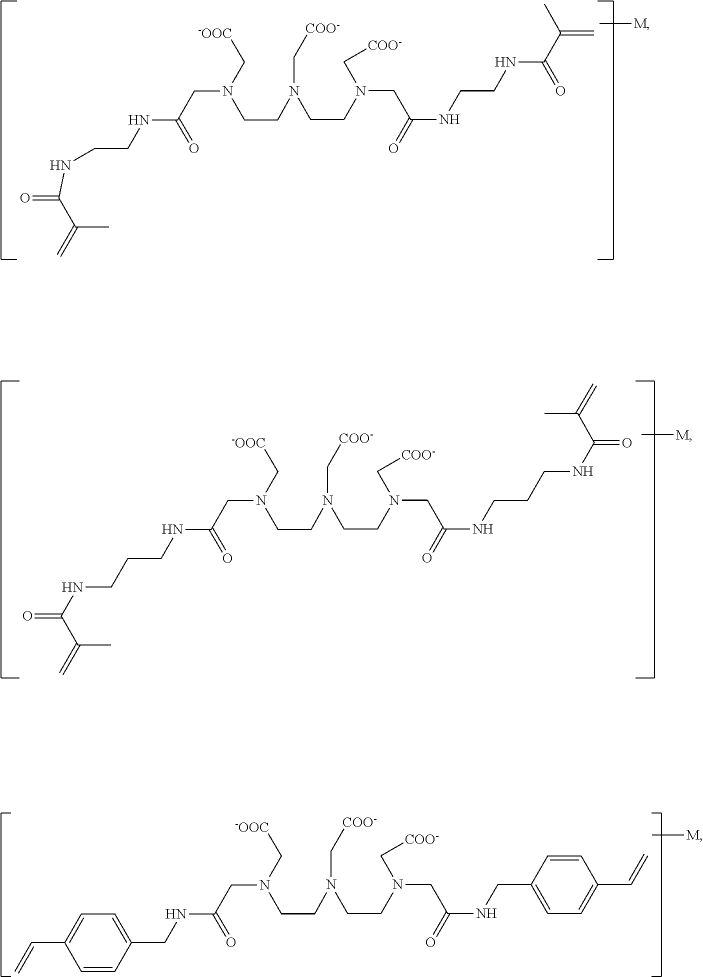

or mixtures thereof.

The microbead of the present disclosure can also further comprise a steric stabilizer. The steric stabilizer can be PVP, polyvinyl alcohol, hydroxymethylcellulose, hydroxyethylcellulose, hydroxypropylcellulose, polyacrylic acid, water soluble homopolymers of acrylic acid esters, water soluble homopolymers of methacrylic acid esters, water soluble homopolymers of acrylamide, homopolymers of methacrylamide, water soluble copolymer steric stabilizers, or mixtures thereof.

In some embodiments, the water-soluble copolymer steric stabilizer is selected from copolymers of acrylic acid ester, methacrylic acid ester, acrylamides or methacrylamide with methyl acrylate and/or ethyl acrylate, or mixtures thereof.

The copolymer can also be crosslinked, as is achieved for example when the metal-chelating monomer and/or the structural monomer have two or more polymerizable groups.

For example, a microbead of the present disclosure can comprise a plurality of metals. For example, each metal can be incorporated by the same type of a different type of metal-chelating monomer. The metal can be a plurality of metals.

In some embodiments, the plurality of metals comprises one or more enriched isotopes.

In some embodiments, the plurality of metals comprises at least 2 metals, at least 3 metals, or at least 4 metals.

In other embodiments, the amount of each metal of the plurality of metals is within about 20% or about 10% of the amount of another metal of the plurality of the metals. For example, this can be determined on a population of microbeads or a single microbead through mass cytometry.

The metal can, for example, be a transition metal (i.e., a metal from groups 3-12 of the periodic table, or from the lanthanide or actinide series). The metal can, for example, be indium, bismuth, or a rare earth metal. The rare earth metal can for example be a lanthanide metal, yttrium, or mixtures thereof. In some embodiments, the metal is indium, bismuth, a soft metal, or a rare earth metal. The soft metal can be cadmium, cobalt, copper, iron, zinc, nickel, tin, osmium, palladium, platinum, gold, thallium, mercury, lead, and isotopes thereof, as well as mixtures thereof.

In other embodiments, the metal comprises a rare earth metal that is selected from Y, La, Ce, Pr, Nd, Pm, Sm, Eu, Gd, Tb, Dy, Ho, Er, Tm, Yb, Lu, distinct isotopes thereof, and mixtures thereof.

In yet other embodiments, the metal is selected from La, Ce, Pr, Nd, Pm, Sm, Eu, Gd, Tb, Dy, Ho, Er, Tm, Yb, Lu, distinct isotopes thereof, and mixtures thereof.

In yet other embodiments, the rare earth metal is selected from 89Y, 139La, 136Ce, 138Ce, 140Ce, 142Ce, 141Pr, 142Nd, 143Nd, 145Nd, 146Nd, 148Nd, 145Pm, 144Sm, 149Sm, 150Sm, 152Sm, 154Sm, 151Eu, 153Eu, 154Gd, 155Gd, 156Gd, 157Gd, 158Gd, 160Gd, 152Gd, 159Tb, 156Dy, 158Dy, 160Dy, 161Dy, 162Dy, 163Dy, 164Dy, 165Ho, 162Er, 164Er, 166Er, 167Er, 168Er, 170Er, 169Tm, 168Yb, 170Yb, 171Yb, 172Yb, 173Yb, 174Yb, 176Yb, 175Lu, or mixtures thereof.

The metal can be distributed throughout the microbead substantially uniformly. It can also be compartmentalized, for example in the interior of the microbead.

In some embodiments, the microbead has a glass transition temperature (as measured, for example, by differential scanning calorimetry (DSC)) of about 60° C. or above 60° C., optionally about 70° C. or above 70° C., about 80° C. or above 80° C., about 90° C. or above 90° C., about 100° C. or above 100° C., about 115° C. or above 115° C., about 125° C. or above 125° C., or about 135° C. or above 135° C. For example, the DSC result is obtained on the second or third heating scan at a scan rate of about 10° C./min to about 20° C./min.

In other embodiments, the microbead has a diameter of about 0.6 μm to about 20 μm, about 1 μm to about 15 μm, about 2 μm to about 10 μm, or about 2 μm to about 6 μm. In some embodiments, the microbeads of the present disclosure have a size suitable for mass cytometry.

In yet other embodiments, the microbead is colloidally stable in water. For example, the microbeads of the present application are substantially stable upon storage in buffers and/or in physiological media. For example, substantially stable in buffers and/or physiological media means there is no significant leakage or less than 1% leakage of metals upon storage in buffers and/or physiological media.

In some embodiments, a surface of the microbead comprises functionalization for attachment to a biomolecule. The functionalization can be introduced by adding a coating layer.

In some embodiments, the attachment is covalent attachment. For example, as shown in the Examples, the microbeads can be coated with silica, and functionalized with reactive functional groups, such as a carboxylic acid group. A biomolecule can be added to the microbead.

A biomolecule may be classified as a protein, an oligonucleotide, a lipid, a carbohydrate, or a small molecule. Alternatively or in addition, a biomolecule may be classified by its functionality. The biomolecule is not particularly limited and different functionalizations can be used to conjugate the biomolecule to the microbead. For example, an oligonucleotide may be a single stranded DNA molecule, optionally cDNA that hybridizes under stringent conditions to a target nucleic acid analyte (e.g. a sample nucleic acid biomolecule) or the oligonucleotide can be an aptamer. For example, a biomolecule may be an oligonucleotide that specifically hybridizes a target oligonucleotide, such as a target mRNA endogenous to a sample (e.g. hybridizes to the sample oligonucleotide). Hybridization may be of a sequence that is more than 8, more than 10, more than 15, or more than 20 nucleotides.

In certain aspects, a biomolecule may be classified by its functionality. For example, a biomolecule may be an affinity reagent, an antigen (e.g., an analyte specifically bound by an affinity reagent), or an enzyme substrate. An affinity reagent may be an antibody (e.g., or fragment thereof), aptamer, receptor (e.g., or portion thereof), or any other biomolecule that specifically binds a target (e.g., an avidin, such as streptavidin, that specifically binds biotin). For example, a bead may be associated with an antibody may be used to detect the presence of its target antigen in a sample, such as the presence of a cytokine, viral protein, cancer biomarker, or the like. In certain methods and kits, a microbead may be functionalized with an avidin for attachment of another biomolecule functionalized with biotin (e.g., to allow a bead to be adapted to any of a number of different assays). An antigen may be a protein (or peptide sequence thereof) comprising an epitope that is specifically bound by an affinity reagent such as an antibody. For example, a bead may be attached to a viral antigen (such as a viral protein sequence), and may be used to detect the presence of antibodies in the sample that specifically bind the viral antigen, as described further herein. An enzyme substrate may be any substrate that is acted on by a specific enzyme, such as by an oxidoreductase, transferase, hydrolase, lyase, isomerase or ligase. For example, a substrate may be a protein (e.g., or a peptide sequence thereof) that is a substrate for an enzyme such as a protease, phosphatase, kinase, methyltransferase, demethylases. Non-protein substrates include, for example, a double stranded oligonucleotide comprising a restriction sequence cleavable by a restriction enzyme or a site (such as a nick) for DNA repair, an oligonucleotide sequence comprising a sequence targeted by a DNA methyltransferase, or any non-protein substrate known to one of skill in the art. For example, a bead may be attached to a substrate and exposed to a sample comprising an enzyme that modifies the substrate, and modification (or lack thereof) of the substrate may be detected (e.g., as described further herein).

In some embodiments, the affinity reagent is or comprises an antibody or a binding fragment thereof. The antibody can for example be a biotinylated antibody or binding fragment and can be added directly or indirectly to the microbead.

For example, as demonstrated in the Examples, Neutravidin (NAv) was covalently conjugated to the COOH groups on the silica coated microbead surface by EDC/NHS coupling. A biotinylated antibody was attached to the NAv-modified microbead surface through the strong biotin-avidin affinity.

Accordingly, the affinity reagent can be avidin or related biotin binding molecules such as streptavidin, NeutrAvidin and CaptAvidin. Microbeads comprising such an affinity reagent can be customized using a biotinylated antibody specific for a target analyte of interest.

In some embodiments, the affinity reagent optionally the antibody, is specific for a cytokine, optionally a chemokine, an interferon, a lymphokine, a monokine, an interleukin, such as IL-1-36, tumor necrosis factor and colony stimulating factors.

The antibody may also be specific for a pathogenic protein such as a viral, bacterial or fungal pathogen. Such microbeads can be used to detect for the presence of such pathogens or their products in samples such as environmental or patient samples.

In other embodiments, the antigen is a viral antigen. Such microbeads comprising a viral antigen can be used for example to detect the presence of viral antibodies in patient samples. Similarly, antigens such as those from other pathogens.

In yet other embodiments, the copolymer of the microbead further comprises a third monomer, the third monomer being present at least on the surface of the microbead and comprising at least one reactive functional group. In some embodiments, the microbead is functionalized through the at least one reactive functional group comprised in the third monomer, for example through 3-stage dispersion polymerization. In some embodiments, the third monomer is substituted or unsubstituted acrylic acid or methacrylic acid. An example of 3-stage dispersion polymerization is described in Abdelrahman et al., JACS, 2009, 15276, the content of which is hereby incorporated by reference.

For example, the surface of the microbead can be functionalized with a reactive functional group.

In some embodiments, the reactive functional group is on the coating of silicon dioxide.

In other embodiments, the reactive functional group is selected from amine, thiol, alcohol, aldehyde, carboxylic acid, epoxide, vinyl, alkyne, maleimide, or a click chemistry moiety (e.g. such as dibenzocyclooctyne (DBCO), azide, trans-cyclooctenes (TCO), or tetrazine, or derivatives thereof) or mixtures thereof.

In yet other embodiments, the functionalization comprises a coating of silicon dioxide on the surface of the microbead, optionally the functionalization further comprises functionalizing the coating of silicon dioxide.

In some embodiments, the surface of the microbead is functionalized with a reactive functional group.

In other embodiments, the reactive functional group is on the coating of silicon dioxide.

For example, the reactive functional group can be selected from amine, thiol, alcohol, aldehyde, carboxylic acid, epoxide, vinyl, alkyne, maleimide, or mixtures thereof.

It can be appreciated that functionalization of the silicon dioxide coating can be done using methods known in the art. For example, a method of functionalization of microbead by silicon dioxide coating is described in US 2019/0091647, the content of which is hereby incorporated by reference.

In some embodiments, the attachment to the biomolecule is non-covalent attachment.

In other embodiments, the surface of the microbead is functionalized with avidin, streptavidin, neutravidin, or mixtures thereof.

In yet other embodiments, the surface of the microbead is conjugated to the biomolecule.

In some embodiments, the metal provides a barcode that identifies the biomolecule.

In some embodiments, an interior structure of the microbead comprises the copolymer.

In other embodiments, the metal-chelating monomer chelates a single metal atom and not a plurality of metal atoms.

In some embodiments, the microbead comprises a polymer seed that does not comprise the metal-chelating monomer. In some embodiments, the polymer seed has an interior space formed by swelling the polymer seed, and the copolymer is present at least within the interior space of the polymer seed.

In some embodiments, the polymer seed comprises a structural monomer; optionally wherein the structural monomer of the polymer seed is identical in structure to the structural monomer of the copolymer.

In another aspect, the present disclosure includes a population of microbeads of the present disclosure.

In some embodiments, the population has a size distribution having a coefficient of variation (CV) of about 10% or less than 10%.

In other embodiments, the coefficient of variation is of less than 5%.

In some embodiments, each microbead comprises a plurality of metals, the average amount across the population of microbeads of each metal of the plurality of metals is about 10% or within 10% of the average amount of another metal of the plurality of metals.

In other embodiments, the plurality of metals comprises one or more enriched isotopes.

In some embodiments, the amount of each metal of one microbead of the population of microbeads is about 20% or within 20%, or about 10% or within 10%, or about 5% or within 5% of the amount of the same metal of another microbead of the population of microbeads.

In other embodiments, the amount of each metal of the population of microbeads has a distribution of a coefficient of variation of about 20% or less than 20%.

In yet other embodiments, the amount of each metal of the population of microbeads has a distribution of a coefficient of variation of about 10% or less than 10%.

In some embodiments, the microbeads of the population of microbeads comprise the same metal in substantially the same amount.

In some embodiments, the same metal is a plurality of metals and the microbeads comprise each metal of the plurality of metals in substantially the same amount.

A further aspect is a composition comprising the microbead or plurality of microbeads. The composition can for example be a buffered solution, for example buffered to a pH of about 7.

The buffered solution can comprise ammonium acetate and like buffers. The composition may also comprise PVP.

In an embodiment, the composition is an aqueous colloidal suspension.

The composition can comprise one or more components selected from stabilizers, preservatives, buffers, and mixtures thereof.

In another aspect, the present disclosure includes a kit comprising the microbead, the plurality of distinct populations of microbeads and/or compositions of the present disclosure.

In some embodiments, each population of microbeads is distinguishable from another population of microbeads based on the metal or the plurality of metals of the microbeads.

In some embodiments, the microbeads of at least one population (e.g., each population) of microbeads comprise a metal or a plurality of metals different from the metal or the plurality of metals of the microbeads of another population of microbeads.

In some embodiments, the microbeads of at least one population (e.g., each population) of microbeads comprise a plurality of metals at a ratio different from the microbeads of another population of microbeads.

In some embodiments, the microbeads of each population of microbeads are conjugated to a different biomolecule. For example, the microbead can be used as a barcoding agent for the biomolecule attached to the microbead through the nature of the metal or plurality of metals in the microbead.

In another aspect, the present disclosure includes a microbead prepared by a method of the present disclosure.

In some embodiments, the structural monomer and metal chelating monomer may have been copolymerized according to any of the methods described herein. Of note, the structural monomers and/or metal chelating monomers used in the methods described herein may have any of the aspects described in this section.

A kit of the present disclosure may include any of the microbeads, or populations of microbeads, described herein. Microbeads, or kits thereof, can also include additional aspects or for mass spectrometry assays as described further herein. Aspects of the present disclosure also include additional methods, such as mass spectrometry assays, as described further herein.

### III. Methods of the Present Disclosure

In another aspect, the present disclosure includes a method of preparing a metal-encoded microbead comprising
polymerizing a structural monomer in the presence of a steric stabilizer in a nucleation stage to obtain a first mixture comprising polymerized structural monomer, unpolymerized structural monomer, and the steric stabilizer;
combining the first mixture with a metal-chelating monomer comprising a metal and a chelator attached to at least one polymerizable end group to obtain a second mixture,
wherein the chelator coordinates the metal at least at 3 sites and wherein the metal-chelating monomer is polymerizable with the structural monomer; and
polymerizing the second mixture to form a copolymer of the microbead;
wherein the structural monomer does not comprise the chelator.

It can be appreciated that for more efficient polymerization, the structural monomer and the metal-chelating monomer should be soluble in the reaction medium. Further, it can be appreciated that when a monomer is substituted for example with a substituent that may interfere with polymerization (e.g. halo, amine, alcohol), the substituent can be temporarily protected prior to and/or during polymerization using protective groups known in the art. Subsequent to polymerization, the protective groups can be selective removed and the substituents selectively deprotected using methods known in the art.

In some embodiments, the metal is a plurality of metals.

In other embodiments, the structural monomer is polymerized in the nucleation stage to about 5% to about 20% completion based on the structural monomer.

In yet other embodiments, the polymerizing of the second mixture occurs to about 75% to about 100% completion, about 80% to about 99% completion, about 85% to about 95% completion, about 85% to about 93% completion based on the structural monomer.

In some embodiments, the structural monomer is as defined herein.

In some embodiments, the metal-chelating monomer is as defined herein.

In some embodiments, the steric stabilizer is as defined herein.

In some embodiments, the metal is as defined herein.

In some embodiments, the method further comprises functionalizing the microbead.

In some embodiments, the functionalizing of the microbead comprises
mixing the polymerized second mixture with a third monomer to obtain a third mixture, the third monomer comprising a reactive functional group; and
polymerizing the third mixture.

For example, the reactive functional group can be selected from amine, thiol, alcohol, aldehyde, carboxylic acid, epoxide, vinyl, alkyne, maleimide, or mixtures thereof. It can be appreciated that certain reactive functional groups may interfere with the polymerization process and can be protected prior and/or during polymerization using protective groups known in the art. For example, amines and thiols can be protected by protective groups. For example, monomers substituted with reaction functional groups can be used in a protected form such that the reactive functional groups would not interfere with the polymerization process. Optionally, protected reactive functional groups can be deprotected using methods known in the art.

In some embodiments, the third monomer is selected from optionally substituted acrylic acid, optionally substituted methacrylic acid, and mixtures thereof.

In other embodiments, the functionalizing of the microbead comprises coating the microbead with silicon dioxide.

In other embodiments, the functionalizing of the microbead further comprises

functionalizing the coating of silicon dioxide.

For example, the functionalization of the coating of silicone dioxide can comprise reacting the coating of silicon dioxide with an organic silane. For example, the organic silane can be selected from chlorosilane, alkoxysilane, derivatives thereof, and mixtures thereof.

In some embodiments, the reacting the coating of silicone dioxide with the organic silane is done in presence of a catalyst. For example, the catalyst can be selected from ammonia, hydroxide, organic amine, or mixtures thereof.

In some embodiments, the organic silane comprises a reactive functional group.

In some embodiments, the reactive functional group is selected from amine, thiol, alcohol, aldehyde, carboxylic acid, epoxide, vinyl, alkyne, maleimide or mixtures thereof.

In some embodiments, the organic silane is APTES.

In other embodiments, the method further comprises conjugating the microbead to a biomolecule.

In some embodiments, the biomolecule is as defined herein.

In some embodiments, the microbead has a diameter of about 0.6 μm to about 20 μm, about 1 μm to about 15 μm, about 2 μm to about 10 μm, about 2 μm to about 6 μm.

In some embodiments, the microbead is a microbead of the present disclosure.

In some embodiments, an interior structure of the microbead comprises the copolymer.

In another aspect, the present disclosure includes a method of preparing metal-encoded microbeads, the method comprising:
providing an aqueous dispersion of swellable seed particles and an anionic surfactant;
   contacting the aqueous dispersion with monomers comprising a structural monomer and a metal-chelating monomer, wherein the metal-chelating monomer comprises a metal and a chelator attached to at least one polymerizable end, and wherein the chelator coordinates the metal at least at 3 sites; and
allowing the monomers to diffuse into the seed particles to form an aqueous dispersion of swollen seed particles; and
initiating polymerization of the monomers in the aqueous dispersion of swollen seed particles;
wherein the structural monomer does not comprise the chelator.

In another aspect, the present disclosure includes a method of preparing a metal-encoded microbead comprising
providing an aqueous dispersion comprising a swellable polymer seed, an organic compound, an anionic surfactant, and optionally an organic solvent in which the organic compound is soluble;
allowing the organic compound to diffuse into the swellable polymer seed;
contacting the aqueous dispersion with a mixture comprising a structural monomer and a metal-chelating monomer; optionally the mixture further comprises a steric stabilizer and/or a polymerization initiator; and
polymerizing the mixture to obtain a copolymer of the microbead;
wherein the organic compound has a molecular weight below 5000 Da and a water solubility at 25° C. of less than 10-2 g/L.

In another aspect, the present disclosure includes method of preparing a metal-encoded microbead comprising
preparing a swellable polymer seed by emulsion polymerization, wherein an anionic surfactant is used as emulsifier under substantially oxygen-free conditions;
contacting the swellable polymer seed with an aqueous dispersion comprising an organic compound, the anionic surfactant, and optionally an organic solvent in which the organic compound is soluble;
allowing the organic compound to diffuse into the swellable polymer seed;
contacting the aqueous dispersion with a mixture comprising a structural monomer and a metal-chelating monomer; optionally the mixture further comprises a steric stabilizer and/or a polymerization initiator; and
polymerizing the mixture to obtain a copolymer of the microbead;
wherein the organic compound has a molecular weight below 5000 Da and a water solubility at 25° C. of less than 10-2 g/L.

In some embodiments, the structural monomer is selected from the group consisting of acrylic monomers, methacrylate monomers and vinyl monomers selected from the group consisting of styrene, divinylbenzene (DVB), ethyl vinyl benzene, vinyl pyridine, amino-styrene, methyl-styrene, dimethylstyrene, ethyl styrene, ethyl-methyl-styrene, p-chlorostyrene and 2,4-dichlorostyrene.

In some embodiments, the aqueous dispersion of swollen seed particles further comprises a steric stabilizer.

In some embodiments, the steric stabilizer is polyvinylpyrrolidone.

In some embodiments, the providing the aqueous dispersion of swellable seed particles comprises preparing monodisperse swellable seed particles by emulsion polymerization.

In some embodiments, the aqueous dispersion of swellable seed particles further comprises an organic compound with a molecular weight below 5000 Dalton and a water solubility at 25° C. of less than 10-2 g/L; and optionally an organic solvent in which said organic compound is soluble.

In some embodiments, the swellable seed particles are monodisperse swellable seed oligomer particles.

For example, the anionic surfactant can be an alkyl sulfate or an alkyl sulfonate. In some embodiments, the anionic surfactant is a C8-16alkyl sulfate or sulfonate or salts thereof. For example, the anionic surfactant can be decylsulfate, dodecylsulfate, decylsulfonate, dodecylsulfonate or salt thereof. In some embodiments, the anionic surfactant is sodium dodecyl sulfate or sodium decylsulfate.

In some embodiments, the structural monomer is as defined herein.

In some embodiments, the metal-chelating monomer is as defined herein.

In some embodiments, the organic compound is a polymerization initiator.

In some embodiments, the polymerization initiator is a peroxide, an azo compound, or mixtures thereof.

In some embodiments, the organic solvent is a non-polymerizable solvent selected from alcohol, ether, ketone, dialkylsulfoxides (e.g. DMSO), dialkylformamides (e.g. DMF), or mixtures thereof.

### IV. Synthesis of the Monomer of the Present Disclosure

The monomers of the present disclosure can be prepared by various synthetic processes. The choice of particular structural features and/or substituents may influence the selection of one process over another. The selection of a particular process to prepare a given monomer is within the purview of the person of skill in the art. Some starting materials for preparing compounds described in the present disclosure are available from commercial chemical sources. Other starting materials, for example as described below, are readily prepared from available precursors using straightforward transformations that are well known in the art. In the Schemes below showing the preparation of the second monomer of the application, all variables are as defined in the present description, unless otherwise stated.

The compounds of Formula (I) can be prepared for example according to the processes illustrated in the Schemes below. In the structural formulae shown below, the variables are as defined in Formula (I) unless otherwise stated. A person skilled in the art would appreciate that many of the reactions depicted in the Schemes below would be sensitive to oxygen and/or water and would know to perform the reaction under an anhydrous, inert atmosphere if needed. Reaction temperatures and times are presented for illustrative purposes only and may be varied to optimize yield as would be understood by a person skilled in the art.

Accordingly, in an embodiment, the compounds of Formula I are prepared as shown in Scheme A. The chelator or the ligand of Formula A is attached to one or more linkers with a polymerizable end group of Formula B to form a monomer capable of metal chelation of Formula C. Then, a metal D can be chelated to the monomer of Formula C to form the metal-chelating monomer of Formula I.

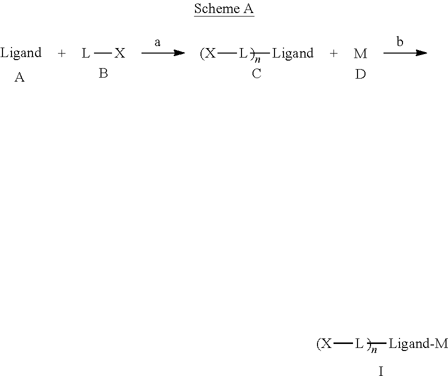

In some embodiments, the ligand of Formula A can comprise one or more carboxylic acid groups. Accordingly, step a in Scheme A can be accomplished according to Scheme B. The ligand of Formula E can be attached to one or more linkers with a polymerizable group of Formula B through an amide bond formation or an esterification to obtain the monomer capable of metal chelation of Formula F. It can be appreciated that amide bond formation can be carried out using methods known in the art for example through the formation of an activated ester.

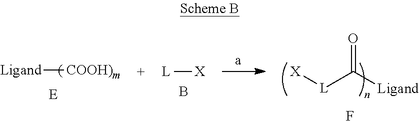

It can be appreciated that the metal chelation step b can be carried our using methods known in the art. For example, in some embodiments, the monomer capable of metal chelation of Formula C or F and the metal of Formula I can each be dissolved into a suitable solvent. Suitable solvent can be selected by a person skilled in the art and can include water. Metal of Formula D can be salts of the metal, for example halide salts of the metal. Solutions of the monomer capable of metal chelation and of the metal can be mixed together. For example, the pH of the resulting mixture can be monitored and adjusted to a suitable pH. In some embodiments, a suitable pH includes a pH of about 5.5 to about 7.5, or about 6. The resulting mixture can be stirred until the metal-chelating monomer of Formula I forms.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### V. Mass Spectrometry Methods and Kits

The microbeads and kits described above may comprise additional aspects for use in mass spectrometry assays. Described below are methods and kits for mass spectrometry assays, which may include or use any suitable microbeads or kits described elsewhere herein. Suitable assays include assays to detect a target or sample biomolecule, such as target oligonucleotides, proteins (e.g., cytokines, cancer biomarkers, or antibodies to a specific antigen), or enzymes (e.g., activity of enzymes such as kinases, phosphatases, proteases, or any other enzyme of interest that modifies a substrate biomolecule attached to a microbead). Assay may use hybridization and/or a sandwich ELISA format to detect a target sample biomolecule. Suitable assays also include analysis of cells (e.g., use of microbeads as a standard for calibration, normalization or quantitation in a mass cytometry assay).

Microbeads (and kits or methods thereof) of the present disclosure may be used for optical detection (e.g., fluorescence-based detection). Alternatively, microbeads (and kits or methods thereof) of the present disclosure may be analyzed by elemental analysis (e.g., mass spectrometry). As such, the microbeads may exhibit low fluorescence (e.g., may have a fluorescence quantum yield less than 0.2, less than 0.1, less than 0.05, or less than 0.02, across the visible and/or UV range) but would still be suitable for analysis by mass spectrometry (e.g., atomic mass spectrometry such as by ICP-MS) or another form of elemental analysis (e.g., by ICP-OES, by x-ray dispersion spectroscopy).

Assay methods and kits described herein may be for analysis of samples by mass spectrometry. Suitable samples include any biological sample, such as a cell sample (e.g., a suspension of cells or a tissue section) or a biological fluid (e.g., comprising sample biomolecules in suspension). A sample may be a cell line, cell culture supernatant, or may be harvested from an organism such as a human, rodent or other mammal. A sample may be a blood sample, such as whole blood, serum, plasma, or peripheral blood mononuclear cells (PBMCs). A sample may be a saliva sample, nasal swab, excision (e.g., biopsy of solid tissue). A sample may comprise whole cells or may be homogenized from (e.g., a lysate from) a sample containing whole cells. In certain aspects, a sample may be a purified sample biomolecule that is characterized by an assay, or using a kit, described herein. For example, an assay or kit may screen potential sample biomolecules that are drug candidates. Of note, additional steps may be performed in any method described herein, such to remove unbound sample biomolecules from microbeads, unbound reporters from microbeads, or unbound mass tagged antibodies from cells.

In imaging mass cytometry (IMC), a sample may be a tissue section that is labeled with mass tags (e.g., by mass tagged antibodies) for analysis by imaging mass spectrometry (e.g., LA-ICP-MS or SIMS). In suspension mass cytometry, particles in suspension (such as cells and/or beads) may be introduced to a mass cytometer (e.g., an ICP-MS system) for analysis.

Mass spectrometry of the subject application may be atomic mass spectrometry. A mass spectrometer of the subject application may simultaneously detect a plurality of mass channels. Different mass channels may correspond to a metal or isotope thereof from different mass tags. Such simultaneous mass spectrometers may be, for example, a time-of-flight mass spectrometer (TOF-MS) or a magnetic sector mass spectrometer. A mass spectrometer may atomize the sample for atomic mass spectrometry. For example, ICP, laser ablation ICP, secondary ion (e.g., for secondary ion mass spectrometry (SIMS)) or any suitable ionization source may be used to atomize a sample. A mass cytometer may specifically detect metals from mass tags and/or microbeads described herein, and may, for example, comprise ion optics for filtering out lighter atoms (e.g., endogenous elements such as C, N, O, and light metals, and optionally further plasma gas elements such as Argon and Argon dimer if the mass spectrometer is an ICP system). Exemplary mass cytometers are described further in US patent publication numbers 20050218319 and 20160049283, which are incorporated herein by reference.

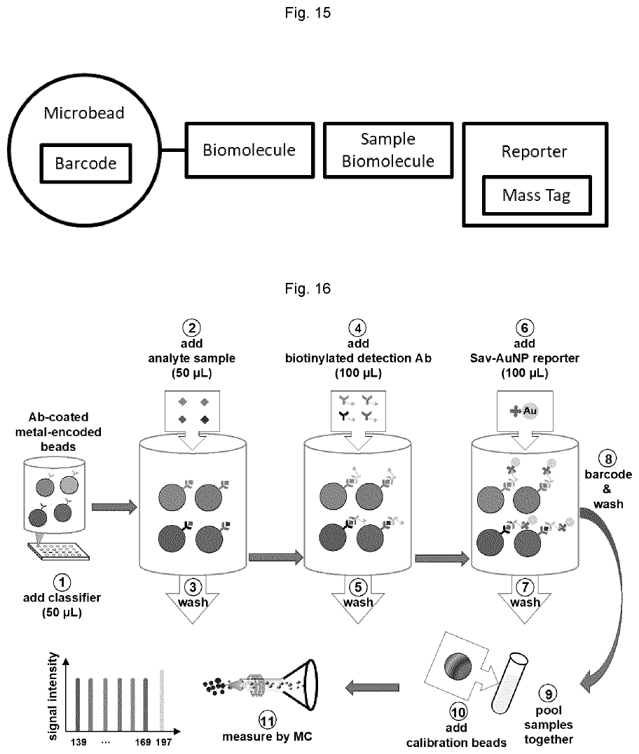

An exemplary assay scheme is shown in FIG. 15. A copolymer microbead of the present disclosure may comprise a barcode. The barcode may be a plurality of metals or enriched metal isotopes chelated by a metal chelating monomer. The barcode may be an assay barcode (i.e., that identifies a sample biomolecule the microbead is functionalized to bind to). Alternatively or in addition, the microbead may comprise a sample barcode used to identify a sample the microbead was, or will be, mixed with. The microbead may be attached to (e.g., covalently bound to) a biomolecule (e.g., a capture biomolecule) that specifically binds to sample biomolecule. The biomolecule may include, for example, an affinity reagent such as an antibody that specifically binds a sample antigen biomolecule of interest such as a viral particle, cytokine, cancer biomarker. The biomolecule may include, for example, an oligonucleotide such as a ssDNA oligonucleotide that has a sequence that specifically hybridizes to a sample oligonucleotide biomolecule of interest. Mixing a plurality of such beads with a sample may allow the biomolecules of each bead to bind to their respective sample biomolecules, as shown in FIG. 15. Further, as shown in FIG. 15, a reporter may be bound to the sample biomolecule (e.g., before, after or during the step of binding the sample biomolecule to the biomolecule attached to the bead). The reporter may include a mass tag. As such, the methods of the present disclosure may include binding a sample biomolecule to a biomolecule (e.g. capture biomolecule) attached to a barcoded microbead, and may further include binding a mass tagged reporter to the sample biomolecule. The barcode and mass tag may then be detected by atomic mass cytometry as described herein. Likewise, certain kits described herein may include barcoded microbeads optionally attached to a biomolecule that specifically binds a sample biomolecule, and the kit may further include a reporter that associates a mass tag with the sample biomolecule.

In some embodiments, a kit includes a population of microbeads, or a plurality of distinct populations of microbeads, as described in any of the other embodiments herein. In certain aspects, each population of microbeads may be distinguishable (e.g., by atomic mass spectrometry) from another population of microbeads based on the metal or the plurality of metals of the microbeads. For example, the microbeads of at least one population (e.g., each population) of microbeads comprise a metal or a plurality of metals different from the metal or the plurality of metals of the microbeads of another population of microbeads, or may comprise a plurality of metals at a ratio different from the microbeads of another population of microbeads.

In some embodiments, the microbeads of each population of microbeads in a kit are conjugated to a different biomolecule. In some embodiments, a kit may further include a reporter comprising a mass tag, such as a reporter that is capable of specifically binding a sample biomolecule specifically bound by at least one of the different biomolecules. Different biomolecules attached to microbeads of the kit may specifically bind to different sample biomolecules. Sample biomolecules may be any biomolecule described herein that is present in a sample. Such sample biomolecules may be a protein (e.g., or peptide thereof), oligonucleotide, lipid, carbohydrate or small molecule. A protein may be, for example, an antibody or a cytokine. An oligonucleotide may be a genomic DNA sequence, a cDNA sequence or an RNA sequence, such as an mRNA sequence. The oligonucleotide may also be a DNA RNA hybrid and/or comprise one or more modified residues.

A sample biomolecule may include an oligonucleotide (e.g., an RNA), and at least one of the different biomolecules of the microbeads may be an oligonucleotide (e.g., a ssDNA) that specifically hybridizes to the sample biomolecule (e.g. target analyte). For example, the reporter comprises a plurality of oligonucleotides that hybridize to indirectly bind a plurality of mass tagged oligonucleotides to the sample biomolecule. Alternatively, a reporter comprising a mass tag may directly hybridize to the sample biomolecule. In certain aspects, a sample biomolecule may be a biomolecule other than an oligonucleotide, such as an antigen (e.g., a protein such as a cytokine), and the reporter may comprise an oligonucleotide conjugated to an antibody, where the antibody is able to bind the antigen and the oligonucleotide conjugated to the antibody is bound, directly or indirectly (e.g., through hybridization) to mass tag oligonucleotide of the reporter. Such indirect hybridization may allow for association of multiple mass tags with a sample biomolecule, such as through hairpin chain reaction, branched in-situ hybridization, or any other suitable method. As such, in some embodiments, a reporter as described herein may (or may not) include a system of separate biomolecules that together associate a mass tag with a sample biomolecule.

In some embodiments, at least one of the different biomolecules attached to a microbead is an antibody (e.g., or a fragment thereof, such as a nanobody). For example, the sample biomolecule is a viral particle, wherein the at least one of the different biomolecules is a first antibody that specifically binds the viral particle, and wherein the reporter comprises a second antibody that specifically binds the viral particle.

In some embodiments, at least one sample biomolecule is a cytokine, for example wherein at least one of the different biomolecules attached to a microbead is a first antibody that specifically binds the cytokine, and wherein the reporter comprises a second antibody that specifically binds the cytokine.

In some embodiments, the cytokines are selected from IL-18, IL-1F4, TNFα, IL-6, IFNγ, IL-4, CD163, CXCL-9/MIG, IL-10, IL-1β, and combinations thereof.

In some embodiments, at least one sample biomolecule is a cancer biomarker (for example a prostate specific antigen), wherein the at least one of the different biomolecules is a first antibody that specifically binds the cancer biomarker, and wherein the reporter comprises a second antibody that specifically binds the cancer biomarker.

In some embodiments, the at least one of the different biomolecules includes a viral antigen, wherein the sample biomolecule is an antibody that specifically binds the viral antigen, and wherein the reporter comprises a secondary antibody that binds to the sample biomolecule.

The methods and kits of the present disclosure may include a plurality of different reporters, wherein each of the different reporters is capable of binding a sample biomolecule specifically bound by a different biomolecule. A plurality of different reporters may each comprise the same mass tag (detected in the same mass channel), or a different mass tag (detected in different mass channels). The mass tag may include a metal nanoparticle, such as a metal nanocrystal (e.g., a nanogold particle), a quantum dot, a polymer nanoparticle, or the like. A nanoparticle may be at or less than 100 nm, less than 50 nm, less than 20 nm, less than 10 nm in diameter, such as between 2 nm and 100 nm or between 5 and 50 nm. The mass tag may include a metal chelating polymer, such as a linear or branched polymer comprising metal binding (e.g., metal chelating) pendant groups. Other mass tags may be suitable, such as an organotellurium polymer mass tag in which a tellurium atom is covalently bound to carbon atoms of the polymer, are also within the scope of the present disclosure. A mass tag may have one or more atoms of a metal element or enriched isotope thereof. Mass tags may be conjugated to a biomolecule (e.g., a biomolecule of a reporter that binds to a sample biomolecule) through any suitable conjugation means described herein or known to one of skill in the art.

In some embodiments, a biomolecule attached to a microbead is an enzyme substrate for a sample biomolecule. A method or kit of the present disclosure may further include a reporter that specifically binds (e.g. is capable of specifically binding) to the enzyme substrate when it has been modified by the sample biomolecule or that specifically binds (e.g. is capable of specifically binding) to the enzyme substrate when it has not been modified by the sample biomolecule. For example, the enzyme substrate may include a kinase substrate, the sample biomolecule (e.g. target analyte) may be a kinase that phosphorylates the kinase substrate, and the reporter may be a phosphorylation specific antibody that binds the phosphorylated substrate. In another example, the enzyme substrate includes a mass tag that is removed when the enzyme substrate is modified by the sample biomolecule, such as a peptide sequence that is cleaved by a sample biomolecule that is a protease. In some embodiments therefore, the absence of a signal of a mass tag may indicate the presence of a sample biomolecule enzyme. Examples of enzyme assays are described in US patent publication number 20070190588, which is incorporated herein by reference.

In some aspects of the kits and methods of the present disclosure, microbeads from a plurality of different populations are in admixture (in a first mixture of microbeads). Aspects may further include a second mixture of microbeads comprising the same biomolecules as the first mixture of microbeads, wherein the microbeads of the first mixture comprise a sample barcode that is different from the microbeads of the second mixture. The sample barcode is for example on the interior of the microbeads (e.g., may be a subset of metals chelated by the metal chelating monomer of the microbeads of the first and second mixture). Alternatively, therein the sample barcode may be on the surface of the microbeads of the first and second mixture. Methods or kits may include a plurality of sample barcodes in separate partitions that are functionalized to bind to the surface of microbeads from a plurality of different populations (e.g., where the sample barcodes of each partition are applied to the microbeads of a different mixture). Sample barcoded microbeads may be mixed together (e.g., after mixing with their respective samples but optionally before any mixture with a reporter), then analyzed by mass spectrometry. The mass spectrum from the sample barcode of each microbead may thereby be used to identify the sample it came from.

In certain aspects, kits or methods may further include a panel of mass-tagged antibodies in admixture with one another, wherein at least some antibodies of the panel are specific for cell surface markers (e.g., are used to bind cell surface proteins in a cellular sample). The plurality of antibodies may in admixture with microbeads of the kit, e.g., in a buffered solution or in a lyophilized form (e.g., less than 5% or less than 1% moisture by volume). A plurality of (e.g., each of) the mass-tagged antibodies comprise metals identical to metals chelated by metal chelating monomers of microbeads of the kit. However, the cells and microbeads described herein may be distinguishable by atomic mass spectrometry. For example, cells as described herein may include one or more metals (or enriched isotopes thereof) that no naturally occurring cells usually comprise, such as an iridium intercalator that binds to DNA of the cells. Microbeads may be analyzed alongside cells as a mass standard and/or to detect biomolecules (e.g., cytokines, antibodies, cancer biomarkers) in a sample solution (e.g., a cell culture supernatant or a serum).

In some embodiments a kit comprising one or more populations of microbeads as described herein further includes a steric stabilizer (e.g., in admixture with microbeads of the kit). As described herein, the steric stabilizer may be polyvinylpyrrolidone (PVP), such as at more than 0.05%, 0.1%, 0.2%, 0.5%, or 1% by weight, such as at or between about 0.05% and about 10%, about 0.1% and about 5%, about 0.2% and about 2% by weight. Alternatively, or in addition, microbeads of the kit may be in a solution buffered at a pH at or between about 4 and about 10, a pH at about or between about 5 and about 9, a pH at about or between about 6 and about 8, a pH greater than about 3, a pH greater than about 4, or a pH less than about 10.

In some embodiments, the microbeads of a kit of the present disclosure are lyophilized. For example, the microbeads of the kit are at less than 10%, less than 5%, less than 2%, less than 1%, less than 0.5%, less than 0.2%, less than 0.1%, or between about 0.05% and about 5% moisture by weight.

In some embodiments, microbeads may be fused to a solid support, such as for calibration (e.g. as reference particles), normalization or quantitation in imaging mass spectrometry (or imaging mass cytometry) as described further herein. The solid support may be any suitable support such a slide (e.g., a microscope slide, such as a transparent glass or quartz slide) or an adhesive film (e.g., for application to a microscope slide in any of the subject methods). The solid support may further comprise a biological sample.

In some embodiments, the solid support comprises at least 2, such as at least 3, at least 5, at least 10, at least 50, at least 100, at least 500, at least 1000, at least 2000, at least 5000, such as at least 10000 fused microbeads. The microbeads (reference particles) may all be the same, or the microbeads may differ in the metals or amounts thereof. When different microbeads are used, there will typically be multiple of each population of microbead.

The microbeads may be dispersed on the solid support such that substantially all of the microbeads are located individually (i.e. discretely) on the solid support, such that each fused microbead can be individually identified and sampled. Those skilled in the art will appreciate that the solid support may further comprise some fused microbeads that have agglomerated on the sample carrier and thus these agglomerates may be unsuitable for sampling for calibration and normalization of signal intensity. For example, up to about 2%, such as up to about 5%, up to about 8%, up to about 10%, up to about 15%, or up to about 20% of the fused microbeads may be agglomerated on the solid support. In other words, at least about 80% of the microbeads may be individually isolated, such as at least about 85%, at least about 90%, at least about 92%, or at least about 95%. Optical interrogation may identify which locations of the solid support have discrete microbeads, and can guide the acquisition by imaging mass spectrometer.

The step of fusing the at least one microbead to the solid support may comprise heating the solid support. In some embodiments, the step of fusing the at least one microbead with the sample carrier or solid support comprises heating the sample carrier or solid support at a temperature above the glass transition temperature of the microbead and subsequently cooling the sample carrier or solid support below the glass transition temperature of the microbead. In other words, the fusing of the at least one microbead to the sample carrier or solid support can occur by vitrification. In some embodiments, the sample carrier or solid support is heated to a maximum of up to 300° C., for example up to 275° C., up to 250° C., up to 225° C. or up to 200° C.

A kit of any embodiments described herein may further include one or more of a buffer (e.g., PBS, red blood cell lysis buffer, wash buffer, staining buffers, and/or buffers for reconstituting lyophilized reagents such as lyophilized microbeads, lyophilized reporters, lyophilized antibodies), anticoagulant (e.g., for processing a blood sample), fixation reagents, permeabilization reagents, or any reagents for performing the methods or examples described herein.

Method of the present disclosure include analyzing any microbeads described herein by mass spectrometry. For example, the microbeads (e.g., one or more populations of microbeads) may be an element standard used for calibrating a mass spectrometer and/or to normalization (e.g., normalization of a mass spectrum obtained from mass tags) or quantitation (e.g., quantitation of the number of antibodies in a cell or pixel) as described further herein.

In some embodiments, a method of mass spectrometry analysis includes: mixing the distinct populations of microbeads of the present disclosure with a sample, wherein microbeads of each population of microbeads are bound to a different biomolecule and wherein each population of microbeads is distinguishable from another population of microbeads based on the metal or the plurality of metals of the microbeads; binding different sample biomolecules of the sample to the different biomolecules of distinct populations of microbeads; binding, directly or indirectly, a reporter to each of the different sample biomolecules, wherein the reporter bound to each of the different sample biomolecules comprises a mass tag; and detecting the metal and the mass tag of individual microbeads by mass spectrometry.

Such a method may further include attaching the different biomolecules to the microbeads of the distinct populations of microbeads prior to the step of mixing the distinct populations of microbeads with the sample. One or more of the different sample biomolecules may include oligonucleotides, antibodies (or another affinity reagent), cytokines, cancer biomarkers (such as one or more prostate specific antigens) etc.

In some embodiments, a method of mass spectrometry analysis uses a kit of any of the embodiments described herein, and further includes the steps of: mixing distinct populations of microbeads with a sample, wherein microbeads of each population of microbeads are bound to a different biomolecule and wherein each population of microbeads is distinguishable from another population of microbeads based on the metal or the plurality of metals of the microbeads; binding different sample biomolecules of the sample to the different biomolecules of distinct populations of microbeads; binding, directly or indirectly, a reporter to each of the different sample biomolecules, wherein the reporter bound to each of the different sample biomolecules comprises a mass tag; and detecting the metal and the mass tag of individual microbeads by mass spectrometry.

In some embodiments, a kit or method of mass spectrometry analysis of a cellular sample using the microbeads of any embodiment described herein includes the steps of: providing a plurality of mass-tagged antibodies, wherein each antibody of the mass tagged antibody is conjugated to a polymer mass tag chelating multiple atoms of a metal or enriched isotope thereof; contacting a sample with the plurality of mass-tagged antibodies; detecting the mass-tagged antibodies bound to the sample and the microbeads by mass spectrometry. Microbeads and the cellular sample may be combined prior to analysis by mass spectrometry, or may be analyzed in separate sample runs. The microbeads may be used to assay one or more sample biomolecules. Alternatively or in addition, microbeads may be an element standard used for calibration, normalization and/or quantitation as described further herein.

The microbeads may be of a single population having a consistent size and/or amount of one or more metals as described herein. Alternatively, the microbeads may be of distinct populations each characterized by a different set of metals, combination of metals, and/or amount of metals. For example, a set of microbead populations that together have metals from more than 10 distinct elements (e.g., more than 30 distinct isotope masses). For example, the microbeads of the subject application may together provide signal in more than 4, more than 6, more than 10, more than 20, or more than 30 mass channels (e.g., atomic mass channels greater than 80 amu). Microbeads may be added alongside cells in a suspension mass cytometry workflow or may be provided on the same support as a tissue section or cell smear in an imaging mass cytometry workflow. Microbeads may be used in an assay as described herein, or may be a standard (e.g., that provides a signal in most or all mass channels that a mass tag detected by mass cytometry is detected in). The mass spectra of microbeads that have different amounts of the same metal (e.g., or enriched isotope thereof) may be used to create a curve (e.g., of a signal intensity to a known amount of metal) used in calibration, normalization or quantitation as described further herein.

Microbeads may be used for calibrating the mass spectrometer used in the step of detecting of any method described herein. Calibration may be of one or more of mass resolution, mass calibration, dual count calibration, pre-xy and xy optimization, detector voltage, gas calibration or current calibration. Mass Resolution ensures that there is sufficient separation between ions of different mass and may be based in part of the shape of a peak from a specific isotope. A mass resolution above a certain value may indicate a pass. Mass Calibration may include auto-tuning that checks the values of one or more mass channels (e.g., from a metal of microbead standard) and then calculates the TOF values for additional mass channels and/or may include aligning the correct ions to the detection channel so that the entire signal for each ion is collected. Dual Count Calibration may determine dual count coefficients (dual slopes to correlate pulse count and intensity (the dual count coefficient converts analog signal to ion count signal). This correlation may be important when ion concentrations increase and pulses overlap, for example, during a cell or microbead event. XY optimization is the process by which the optimal alignment of the torch with the vacuum interface is determined to provide the maximum signal for a mass channel (e.g., from a metal of microbead standard). Optimizing the alignment of the system is important for maximum transmission of ions into the vacuum interface. Detector voltage calibration uses the dual count calibration to determine the detector voltage that provides the best signal while ensuring the longevity of the detector. The optimum detector voltage may be achieved when the dual count coefficient is 0.03±0.003. The detector voltage should not be more positive than-1,100 V. Gases and/or current calibration may optimize the nebulizer gas flow and the makeup gas flow (and optionally additional gas flows) using the maximum mass channel signal (e.g., from a metal of microbead standard) that can be achieved by varying the makeup gas flow and nebulizer gas flow while controlling oxide formation. This may ensure that the plasma temperature is optimal in the system and minimal metal oxides are formed. The current that is applied at the vacuum interface is increased in increments to drive the transfer of the ion cloud through the interface. The value that provides the highest signal may then be selected. Calibration may be performed during a sample run (e.g., to account for sensitivity drift).

Microbeads may be used as a standard for normalizing a mass spectrometry signal obtained from the mass tags (e.g., mass tags of a sample as described herein) based on a mass spectrometry signal obtained from the microbeads (e.g., obtained from microbeads comprising the one or more of the same metals as at least one of the mass tags, or similar mass spectrum, or standard curve created from a plurality of microbead populations that comprise different amounts of a metal). Each of the tags may provide a signal in one or more mass channels. Such normalization of mass tag signal may be for individual cell or assay microbead events (e.g., in suspension mass cytometry) or individual cells, assay microbeads or pixels (e.g., in imaging mass cytometry). For example, individual cells of a cell smear may be detected by IMC, or cells of a solid tissue section segmented algorithmically based on a membrane stain, and the mass tag signal across the single cell may be normalized or quantified as described herein. The mass tag signals of a cell or pixel may be normalized to signal from microbeads that are detected within a time interval of the cell or pixel, such as within 10,000 seconds, within 5,000 seconds, within 2,000 seconds, within 1,000 seconds, within 500 seconds, within 200 seconds, or within 100 seconds of when the cell or pixel was detected. Alternatively or in addition, if the microbeads used as a standard include microbeads of distinct populations that have different metals, normalization of a mass tag signal may be based on one or more microbeads whose metals are detected in the same mass channel as the mass tag. Alternatively or in addition, if the microbeads used as a standard include microbeads of distinct populations that have different amounts of the metal, normalization of a signal from a mass tag comprising the same metal may be based on one or more microbeads whose that provide a similar signal intensity (e.g., at or less than a ten fold difference, at or less than a five fold difference, at or less than a two fold difference, etc.) for the same mass channel. As described herein, a metal may be an enriched isotope. As such, a microbead standard comprising population of microbeads having different metals and/or amounts of metals may be used. Such normalization may be similar to the use of EQ4™ beads provided by Fluidigm to normalize mass cytometry data (e.g., normalize data of FCS files obtained by mass cytometry). However, the microbeads of the present disclosure may together provide signal more than 4, more than 6, more than 10, more than 20, or more than 30 mass channels (e.g., atomic mass channels greater than 80 amu).

Microbeads may be used as a standard for quantitating the amount of one or more mass-tagged antibodies (or other mass-tagged biomolecules), such as when a known (or estimated) number of metal atoms from the mass tag are associated with the antibody (or other biomolecule). For example, the number of metal atoms associated with a mass-tagged antibody (or other biomolecule) that may have more than one instance of the mass tag may be determined by: starting from a known number of metals per mass tag (e.g., on a polymer mass tag) and UV/visible spectrum or ICP-MS signal characteristic; and further analyzing fractions of the mass-tagged antibody (or other biomolecule) by UV or Visible spectrum or ICP-MS. Such analysis could be done on fractions obtained by fast protein liquid chromatography. Quantifying the mass-tagged antibodies may therefore be based on the average number of metal atoms for each of the mass-tagged antibodies and the detected mass spectrometry signal from the mass-tagged antibodies and the microbeads. The microbeads may have a known number of metal atoms (e.g., determined as described herein). The microbeads and the mass-tagged antibodies (or other biomolecule) may include the same metals. As such, quantifying the mass-tagged antibodies may be calculated as the number of a metal in a microbead, times the ratio of a signal from a mass-tagged antibody (or other biomolecule) to the signal of the metal from the microbead, and divided by the average number of metal atoms per antibody (or other biomolecule). Quantitation may be performed for individual cells, assay microbeads, or pixels.

Embodiments of the present disclosure include a computer readable medium configured to perform one or more of calibration, normalization, or quantitation as described herein.

In some embodiments, a step of detecting may include inductively coupled plasma mass spectrometry (ICP-MS), such as for suspension mass cytometry or imaging mass spectrometry. Wherein the mass spectrometry may be by a simultaneous mass spectrometry, such as time-of-flight mass spectrometry (TOF-MS) or magnetic sector mass spectrometry.

In some embodiments, detecting the microbeads may be by imaging mass spectrometry (e.g., imaging mass cytometry). The microbeads (e.g., a microbead standard) may be fused (e.g. melted) to a solid surface prior to the step of detecting. Imaging mass spectrometry may be, for example, by laser ablation ICP-MS or by secondary ion mass spectrometry (SIMS).

A biological sample can include any sample of a biological nature that requires analysis. For example, samples may include biological molecules, tissue, fluid, and cells of an animal, plant, fungus, or bacteria. They may also include molecules of viral origin. Typical samples include, but are not limited to, sputum, blood, blood cells (e.g., PBMCs), tissue or fine needle biopsy samples, urine, peritoneal fluid, and pleural fluid, or cells therefrom. Biological samples may also include sections of tissues such as frozen sections taken for histological purposes. Another typical source of biological samples are viruses and cell cultures of animal, plant, bacteria, fungi where gene expression states can be manipulated to explore the relationship among genes. In some cases, other samples can be interrogated, such as artificial samples. Certain aspects of the present disclosure are especially useful when interrogating samples of a human origin, and especially useful when interrogating samples of human peripheral blood.

Mass tagged oligonucleotides may be hybridized, directly or indirectly, to a target oligonucleotide. For example, one or more intermediate oligonucleotides may provide a scaffold on which a plurality of mass tagged oligonucleotides can hybridize, thereby amplifying signal. Aspects of the subject application therefore include oligonucleotides for hybridization based signal amplification.

In some aspects, a sample biomolecule may be a target oligonucleotide, such as a DNA or RNA molecule (such as coding RNA, small interfering RNA, or micro RNA) of a cell or bead. The target oligonucleotide may be single stranded. The target oligonucleotide may have a known specific sequence (or homology to a known specific sequence).

In some aspects, a reporter may include a non-oligonucleotide biomolecule, such as an antibody or derivative thereof, which may be conjugated to an oligonucleotide, such as to a synthetic single stranded DNA oligonucleotide comprising a known sequence. In such cases, both the antibody and the oligonucleotides may be referred to as a part of a reporter.

The signal from a target oligonucleotide, or a reporter comprising a non-oligonucleotide biomolecule conjugated to an oligonucleotide, may be amplified through a hybridization scheme. The hybridization may be branched or linear. In certain aspects, a polymerase may extend the first oligonucleotide along a template to provide additional sites for attachment of an element tag (such as additional hybridization sites for an element tagged oligonucleotide). Mass tagged oligonucleotides may include a single labeling atom, or may include a polymer comprising multiple labeling atoms, and may be referred to as a reporter. Mass tagged oligonucleotides may include a labeling atom, such as a heavy metal atom, in the chemical structure of the oligonucleotide itself.

Signal amplification may uniquely benefit bead-based assays, in which the same reporter tag (labeling metal element or isotope) can be amplified and used across different beads and their target analytes.

In cases where the assay biomolecule is an oligonucleotide, an element tagged reporter oligonucleotide may hybridize to another portion of the target RNA or DNA, thereby providing a signal when the target RNA or DNA is bound to the bead. In cases where the assay biomolecule is an affinity reagent, such as an antibody that binds an analyte at a first epitope, an element tagged reporter affinity reagent (e.g., reporter antibody) may bind to another epitope on the analyte, thereby providing a signal when the target analyte is bound to the bead. The analyte may further be bound by a reporter, such as an element tagged reporter antibody or oligonucleotide. The reporter may comprise a high sensitivity (e.g., intensity) element tag that provides a highly abundant isotope (e.g., more than 50, 100, 200, 500, 1000 copies of a single isotope), thereby enabling detection of a smaller number of a target analyte bound to an assay bead. Such a high sensitivity element tag may include a nanoparticle (e.g., comprising a metal nanocrystal surface functionalized to bind biomolecule such as an antibody or oligonucleotide) or a hyperbranched polymer. For example, multiple reporter biomolecules comprising the same nanogold particle element tag would provide a high signal and take advantage of the fact that separate reporter biomolecules comprising the same element tag can be distinguished by the assay barcode (e.g., of the bead presenting the analyte they are specific to). In certain aspects, a nanoparticle tag (e.g., gold nanoparticle) may be associated with a reporter probe through a biotin-avidin (e.g., biotin-streptavidin) interaction. For example, the nanoparticle (e.g., gold nanoparticle) may be conjugated to streptavidin. The reporter may also comprise a low sensitivity element tag that provides a low abundance isotope (e.g., less than 100, 50, 30, 20, 10, or 5 copies of an isotope) that is distinct from the highly abundant isotope, thereby allowing detection/quantitation of the amount of an analyte that is such high abundance that the highly abundant isotope would saturate the detector. In certain aspects, the high and low abundance isotopes have a difference in mass (e.g., greater than 5, 10, 20, 30, 40 or 50 amu) such that saturation of the detector by the high abundance isotope does not affect detection of the low abundance isotope. Reporters for different analytes (e.g., comprising antibodies that bind to target analytes of different assay beads) may comprise the same isotopes or combination of isotopes, since the analytes will be distinguished by the unique assay barcodes of the beads.

In certain aspects, a reporter may include a reporter system that provides signal amplification through association of a plurality of instances of an element tag with a single instance of the target analyte (e.g. sample biomolecule). Signal amplification may be by enzymatic deposition, hybridization (e.g., branched hybridization, chain hybridization, and/or hybridization of a plurality of reporter oligonucleotides to a single long intermediate oligonucleotide), extension (e.g., a single extension, rolling circle extension), and/or a series of branched conjugations. In certain aspects, a plurality (e.g., all) of the analytes detected with the assay beads may be detected with the same reporter system. In certain aspects, a signal amplification reporter system may have a high sensitivity element tag.

For example, an element tag comprising an enzyme substrate moiety may be deposited from solution onto the bead (or a molecule attached to the bead) by an enzyme attached to a reporter biomolecule. Such a reaction may be covalent binding by a tyramide element tag acted on by horse radish peroxidase bound to a reporter biomolecule.

Aspects include a hybridization scheme, such that a plurality of element tagged oligonucleotides hybridize indirectly (through one or more oligonucleotide intermediates) to a single oligonucleotide target. For example, the oligonucleotide target may be a target RNA or DNA (e.g., gDNA or cDNA) sequence, or may be an oligonucleotide present on a reporter antibody.

As described herein, mass cytometry may enable enough detection channels (mass channels) to detect both a sample and assay barcode in beads while allowing an additional channel for a reporter (e.g., for detecting the assay target). As such, the bead assays described herein may be sample and/or assay barcoded for use in mass cytometry. For example, a plurality of different conditions (e.g., drug candidates such as enzymes or an agonist or antagonist of one or more enzymes) may be applied to a biological sample, and its effect on a plurality of targets may be detected with enzyme assay beads. Individual conditions may be identified with a sample barcode shared across different assay beads exposed to the same conditions. Assay barcoded beads may be combined prior to analysis, such as before exposure to a condition. Sample barcoded beads may be combined prior to analysis.

In certain aspects, the enzyme may be a protease, kinase, phosphatase, or a DNA modifying protein such as a DNA methyltransferase. The target may be the substrate acted on by the enzyme, and the reporter (e.g., a reporter biomolecule as described herein) may only bind the target (e.g. sample biomolecule) before or after it is acted on by the enzyme. For example, a phospho-specific antibody that detects the phosphorylated form of a protein target, which may be increase in abundance when acted on by a kinase enzyme or decreased in abundance when acted on by a phosphatase. When the enzyme is a protease, a reporter may be associated with the end of a peptide substrate and removed from association with the bead when the substrate is cleaved (such that a decrease in the reporter element tag indicates an increase in protease activity).

Sample barcode may be used to indicate which of a number of enzymes (or agonists/antagonists thereof) was tested in a particular assay. For example, the candidate enzyme, agonist, antagonist, may be added to a biological fluid such as a cell lysate, after which the sample is contacted with assay barcoded beads to detect activity of the enzyme. Alternatively, the candidate may be administered to cells (e.g., directly or by genetic engineering) or to an organism such as a patient or mammalian test subject, and a sample taken from that source may be contacted with the assay beads. Sample barcoding allows many such candidates to be screened in parallel. In either case, the sample barcode can be added as described for beads herein to identify the candidate. For example, more than 10, more than 20, more than 50, more than 100, more than 500, or more that 1000 distinct samples can be barcoded. For example, 12 distinct isotopes in unique combinations of 6 provides 924 distinct combinations (e.g., for barcoding of up to 924 samples). Another 12 distinct isotopes could be used to barcode close to 1000 assays. As such, more than 10, more than 20, more than 50, more than 100, more than 500, or more that 1000 distinct assay beads can be barcoded (e.g., beads detecting amount of a different substrate acted on by the candidate). At least one channel would be left for detection of the substrate by a reporter, as described herein. This may allow unprecedented screening with an immediate readout by mass cytometry.

Post-translational modifications of proteins are carried out by enzymes within living cells. Known post-translational modifications include protein phosphorylation and dephosphorylation as well as methylation, prenylation, sulfation, and ubiquitination. The presence or absence of the phosphate group on proteins, especially enzymes, is known to play a regulatory role in many biochemical pathways and signal transduction pathways.

Bead based kinase assays for mass cytometry are discussed in US patent publication US20070190588, which is incorporated by reference and summarized below. However, such bead based assays have not been proposed for both sample and assay barcoding, which provides advantages for screening and is uniquely enabled by the high plexity of mass cytometry.

A kinase function is to transfer phosphate groups (phosphorylation) from high-energy donor molecules, such as ATP, to specific target molecules (substrates). An enzyme that removes phosphate groups from targets is known as a phosphatase. The largest group of kinases are protein kinases, which act on and modify the activity of specific proteins. Various other kinases act on small molecules (lipids, carbohydrates, amino acids, nucleotides and more) often named after their substrates and include: Adenylate kinase, Creatine kinase, Pyruvate kinase, Hexokinase, Nucleotide diphosphate kinase, Thymidine kinase.

Protein kinases catalyze the transfer of phosphate from adenosine triphosphate (ATP) to the targeted peptide or protein substrate at a serine, threonine, or tyrosine residue. Protein kinases are distinguished by their ability to phosphorylate substrates on discrete sequences. Commercially available kinases can be in the active form (phosphorylated by supplier) or in the inactive form and require phosphorylation by another kinase.

A protein phosphatase hydrolyses phosphoric acid monoesters at phosphoserine, phosphothreonine, or phosphotyrosine residue into a phosphate ion and a protein or peptide molecule with a free hydroxy group. This action is directly opposite to that of the protein kinase. Examples include: the protein tyrosine phosphatases, which hydrolyse phospho tyrosine residues, alkaline phosphatase, the serine/threonine phosphatases and inositol monophosphatase.

Another aspect of the present disclosure is to provide a kit for the detection and measurement of elements in a sample, where the measured elements include an element tag attached to a phosphorylated substrate, an element of a metal ion coordination complex, and elements of uniquely labeled supports, comprising: an element tag for directly tagging phosphorylated substrate; a multitude of phosphorylated substrates; uniquely labeled supports; metal ion coordination complex; and optionally, phosphatase, phosphatase buffer and ADP.

Another aspect of the subject disclosure is to provide a method for a kinase assay, comprising: incubating ATP, at least one kinase, a free metal ion coordination complex, and a multitude of non-phosphorylated substrates immobilized on element labeled supports in such manner that a single type of non-phosphorylated substrate is attached to a single type of element labeled support, in conditions to enable the kinase to phosphorylate the substrates; separating the multitude of phosphorylated substrates immobilized on element labeled supports having attached metal ion coordination complex from the free metal ion coordination complexes and the multitude of immobilized non-phosphorylated substrates; and measuring the multitude of phosphorylated substrate immobilized on element labeled supports having attached metal ion coordination complex by elemental analysis.

Another aspect of the subject disclosure is to provide a kit for the detection and measurement of elements in a sample, where the measured elements include an element tag attached to a non-phosphorylated substrate and a metal ion coordination complex, comprising: an element tag for directly tagging non-phosphorylated substrate; non-phosphorylated substrate; a solid support; a metal ion coordination complex; and optionally, kinase; kinase buffer; and ATP.

Pharmacological regulation of enzymes has become key elements in identifying possible therapeutic agents. Proteases are a subclass of protein-degrading enzymes that have recently been shown to play a vital role in signaling pathways, the dysregulation of which can result in cancer, cardiovascular disease, and neurological disorders. Of the approximately 400 known human proteases, dozens are being studied as potential drug candidates. Small-molecule inhibitors of proteases are now considered valuable therapeutic leads for the treatment of degenerative diseases, for the treatment of cancer, and as antibacterials, antivirals and antifungals. Bead based protease assays for mass cytometry are discussed in US patent publication US20170023583, which is incorporated by reference and summarized below. However, such bead based assays have not been proposed for both sample and assay barcoding, which provides advantages for screening and is uniquely enabled by the high plexity of mass cytometry.

There is a need for a robust, sensitive, and quantitative enzyme assay that allows for simultaneous measurement of multiple enzymatic reactions. Such an assay can allow for conservation of valuable biological sample and reagents, achieve high-throughput and reduced assay time, and decrease the overall cost of enzyme analysis.

One aspect of the invention is a method for detecting protease activity in a biological fluid. The method may include attaching a coded bead to a first amino acid of a peptide substrate to form an immobilized peptide substrate, the peptide substrate comprising a first amino acid and a last amino acid and being a substrate for a protease enzyme: attaching an element tag to the last amino acid of the peptide substrate to form a tagged peptide substrate: incubating the immobilized, tagged peptide substrate with the biological fluid; and detecting the element tag and the coded bead in the biological fluid by elemental analysis.

An encoded microbead may be both assay and sample barcoded, as discussed herein.

A protease assay kit may include an assay coded bead attached to a first amino acid of a peptide substrate (an immobilized peptide substrate), the peptide substrate may include a first amino acid and a last amino acid and may be a substrate for a protease enzyme. An element tag may be attached at or near the last amino acid of the peptide substrate to form a tagged peptide substrate. The coded bead may be both assay and sample barcoded, as discussed herein.

A mixture of assay beads may together target at least 5, 10, 20, 50, 100, 200, 500, 1000 or more analytes (sample biomolecules). In certain aspects, sample barcodes may distinguish assay barcode beads and/or cells from at least 5, 10, 20, 50, or 100 or more different samples.

Sample barcoding reagents for cells may include an element-tagged antibody or antibodies (that bind across a plurality of cell types or majority of cells in the sample), an element tag functionalized to bind non-specifically to cells (e.g., through a covalent interaction), and/or metal in solution. Sample barcoding reagents for cells may further include a reagent for bringing the sample barcode into the cell (e.g., DMSO, cell permeabilization reagents such as a detergent or alcohol, etc.). Sample barcoding reagents for assay barcoded beads may be present within the beads, on the surface of the beads, or may be applied to the beads. If for application to beads, the sample barcoding reagent may comprise functional groups as described herein to bind to the surface of the bead (e.g., to bind to functional groups presented by the bead or to a blocking reagent present on the bead surface). Sample barcoding reagents for a given sample may comprise a unique combination of isotopes specific to that sample. In certain aspects, cells and assay barcoded beads from the same sample (e.g., an individual blood sample) may be labeled with the same assay barcode. The same assay barcode used for labeling of cells and beads may comprise the same combination of isotopes and/or same means of attachment (e.g., functional group).

Sample barcoding reagents may be provided in admixture or alongside with an antibody panel, such as a lyophilized antibody panel. Sample barcoding reagents may be provided in admixture or alongside with assay barcoded beads. Assay barcoded beads may be provided in admixture or alongside an antibody panel. Assay barcoded beads and sample barcoding reagents may be provided in admixture with or alongside a lyophilized antibody panel (e.g., where the sample barcoding reagents bind both assay barcoded beads and cells in the sample). In certain embodiments above, sample barcoding reagents may be in, on, or provided alongside sample and/or assay barcoded beads.

In some cases, barcoding reagents can be provided in a pre-configured form by preparing the barcoding reagents with a number of unique combinations of assay barcodes and sample barcodes. In such cases, each unique barcoding reagent can be stored in distinct containers, such as distinct wells of a well plate. In an example, a well plate can be established such that all wells along a particular column (or row) share the same assay barcode, whereas all wells along a particular row (or column) share the same sample barcode. In another example, a well plate can be established such that each filled well contains barcoding reagents with various combinations of a particular unique sample barcode and numerous assay barcodes. Thus, a first well may contain barcoding reagents all having a first sample barcode but each having different assay barcodes, and a second well may contain barcoding reagents all having a second barcode but each having different assay barcodes. In some cases, pre-configured barcoding reagents can require the manufacture of thousands of groups of unique beads.

In some cases, barcoding reagents (e.g., beads) can be provided in a semi-configured form by preparing the barcoding reagents with unique assay barcodes and a surface functionalized to bind a sample barcode. In such cases, each group of barcoding reagents can be coupled to a biomolecule (e.g., antibody) having a targeting function associated with the assay that is associated with the assay barcode of that group of barcoding reagents.

When semi-configured barcoding reagents are provided, the sample barcodes can be bound to the barcoding reagents before combining the barcoding reagents with samples. In an example, the different barcoding reagents can be mixed together and then placed across a set of containers (e.g., wells in a well plate). Then, unique sample barcodes can be added to each of the containers, the result of which can be mixed with a unique sample to perform assay-barcode-identifiable assays on that sample and simultaneously tag that sample with the sample barcode.

When semi-configured barcoding reagents are provided, the sample barcodes can be bound to the barcoding reagents after combining the barcoding reagents with samples. In an example, the semi-configured barcoding reagents can be provided together or otherwise mixed together. Then, the barcoding reagents can be added to each of a set of samples. Separately, before or after the barcoding reagents are added, a unique sample barcode can be mixed with each of the set of samples. The sample barcode can tag the barcoding reagents and/or the cells or particles of the sample.

In an example case, barcoding reagents can include assay barcoded beads functionalized with poly dopamine for the attachment of capture antibodies. Another molecule (e.g., avidin) can be added alongside the capture antibodies. After capture antibodies are added to the assay barcoded beads, the beads could be mixed and split into an aliquot for each sample. For the sample barcode, a unique combination of element tags functionalized (e.g., with biotin) to bind the molecule can be added.

In some cases, elemental analysis can be conducted on an individual particle basis, known as particle elemental analysis. Particle elemental analysis includes determining the elemental composition of individual particles (e.g., cell-by-cell), such as using a mass spectrometer-based flow cytometer. Certain aspects of the present disclosure make use of particle elemental analysis on a cell-by-cell basis, which can be known as cytometric elemental analysis. In some cases, elemental analysis can be conducted on a bulk basis, known as bulk elemental analysis or solution elemental analysis. Bulk elemental analysis includes determining the elemental composition of the entire volume of a sample.

Elemental analysis can be used to interrogate a sample, such as a biological sample. If the sample is labelled with a known element tag, detection of the element tag during elemental analysis can be indicative of characteristics of the sample associated with the element tag.

As referred to herein, mass cytometry is any method of detecting element tags (mass tags) in a biological sample, such as simultaneously detecting a plurality of distinguishable mass tags with single cell resolution. Mass cytometry may include analysis of mass tagged beads, separate from or in addition to cells. Any of the subject kits and methods may include or be adapted to mass cytometry. Mass cytometry includes suspension mass cytometry and imaging mass cytometry (IMC).

Suspension mass cytometry includes analysis of suspended element tagged cells and/or beads by mass spectrometry (e.g., by atomic mass spectrometry), and is described in US patent publications including US20050218319, US20150183895, US20150122991, all of which are incorporated by reference herein.

Imaging mass cytometry (IMC) includes any imaging mass spectrometry (e.g., imaging atomic mass spectrometry) of element tagged biological sample, such as a tissue section or cell smear. IMC may atomize and ionize mass tags of a cellular sample by one or more of laser radiation, ion beam radiation, electron beam radiation, and/or inductively coupled plasma (ICP). Mass cytometry may simultaneously detect distinct mass tags from single cells, such as by time of flight (TOF) or magnetic sector mass spectrometry (MS). Examples of mass cytometry include suspension mass cytometry where cells are flowed into and ICP-MS and imaging mass cytometry where a cellular sample (e.g., tissue section) is sampled, for example by laser ablation (LA-ICP-MS) or by a primary ion beam (e.g., for SIMS). Laser based IMC is described in US patent publications US20160131635, US20170148619, US20180306695, and US20180306695 all of which are incorporated by reference herein. In certain aspects, when the sample is a cell smear for analysis by IMC, the cells may be processed as described herein such as by staining with a lyophilized panel, sample barcoding, and/or assay barcoding. Similarly, assay beads described herein may be analyzed separately or in mixture with cells, by IMC.

Mass tags may be sampled, atomized and ionized prior to elemental analysis. For example, mass tags in a biological sample may be sampled, atomized and/or ionized by radiation such as a laser beam, ion beam or electron beam. Alternatively or in addition, mass tags may be atomized and ionized by a plasma, such as an inductively coupled plasma (ICP). In suspension mass cytometry, whole cells including mass tags may be flowed into an ICP-MS, such as an ICP-TOF-MS. In imaging mass cytometry, a form of radiation may remove (and optionally ionize and atomize) portion (e.g., pixels, region of interest) of a solid biological sample, such as a tissue sample, including mass tags. Examples of IMC include LA-ICP-MS and SIMS-MS of mass tagged sample. In certain aspects, ion optics may deplete ions other than the isotope of the mass tags. For example, ion optics may remove lighter ions (e.g., C, N, O), organic molecular ions. In ICP applications, ion optics may remove gas such as Ar and/or Xe, such as through a high-pass quadrupole filter. In certain aspects, IMC may provide an image of mass tags (e.g., targets associated with mass tags) with cellular or subcellular resolution.

The present disclosure also provides the following embodiments:

Embodiment 1. A metal-encoded microbead comprising:
a copolymer comprising:
   a structural monomer, and
   a metal-chelating monomer comprising a metal and a chelator;
   wherein the chelator coordinates the metal at least at 3 sites; and
   wherein the structural monomer does not comprise the chelator.

Embodiment 2. The microbead of embodiment 1, wherein the structural monomer is selected from substituted or unsubstituted styrene, alpha-methylstyrene, acrylic acid and esters and amides thereof, methacrylic acid and esters and amides thereof, and derivatives thereof, optionally the structural monomer is styrene.

Embodiment 3. The microbead of embodiment 1 or 2, wherein the metal-chelating monomer has a structure of Formula I prior to polymerization

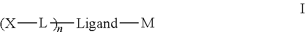

wherein Ligand is the chelator, L is a linker, X is a polymerizable end group, M is the metal, and n is 1 or an integer greater than 1, wherein the metal-chelating monomer is neutral in charge prior to polymerization.

Embodiment 4. The microbead of embodiment 3, wherein L is selected from a bond, C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O) O, or mixtures thereof, optionally each of the alkylene, aryl, alkylaryl, alkylheteroaryl, cycloalkyl, cycloalkylaryl, and cycloalkylheteroaryl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

Embodiment 5. The microbead of embodiment 3 or 4, wherein L is attached to the chelator through an amide or an ester.

Embodiment 6. The microbead of any one of embodiments 3 to 5, wherein the polymerizable end group is selected from arylvinyl, styrene, alpha-methylstyrene, acrylate ester, methacrylate ester, acrylamide, 2-methylacrylamide, and mixtures thereof, optionally the polymerizable end group is arylvinyl or styrene.

Embodiment 7. The microbead of any one of embodiments 1 to 6, wherein the chelator is tetradentate, pentadentate, hexadentate, heptadentate, or octadentate, optionally the chelator is hexadentate or octadentate.

Embodiment 8. The microbead of any one of embodiments 1 to 7, wherein the chelator comprises an aminopolyacid moiety, or a derivative thereof.

Embodiment 9. The microbead of embodiment 8, wherein the aminopolyacid moiety is selected from aminopolycarboxylic acid, aminopolyphosphonic acid, or combinations thereof.

Embodiment 10. The microbead of embodiment 8 or 9, wherein the aminopolyacid moiety is a substituted oligomer of one or more of ethylene imine, propylene amine, or mixtures thereof, the oligomer being substituted with two or more carboxylic acids and/or phosphonic acids, optionally the oligomer is a crown ether or an aza-crown ether.

Embodiment 11. The microbead of embodiment 10, wherein the oligomer is further substituted with one or more substituents selected from C1-C6 alkyl, C1-C6 alkenyl, C3-C8 cycloalkyl, C3-C8 heterocycloalkyl, amide, ester, aryl, heteroaryl, akylaryl, alkylheteroaryl, C3-C8 cycloalkylaryl, C3-C8 cycloalkylheteroaryl, CN, or mixtures thereof.

Embodiment 12. The microbead of any one of embodiments 1 to 11, wherein the chelator is selected from DFO, EDTA, DTPA, EGTA, EDDS, EDDHA, BAPTA, H4neunpa, H6phospa, H4CHXoctapa, H4octapa, H2CHXdedpa, H5decapa, Cy-DTPA, Ph-DTPA, a TACN-type chelator, a TACD-type chelator, a cyclen-type chelator, a cyclam-type chelator, a (13) aneN4-type chelator, a 1,7-diaza-12-crown-4-type chelator, a 1,10-diaza-18-crown-6-type chelator, or derivatives thereof.

Embodiment 13. The microbead of embodiment 12, wherein the TACN-type chelator is selected from NOTA, NOPO, TRAP, or derivatives thereof.

Embodiment 14. The microbead of embodiment 12, wherein the cyclen-type chelator is DOTA or derivatives thereof.

Embodiment 15. The microbead of embodiment 12, wherein the cyclam-type chelator is selected from TETA, cross bridged-TETA, DiAmSar, or derivatives thereof.

Embodiment 16. The microbead of embodiment 12, wherein the (13) aneN4-type chelator is selected from TRITA or derivatives thereof.

Embodiment 17. The microbead of embodiment 12, wherein the 1,10-diaza-18-crown-6-type chelator is selected from MACROPA, or derivatives thereof.

Embodiment 18. The microbead of embodiment 12, wherein the chelator is selected from DTPA, Cy-DTPA, Ph-DTPA, or derivatives thereof.

Embodiment 19. The microbead of embodiment 18, wherein the derivative of DTPA comprises DTPA where two adjacent carbon atoms are joined together with atoms therebetween to form a 5-membered or 6-membered ring, optionally a cycloalkyl ring, an aryl or a heteroaryl ring.

Embodiment 20. The microbead of embodiment 18, wherein prior to polymerization, the metal-chelating monomer is

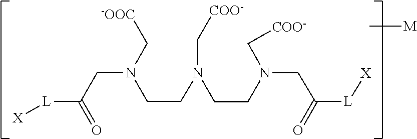

wherein L and X are as defined in any one of embodiments 4 to 6.

Embodiment 21. The microbead of embodiment 20, wherein the metal-chelating monomer is selected from

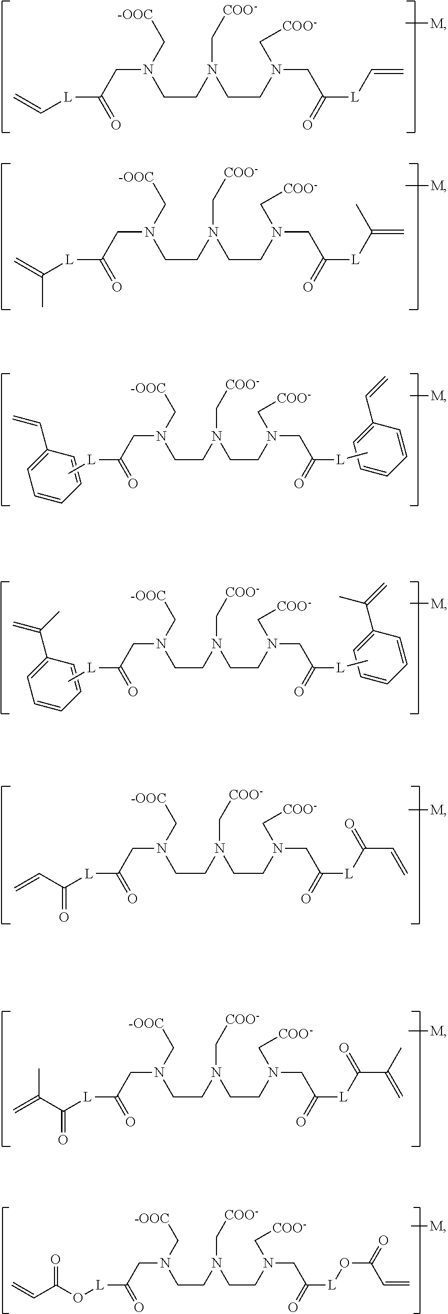

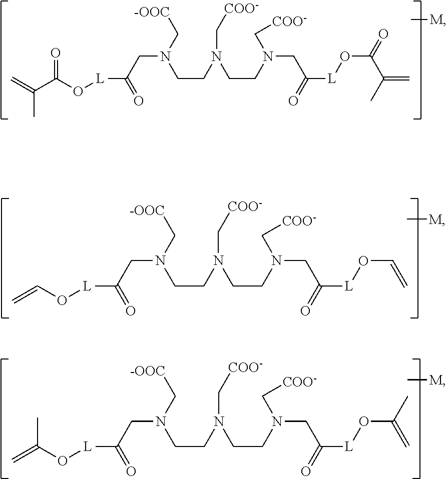

or mixtures thereof.

Embodiment 22. The microbead of any one of embodiments 1 to 9, wherein the chelator comprises porphyrin or phthalocyanine.

Embodiment 23. The microbead of embodiment 22, wherein the chelator is substituted or unsubstituted porphyrin.

Embodiment 24. The microbead of embodiment 22 or 23, wherein the metal-chelating monomer prior to polymerization is selected from

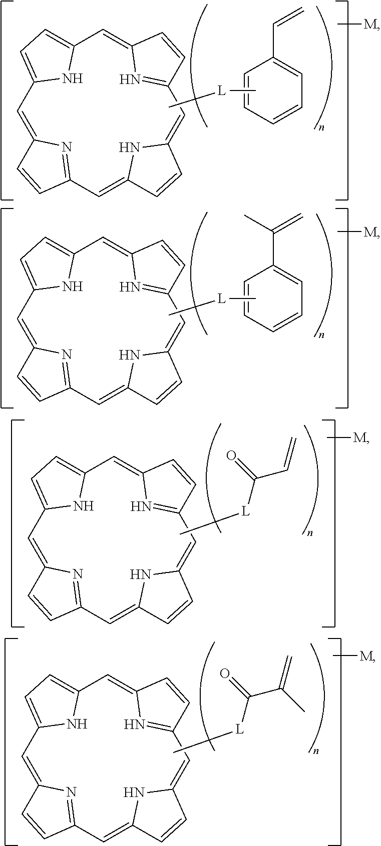

or mixtures thereof, and wherein n is an integer from 1 to 4.

Embodiment 25. The microbead of embodiment 24, wherein L is aniline.

Embodiment 26. The microbead of embodiment 24 or 25, wherein n is at least 2.

Embodiment 27. The microbead of embodiment 3, wherein the metal-chelating

monomer is selected from

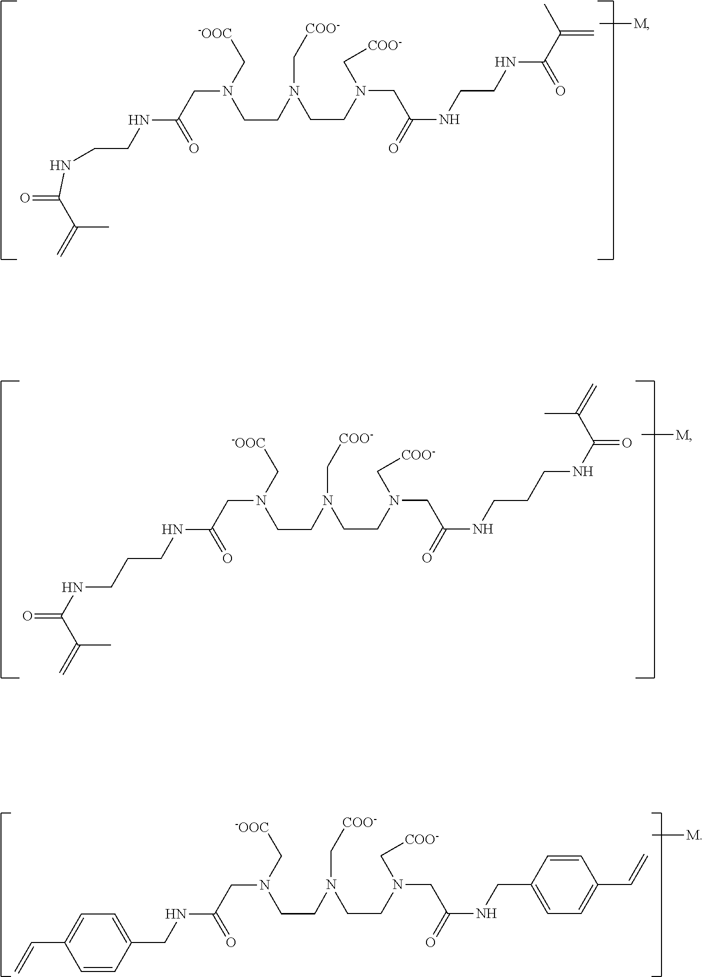

Embodiment 28. The microbead of any one of embodiments 1 to 27 further comprising a steric stabilizer, optionally the steric stabilizer is selected from PVP, polyvinyl alcohol, hydroxymethylcellulose, hydroxyethylcellulose, hydroxypropylcellulose, polyacrylic acid, water soluble homopolymers of acrylic acid esters, water soluble homopolymers of methacrylic acid esters, water soluble homopolymers of acrylamide, homopolymers of methacrylamide, water soluble copolymer steric stabilizers, or mixtures thereof.

Embodiment 29. The microbead of embodiment 28, wherein the water soluble copolymer steric stabilizer is selected from copolymers of acrylic acid ester, methacrylic acid ester, acrylamides or methacrylamide with methyl acrylate and/or ethyl acrylate, or mixtures thereof.

Embodiment 30. The microbead of any one of embodiments 1 to 29, wherein the copolymer is crosslinked.

Embodiment 31. The microbead of any one of embodiments 1 to 30, wherein the metal is a plurality of metals.

Embodiment 32. The microbead of embodiment 31, wherein the plurality of metals comprises one or more enriched isotopes.

Embodiment 33. The microbead of embodiment 31 or 32, wherein the plurality of metals comprises at least 2 metals, at least 3 metals, or at least 4 metals.

Embodiment 34. The microbead of any one of embodiments 31 to 33, wherein the amount of each metal of the plurality of metals is within about 20% or about 10% of the amount of another metal of the plurality of the metals.

Embodiment 35. The microbead of any one of embodiments 1 to 34, wherein the metal comprises indium, bismuth, or a rare earth metal, optionally the rare earth metal is selected from lanthanide metal, yttrium, or mixtures thereof.

Embodiment 36. The microbead of embodiment 35, wherein the metal comprises a rare earth metal that is selected from Y, La, Ce, Pr, Nd, Pm, Sm, Eu, Gd, Tb, Dy, Ho, Er, Tm, Yb, Lu, distinct isotopes thereof, and mixtures thereof.

Embodiment 37. The microbead of embodiment 36, wherein the rare earth metal is selected from 89Y, 139La, 136Ce, 138Ce, 140Ce, 142Ce, 141Pr, 142Nd, 143Nd, 145Nd, 146Nd, 148Nd, 145Pm, 144Sm, 149Sm, 150Sm, 152Sm, 154Sm, 151Eu, 153Eu, 154Gd, 155Gd, 156Gd, 157Gd, 158Gd, 160Gd, 152Gd, 159Tb, 156Dy, 158Dy, 160Dy, 161Dy, 162Dy, 163Dy, 164Dy, 165Ho, 162Er, 164Er, 166Er, 167Er, 168Er, 170Er, 169Tm, 168Yb, 170Yb, 171Yb, 172Yb, 173Yb, 174Yb, 176Yb, 175Lu, or mixtures thereof.

Embodiment 38. The microbead of any one of embodiments 1 to 37, wherein the metal is distributed throughout the microbead.

Embodiment 39. The microbead of any one of embodiments 1 to 38, wherein the microbead has a glass transition temperature of about 60° C. or above 60° C., optionally about 70° C. or above 70° C., about 80° C. or above 80° C., about 90° C. or above 90° C., about 100° C. or above 100° C., about 115° C. or above 115° C., about 125° C. or above 125° C., or about 135° C. or above 135° C.

Embodiment 40. The microbead of any one of embodiments 1 to 39, wherein the microbead has a diameter of about 0.6 μm to about 20 μm, about 1 μm to about 15 μm, about 2 μm to about 10 μm, about 2 μm to about 6 μm.

Embodiment 41. The microbead of any one of embodiments 1 to 40, wherein the microbead is colloidally stable in water.

Embodiment 42. The microbead of any one of embodiments 1 to 41, wherein a surface of the microbead comprises functionalization for attachment to a biomolecule.

Embodiment 43. The microbead of embodiment 42, wherein the attachment is covalent attachment.

Embodiment 44. The microbead of embodiment 42 to 43, wherein the biomolecule is selected from a protein, an oligonucleotide, a small molecule, a lipid, a carbohydrate, or a mixture thereof.

Embodiment 45. The microbead of embodiment 42 to 43, wherein the biomolecule is an affinity reagent, optionally wherein the affinity reagent is an antibody.

Embodiment 46. The microbead of embodiment 45, wherein the antibody is specific for a cytokine, optionally a chemokine, an interferon, a lymphokine, a monokine, an interleukin, such as IL-1-36, tumor necrosis factor and colony stimulating factors.

Embodiment 47. The microbead of embodiment 44, wherein the antigen is a viral antigen.

Embodiment 48. The microbead of any one of embodiments 42 to 47, wherein the functionalization comprises a coating of silicon dioxide on the surface of the microbead, optionally the functionalization further comprises functionalizing the coating of silicon dioxide.

Embodiment 49. The microbead of embodiment 42, wherein the attachment to the biomolecule is non-covalent attachment.

Embodiment 50. The microbead of any one of embodiments 42 to 49, wherein the surface of the microbead is functionalized with avidin, streptavidin, neutravidin, or mixtures thereof.

Embodiment 51. The microbead of any one of embodiments 42 to 50, wherein the surface of the microbead is conjugated to the biomolecule.

Embodiment 52. The microbead of any one of embodiments 42 to 51, wherein the metal provides a barcode that identifies the biomolecule.

Embodiment 53. A population of microbeads as defined in any one of embodiments 1 to 52.

Embodiment 54. The population of microbeads of embodiment 53, wherein the population has a size distribution having a coefficient of variation (CV) of about 10% or less than 10%.

Embodiment 55. The population of microbeads of embodiment 54, wherein the coefficient of variation is of less than 5%.

Embodiment 56. The population of microbeads of any one of embodiments 53 to 55, wherein each microbead comprises a plurality of metals, the average amount across the population of microbeads of each metal of the plurality of metals is about 10% or within 10% of the average amount of another metal of the plurality of metals.

Embodiment 57. The population of microbeads of embodiment 56, wherein the plurality of metals comprises one or more enriched isotopes.

Embodiment 58. The population of microbeads of any one of embodiments 53 to 57, wherein the amount of each metal of one microbead of the population of microbeads is about 20% or within 20%, or about 10% or within 10%, or about 5% or within 5% of the amount of the same metal of another microbead of the population of microbeads.

Embodiment 59. The population of microbeads of any one of embodiments 53 to 57, wherein the amount of each metal of the population of microbeads has a distribution of a coefficient of variation of about 20% or less than 20%.

Embodiment 60. The population of microbeads of embodiment 59, wherein the amount of each metal of the population of microbeads has a distribution of a coefficient of variation of about 10% or less than 10%.

Embodiment 61. The population of microbeads of any one of embodiments 58 to 60, wherein the microbeads of the population of microbeads comprise the same metal in substantially the same amount.

Embodiment 62. The population of microbeads of embodiment 61, wherein the same metal is a plurality of metals and the microbeads comprise each metal of the plurality of metals in substantially the same amount.

Embodiment 63. A kit comprising a plurality of distinct populations of microbeads as defined in any one of embodiments 53 to 62.

Embodiment 64. The kit of embodiment 63, wherein each population of microbeads is distinguishable from another population of microbeads based on the metal or the plurality of metals of the microbeads.

Embodiment 65. The kit of embodiment 63 or 64, wherein the microbeads of at least one population of microbeads comprise a metal or a plurality of metals different from the metal or the plurality of metals of the microbeads of another population of microbeads.

Embodiment 66. The kit of embodiment 63 or 64, wherein the microbeads of at least one population of microbeads comprise a plurality of metals at a ratio different from the microbeads of another population of microbeads.

Embodiment 67. The kit of any one of embodiments 64 to 66, wherein the microbeads of each population of microbeads are conjugated to a different biomolecule.

Embodiment 68. Method of preparing a metal-encoded microbead comprising
polymerizing a structural monomer in the presence of a steric stabilizer in a nucleation stage to obtain a first mixture comprising polymerized structural monomer, unpolymerized structural monomer, and the steric stabilizer;
combining the first mixture with a metal-chelating monomer comprising a metal and a chelator attached to at least one polymerizable end group to obtain a second mixture,
wherein the chelator coordinates the metal at least at 3 sites and wherein the metal-chelating monomer is polymerizable with the structural monomer; and
polymerizing the second mixture to form a copolymer of the microbead;
wherein the structural monomer does not comprise the chelator.

Embodiment 69. The method of embodiment 68, wherein the metal is a plurality of metals.

Embodiment 70. The method of embodiment 68 or 69, wherein the structural monomer is polymerized in the nucleation stage to about 5% to about 20% completion based on the structural monomer.

Embodiment 71. The method of any one of embodiments 68 to 70, wherein the polymerizing of the second mixture occurs to about 75% to about 100% completion, about 80% to about 99% completion, about 85% to about 95% completion, about 85% to about 93% completion based on the structural monomer.

Embodiment 72. The method of any one of embodiments 68 to 71, wherein the structural monomer is as defined in embodiment 2 or 3.

Embodiment 73. The method of any one of embodiments 68 to 72, wherein the metal-chelating monomer is as defined in any one of embodiments 2, and 4 to 26.

Embodiment 74. The method of any one of embodiments 68 to 73, wherein the steric stabilizer is as defined in embodiment 27.

Embodiment 75. The method of any one of embodiments 68 to 74, wherein the metal is as defined in any one of embodiments 29 to 35

Embodiment 76. The method of any one of embodiments 68 to 75, the method further comprising functionalizing the microbead.

Embodiment 77. The method of embodiment 76, wherein the functionalizing of the microbead comprises mixing the polymerized second mixture with a third monomer to obtain a third mixture, the third monomer comprising a reactive functional group; and polymerizing the third mixture.

Embodiment 78. The method of embodiment 77, wherein the reactive functional group is selected from alcohol, aldehyde, carboxylic acid, epoxide, vinyl, alkyne, maleimide, or mixtures thereof.

Embodiment 79. The method of embodiment 76, wherein the functionalizing of the microbead comprises coating the microbead with silicon dioxide.

Embodiment 80. The method of embodiment 76, wherein the functionalizing of the microbead further comprises functionalizing the coating of silicon dioxide.

Embodiment 81. The method of any one of embodiments 68 to 80, the method further comprising conjugating the microbead to a biomolecule.

Embodiment 82. The method of embodiment 81, wherein the biomolecule is as defined in any one of embodiments 44 to 47.

Embodiment 83. The method of any one of embodiments 68 to 82, wherein the microbead has a diameter of about 0.6 μm to about 20 μm, about 1 μm to about 15 μm, about 2 μm to about 10 μm, about 2 μm to about 6 μm.

Embodiment 84. The method of any one of embodiments 68 to 83 wherein the microbead is as defined in any one of embodiments 1 to 52.

Embodiment 85. A microbead prepared by the method of any one of embodiments 68 to 83.

Embodiment 86. The microbead of any one of embodiments 1 to 52, wherein an interior structure of the microbead comprises the copolymer.

Embodiment 87. The microbead of any one of embodiments 1 to 52, and 86, wherein the metal-chelating monomer chelates a single metal atom and not a plurality of metal atoms.

Embodiment 88. The microbead of any one of embodiments 1 to 52, 86 and 87, wherein the microbead comprises a polymer seed that does not comprise the metal-chelating monomer.

Embodiment 89. The microbead of embodiment 88, wherein the polymer seed comprises a structural monomer; optionally wherein the structural monomer of the polymer seed is identical in structure to the structural monomer of the copolymer.

Embodiment 90. The method of embodiment 68, wherein an interior structure of the microbead comprises the copolymer.

Embodiment 91. A method of preparing metal-encoded microbeads, the method comprising:
providing an aqueous dispersion of swellable seed particles and an anionic surfactant;
contacting the aqueous dispersion with monomers comprising a structural monomer and a metal-chelating monomer, wherein the metal-chelating monomer comprises a metal and a chelator attached to at least one polymerizable end, wherein the chelator coordinates the metal at least at 3 sites, and wherein the structural monomer does not comprise the chelator;
allowing the monomers to diffuse into the seed particles to form an aqueous dispersion of swollen seed particles; and
initiating polymerization of the monomers in the aqueous dispersion of swollen seed particles.

Embodiment 92. The method of embodiment 91, wherein the structural monomer is selected from the group consisting of acrylic monomers, methacrylate monomers and vinyl monomers selected from the group consisting of styrene, divinylbenzene (DVB), ethyl vinyl benzene, vinyl pyridine, amino-styrene, methyl-styrene, dimethylstyrene, ethyl styrene, ethyl-methyl-styrene, p-chlorostyrene and 2,4-dichlorostyrene.

Embodiment 93. The method of embodiment 91 or 92, wherein the aqueous dispersion of swollen seed particles further comprises a steric stabilizer.

Embodiment 94. The method of embodiment 93, wherein the steric stabilizer is polyvinylpyrrolidone.

Embodiment 95. The method of any one of embodiments 91 to 94, wherein the providing the aqueous dispersion of swellable seed particles comprises preparing monodisperse swellable seed particles by emulsion polymerization.

Embodiment 96. The method of any one of embodiments 91 to 95, wherein the aqueous dispersion of swellable seed particles further comprises an organic compound with a molecular weight below 5000 Dalton and a water solubility at 25° C. of less than 10-2 g/L; and optionally an organic solvent in which said organic compound is soluble.

Embodiment 97. The method of any one of embodiments 91 to 96, wherein the swellable seed particles are monodisperse swellable seed oligomer particles.

Embodiment 98. The method of any one of embodiments 91 to 97, wherein the anionic surfactant is sodium dodecyl sulfate.

Embodiment 99. The method of any one of embodiments 91 to 98, wherein the structural monomer is as defined in embodiment 2.

Embodiment 100. The method of any one of embodiments 91 to 99, wherein the metal-chelating monomer is as defined in any one of embodiments 1, and 3 to 27.

Embodiment 101. The kit of embodiment 67, further comprising a reporter comprising a mass tag, wherein the reporter specifically binds a sample biomolecule specifically bound by at least one of the different biomolecules.

Embodiment 102. The kit of embodiment 101, wherein the sample biomolecule is an oligonucleotide, and wherein the at least one of the different biomolecules is an oligonucleotide that specifically hybridizes to the sample biomolecule.

Embodiment 103. The kit of embodiment 102, wherein the reporter comprises a plurality of oligonucleotides that hybridize to indirectly bind a plurality of mass tagged oligonucleotides to the sample biomolecule.

Embodiment 104. The kit of any one of embodiments 101 to 103, wherein the at least one of the different biomolecules is an affinity reagent such as an antibody.

Embodiment 105. The kit of embodiment 104, wherein the sample biomolecule is a viral particle, wherein the at least one of the different biomolecules is a first antibody that specifically binds the viral particle, and wherein the reporter comprises a second antibody that specifically binds the viral particle.

Embodiment 106. The kit of embodiment 104, wherein the sample biomolecule is a cytokine, wherein the at least one of the different biomolecules is a first antibody that specifically binds the cytokine, and wherein the reporter comprises a second antibody that specifically binds the cytokine.

Embodiment 107. The kit of embodiment 104, wherein the sample biomolecule is a cancer biomarker, wherein the at least one of the different biomolecules is a first antibody that specifically binds the cancer biomarker, and wherein the reporter comprises a second antibody that specifically binds the cancer biomarker.

Embodiment 108. The kit of any one of embodiments 101 to 107, wherein the at least one of the different biomolecules comprises a viral antigen, wherein the sample biomolecule is an antibody that specifically binds the viral antigen, and wherein the reporter comprises a secondary antibody that binds to the sample biomolecule.

Embodiment 109. The kit of any one of embodiments 101 to 108, further comprising a plurality of different reporters, wherein each of the different reporters binds a sample biomolecule specifically bound by a different biomolecule.

Embodiment 110. The kit of embodiment 109, wherein the plurality of different reporters each comprise the same mass tag.

Embodiment 111. The kit of any one of embodiments 101 to 110, wherein the mass tag comprises a metal nanoparticle.

Embodiment 112. The kit of any one of embodiments 101 to 110, wherein the mass tag comprises a metal chelating polymer.

Embodiment 113. The kit of embodiment 67, wherein at least one of the biomolecules is an enzyme substrate for a sample biomolecule.

Embodiment 114. The kit of embodiment 113, further comprising a reporter that specifically binds to the enzyme substrate when it has been modified by the sample biomolecule.

Embodiment 115. The kit of embodiment 113, further comprising a reporter that specifically binds to the enzyme substrate when it has not been modified by the sample biomolecule.

Embodiment 116. The kit of embodiment 115, wherein the enzyme substrate comprises a mass tag that is removed when the enzyme substrate is modified by the sample biomolecule.

Embodiment 117. The kit of any one of embodiments 101 to 116, wherein the microbeads from a plurality of different populations are in a first mixture of microbeads.

Embodiment 118. The kit of embodiment 117, further comprising a second mixture of microbeads comprising the same biomolecules as the first mixture of microbeads, wherein the microbeads of the first mixture comprise a sample barcode that is different from the microbeads of the second mixture.

Embodiment 119. The kit of embodiment 118, wherein the sample barcode is on the interior of the microbeads.

Embodiment 120. The kit of embodiment 119, wherein the sample barcode is a subset of metals chelated by the metal chelating monomer of the microbeads of the first and second mixture.

Embodiment 121. The kit of embodiment 118, wherein the sample barcode is on the surface of the microbeads of the first and second mixture.

Embodiment 122. The kit of embodiment 118, further comprising a plurality of sample barcodes in separate partitions that are functionalized to bind to the surface of microbeads from a plurality of different populations.

Embodiment 123. The kit of any one of embodiments 101 to 122, wherein the kit further comprises a panel of mass-tagged antibodies in admixture with one another, wherein at least some antibodies of the panel are specific for cell surface markers.

Embodiment 124. The kit of embodiment 123, wherein the plurality of antibodies is in admixture with microbeads of the kit.

Embodiment 125. The kit of embodiment 123 or 124, wherein at least some of the mass-tagged antibodies comprise metals identical to metals chelated by metal chelating monomers of microbeads of the kit.

Embodiment 126. The kit of any one of embodiments 101 to 125, further comprising a steric stabilizer in admixture with the microbeads of the kit.

Embodiment 127. The kit of embodiment 126, wherein the steric stabilizer is polyvinylpyrrolidone.

Embodiment 128. The kit of any one of embodiments 101 to 127, wherein the microbeads of the kit are in a solution buffered at a pH at or between 5 and 9.

Embodiment 129. The kit of any one of embodiments 101 to 125, wherein the microbeads are lyophilized.

Embodiment 130. The kit of any one of embodiments 101 to 125, wherein the microbeads are fused to a solid support.

Embodiment 131. The kit of embodiment 130, wherein the solid support is a microscope slide.

Embodiment 132. The kit of embodiment 130, wherein the solid support is an adhesive film.

Embodiment 133. The kit of any one of embodiments 101 to 132, further comprising one or more of a buffer, anticoagulant, fixation reagents, and permeabilization reagents.

Embodiment 134. A method comprising detecting the population of microbeads of any one of embodiments 53 to 62 by mass spectrometry.

Embodiment 135. The method of embodiment 134, further comprising calibrating a mass spectrometer used to detect the microbeads based on a mass spectrum obtained from the microbeads.

Embodiment 136. The method of embodiment 134, further comprising normalizing a mass spectrometry signal obtained from a plurality of mass tags based on a mass spectrum obtained from the microbeads.

Embodiment 137. A method of mass spectrometry analysis, comprising:
mixing the distinct populations of microbeads of any one of embodiment 53 to 62 with a sample, wherein microbeads of each population of microbeads are bound to a different biomolecule and wherein each population of microbeads is distinguishable from another population of microbeads based on the metal or the plurality of metals of the microbeads;
binding different sample biomolecules of the sample to the different biomolecules of distinct populations of microbeads;
binding, directly or indirectly, a reporter to each of the different sample biomolecules, wherein the reporter bound to each of the different sample biomolecules comprises a mass tag; and
detecting the metal and the mass tag of individual microbeads by mass spectrometry.

Embodiment 138. The method of embodiment 137, further comprising attaching the different biomolecules to the microbeads of the distinct populations of microbeads prior to the step of mixing the distinct populations of microbeads with the sample.

Embodiment 139. The method of embodiment 137 or 138, wherein the different sample biomolecules comprise oligonucleotides.

Embodiment 140. The method of any one of embodiments 137 to 139, wherein the different sample biomolecules comprise antibodies.

Embodiment 141. The method of any one of embodiments 137 to 140, wherein the different sample biomolecules comprise cytokines.

Embodiment 142. The method of any one of embodiments 137 to 141, wherein the different sample biomolecules comprise cancer biomarkers.

Embodiment 143. A method of mass spectrometry analysis using the kit of any one of embodiments 101 to 133 comprising:
mixing distinct populations of microbeads with a sample, wherein microbeads of each population of microbeads are bound to a different biomolecule and wherein each population of microbeads is distinguishable from another population of microbeads based on the metal or the plurality of metals of the microbeads;
binding different sample biomolecules of the sample to the different biomolecules of distinct populations of microbeads;
binding, directly or indirectly, a reporter to each of the different sample biomolecules, wherein the reporter bound to each of the different sample biomolecules comprises a mass tag;
and detecting the metal and the mass tag of individual microbeads by mass spectrometry.

Embodiment 144. A method of mass spectrometry analysis of a cellular sample using the microbeads of any one of embodiments 63 to 74 comprising:
providing a plurality of mass-tagged antibodies, wherein each antibody of the mass tagged antibody is conjugated to a polymer mass tag chelating multiple atoms of a metal or enriched isotope thereof;
contacting a sample with the plurality of mass-tagged antibodies;
detecting the mass-tagged antibodies bound to the sample and the microbeads by mass spectrometry.

Embodiment 145. The method of embodiment 144, further comprising calibrating the mass spectrometer used in the step of detecting, wherein the calibrating is based a mass spectrometry signal obtained from the microbeads.

Embodiment 146. The method of embodiment 144, further comprising normalizing a mass spectrometry signal obtained from the mass tags based on a mass spectrometry signal obtained from the microbeads.

Embodiment 147. The method of embodiment 144, further comprising quantifying the mass-tagged antibodies based on the average number of metal atoms for each of the mass-tagged antibodies and the detected mass spectrometry signal from the mass-tagged antibodies and the microbeads.

Embodiment 148. The method of embodiment 146 or 147, wherein the microbeads and the mass-tagged antibodies comprise the same metals.

Embodiment 149. The method of any one of embodiments 134 to 148, wherein the step of detecting comprises imaging mass cytometry.

Embodiment 150. The method of embodiment 149, further comprising quantifying or normalizing the mass-tagged antibodies at individual pixels or bound to individual cells based on a mass spectrometry signal detected from the microbeads.

Embodiment 151. The method of embodiment 149 or 151, wherein the microbeads are melted to a solid surface prior to the step of detecting.

Embodiment 152. The method of any one of embodiments 134 to 151, wherein the step of detecting comprises suspension mass cytometry.

Embodiment 153. The method of any one of embodiments 134 to 152, wherein the step of detecting comprises inductively coupled plasma mass spectrometry (ICP-MS).

Embodiment 154. The method of any one of embodiments 149 to 151, wherein the step of detecting comprises laser ablation ICP-MS or secondary ion mass spectrometry (SIMS).

Embodiment 155. The method of any one of embodiments 134 to 154, wherein the mass spectrometry is time-of-flight mass spectrometry (TOF-MS).

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

### Example 1

Polystyrene (PS) Microbeads with One or More Lanthanides Chelated by DTPA

Metal-encoded PS microbeads were synthesized by introducing polymerizable metal-DTPA complexes into dispersion polymerization reactions of styrene in ethanol as second stage aliquots. Synthesis of the components and microbeads and the materials used are described.

### Materials

Diethylenetriaminepentaacetic dianhydride (DTPA dianhydride, 98%), 2,2′-azobis(2-methylpropionitrile) (AIBN, 98%), polyvinylpyrrolidone (PVP, Mw ˜55 kDa), Triton-X305 (TX305, 70% solution in water), benzylamine (BA, 99%), allylamine (ALA, 99%), N-(3-aminopropyl) methacrylamide hydrochloride (APMAm, 98%), triethylamine (TEA, 99%), sodium acetate (anhydrous, ≥99%), ammonium acetate (≥98%), sodium carbonate (≥99%), hydrogen peroxide solution (H₂O₂, 30% in H₂O), and metal salts with the purity≥99.99% (trace metals basis), including yttrium (III) chloride hexahydrate (YCl₃·6H₂O), cerium (III) chloride heptahydrate (CeCl₃·7H₂O,), europium (III) chloride hexahydrate (EuCl₃·6H₂O), holmium (III) chloride hexahydrate (HoCl₃·6H₂O), lutetium (III) chloride hexahydrate (LuCl₃·6H₂O), and deuterium oxide (D₂O, 99.9%) were purchased from Sigma-Aldrich. 4-Vinylbenzylamine (VBA, ≥92%) was supplied by TCI America. Absolute ethanol (EtOH) was manufactured by Commercial Alcohol. Nitric acid (tracemetal grade, 68-69%), sulfuric acid (tracemetal grade), sodium hydroxide, and phosphate buffered saline (1×PBS solution, Fisher BioReagents) were purchased from Fisher Scientific. All of the above chemicals were used without further purification. Styrene (St, Sigma-Aldrich, ≥99%) was purified by passing through an alumina (Sigma-Aldrich, Activated, Neutral) filled column. Single-elemental standard solutions for inductively coupled plasma mass spectrometry (ICP-MS) calibration were purchased from PerkinElmer (Pure Plus). Seven-element-encoded microbeads for mass cytometry (MC) calibration and normalization, containing an average of ⁸⁹Y (69×10⁶), ¹¹⁵In (43×10⁶), ¹⁴⁰Ce (17×10⁶), ¹⁵¹Eu (10×10⁶), ¹⁵³Eu (11×10⁶), ¹⁶⁵Ho (5.8×10⁶), ¹⁷⁵Lu (7.5×10⁶), and ²⁰⁹Bi (5.8×10⁶) per bead, were as described in Liu et al. 2020.¹⁵ EQ™ Four Element (EQ4) Calibration Beads for Mass Cytometry (MC) was kindly provided by Fluidigm Canada. Deionized water was produced by a Millipore purification system with minimum resistivity of 18 MΩ·cm. Compressed nitrogen (99.998%, Praxair) was used as the protection atmosphere for polymerization reactions.

### Syntheses of Functional DTPA-Bis(Amide) Derivatives

In order to incorporate DTPA metal complexes into polystyrene (PS) microbeads, DTPA was first functionalized by reacting DTPA dianhydride with 4-vinyl-benzylamine, benzylamine, allylamine or aminopropyl methacrylamide (R—NH₂=VBA, BA, ALA or APMAm) in a 1:2 stoichiometric ratio. The synthesis method was developed from the protocols reported by Zhang et al.¹⁶ for DTPA-bis(vinyl benzylamide) (DTPA-VBAm₂), Aime et al.¹⁷ for DTPA-bis(benzylamide) (DTPA-BAm₂), and Shuhendler et al.¹⁸ for DTPA-bis(allylamide) (DTPA-ALAm₂) with some modifications.

In a typical experiment to prepare DTPA-VBAm₂, DTPA dianhydride (1 mmol) was mixed with 4-vinyl benzylamine (2 mmol) in anhydrous DMSO (5.0 mL) at room temperature and stirred overnight. NaOH (1M, 3 eqv) in ethanol was added to the reaction to form the trisodium salt of DTPA-VBAm₂, followed by dilution of the reaction mixture with 45 mL acetone to precipitate the DTPA salt. The precipitated DTPA salt was then collected by sedimentation and re-dissolved in ethanol. Three cycles of dissolution-precipitation-sedimentation were performed to purify the product. The product was dried under reduced pressure at room temperature overnight to remove the residual solvent and characterized by proton nuclear magnetic resonance (1H-NMR) using a Varian 500 MHz instrument (Agilent) in D₂O solution at room temperature.

### Syntheses of Metal Complexes of DTPA-Bis(Amide) Derivatives

Metal complexes of DTPA-bis(amide) derivatives (M (DTPA-R2)) used for bead synthesis were prepared by loading metal ions onto the DTPA-R2 in aqueous solutions. In a typical metal chelation experiment, 0.30 mmol DTPA-R2 trisodium salt and an equimolar amount of metal chloride (0.30 mmol) were separately dissolved in water (3 mL). The metal salt solution was then added to the DTPA derivative solution, while the pH was monitored and adjusted to 5.0˜6.0 with 0.01 M HCl and 0.01 M NaOH solution. During the chelation of Ce³⁺ ions to DTPA-VBAm₂, transparent solution of the mixture slowly became opaque with the addition of metal chloride solutions. In the experiment, 1˜2 mL ethanol was added to the mixture to keep the solution transparent. Each mixture was stirred for 3 h at room temperature. M (DTPA-R2) complexes were isolated by acetone precipitation and sedimentation. Note that unchelated metal ions are soluble in the acetone-water mixture, while the metal complexes of DTPA derivatives have low solubility in acetone. The precipitate was then dissolved in ethanol and precipitated with acetone for further purification. The final products of metal complexes were collected by sedimentation and dried under reduced pressure overnight at room temperature. ¹H-NMR was employed to characterize the metal complexes.

### Two-Stage Dispersion Polymerization

Two-stage dispersion polymerization (2-stage DisP) was used to prepare metal-encoded polystyrene (PS) microbeads in presence of polyvinylpyrrolidone (PVP) as a steric stabilizer and DTPA derivative metal complexes as metal ligands. After initiating the polymerization of styrene in ethanol in the presence of PVP, a warm solution of the desired amount of DTPA metal complexes in ethanol was introduced as the second stage aliquots at 2 h. The reaction was terminated 24 h after the initiation, with the styrene conversion above 90%. Table 1 describes a typical recipe of this 2-stage DisP used to prepare microbeads.

TABLE 1
A typical recipe for the two-stage dispersion
polymerization of styrene for bead synthesis

| Materials (g) | 1^{st} Stage ^{a} | 2^{nd} Stage ^{b} |
|---|---|---|
| Styrene | 6.25 | — |
| AIBN | 0.25 | — |
| PVP | 1.00 | — |
| TX305 | 0.35 | — |
| Ethanol | 18.75 | 15.00 |
| DTPA derivative-metal | — | c |
| complexes | | |

^{a} The reaction was initiated by immersing the flask into a 70° C. oil bath. Prior to the initiation, reaction solution was purged with nitrogen gas for 30 min.
^{b} Second stage aliquot was introduced to the reaction 2 h after the initiation.
c A desired amount of DTPA derivative-metal complexes (M(DTPA-R₂)) dissolved in ethanol was introduced to the aliquot. Details of the metal addition are described in Table 2.

### Example 2

Twelve batches of bead synthesis were prepared using the methods and materials of Example 1 and various amounts of the M (DTPA-R2) complex as the feed in the second stage. These complexes were modified with different functional groups and loaded with different types of metal ions as described in Table 2.

TABLE 2
DTPA derivative-metal complexes (M(DTPA-R₂)) feed in the
second stage aliquots of a series of 2-stage DisP.

| | | M (DTPA-R₂) Complex Feed (μmol) M | | | | |
|---|---|---|---|---|---|---|
| Samples | R | Y | Ce | Eu | Ho | Lu |
| Y-1 | VBAm ^{a} | 70.0 | | | | |
| Ce-1 | VBAm ^{a} | | 70.0 | | | |
| Ce-2 | BAm ^{b} | | 70.0 | | | |
| Ce-3 | ALAm ^{c} | | 70.0 | | | |
| Ce-4 | AmPMAm ^{d} | | 70.0 | | | |
| Eu-1 | VBAm ^{a} | | | 70.0 | | |
| Ho-1 | VBAm ^{a} | | | | 25.0 ^{e} | |
| Lu-1 | VBAm ^{a} | | | | | 25.0 ^{e} |
| 5E1 | VBAm ^{a} | 7.06 | 6.59 | 6.47 | 6.38 | 6.30 |
| 4E1 ^{f} | VBAm ^{a} | | 3.49 | 3.62 | 1.29 | 1.35 |
| 4E2 ^{f} | VBAm ^{a} | | 10.5 | 10.9 | 3.87 | 4.06 |
| 4E3 ^{f} | VBAm ^{a} | | 31.4 | 32.6 | 11.6 | 12.2 |

^{a} VBAm stands for vinylbenzylamide;
^{b} BAm stands for benzylamide;
^{c} ALAm stands for allyamide;
^{d} AmPMAm stands for amidopropyl methacrylamide
^{e} Smaller numbers of Ho and Lu complexes were added to these syntheses to avoid the saturation of MC detector by resulting microbeads.
^{f} The numbers of metal complex addition were designed to achieve microbeads generating similar levels of intensities among all five isotopes.

After terminating the reaction, the microbead dispersions were washed by sedimentation-redispersion cycles twice with absolute ethanol and four times with water to remove free stabilizers, unreacted monomers, and any smaller diameter particles. Dispersions of these washed microbeads were used for MC characterization, and aliquots were freeze dried to measure the solids content.

### Example 3

### Surface Modification and Secondary Antibody Attachment

Microbeads were coated with a silica shell and conjugated to a secondary antibody.

### Materials for Silica Coating and Bioconjugation

Tetraethyl orthosilicate (TEOS, 99%), (3-Aminopropyl) triethoxysilane (APTES, 99%), Succinic anhydride (99%), anhydrous dimethyl sulfoxide (DMSO, 99.9%) were purchased from Sigma-Alderich. Ammonia solution 25% (NH₄OH), MES buffer (0.5 M pH 5.5), phosphate buffered saline (1×PBS, pH 7.4), N-hydroxysuccinimide (NHS), N-(3-Dimethylaminopropyl)-N′-ethylcarbodiimide hydrochloride (EDC), NeutraAvidin (NAv), biotin-xx-goat anti-mouse (H+L lgG) and bovine serum albumin (BSA) were ordered from ThermoFisher Scientific. MaxPar® ¹⁷⁵Lu-labeled mouse anti-TNFα (human) (clone MAb11), cell staining buffer and cell acquisition solution were kindly provided by Fluidigm Canada.

Preparation of Metal-Encoded Microbeads Functionalized with Goat Anti-Mouse (Antibody) by Silica Coating.

Metal-encoded microbeads (Eu-1) were first coated with silica (SiO₂) by the Stöber process. Typically, an aliquot of bead dispersion containing 50 mg beads (solids) was washed by sedimentation-redispersion cycles with 5 mL ethanol-ammonia solution (ethanol:ammonia=99:1 vol.) in a centrifuge tube. TEOS (75 μL) was added to the bead dispersion to initiate the silica condensation reaction. The reaction was agitated on a sample rotator (40 rpm, ambient temperature) for 20 hours and quenched by four sedimentation-redispersion cycles using 98% vol. ethanol (5 mL). An aliquot of silica-coated Eu-1 bead (Eu-1/SiO₂) dispersion (1 mL, ca. 10 mg solids) was treated in a sonication bath for 5 min and further functionalized with amino groups (NH₂) by adding APTES (5 μL) to the Eu-1/SiO₂ bead dispersion. The NH₂ coating reaction was agitated in an oven at 40° C. for 20 hours and quenched by four cycles of sedimentation-redispersion washing with absolute ethanol (1 mL). To convert the functional groups on NH₂-modified microbead (Eu-1/NH₂) to carboxyl groups (COOH), an aliquot of freshly prepared Eu-1/NH₂ microbead dispersion (800 μL, ca. 8 mg solids) was washed with ethanol-TEA solution (1 mL, containing 0.2% vol. TEA) for four sedimentation-redispersion cycles, followed by addition of freshly prepared succinic anhydride solution in DMSO (100 μL, 10% wt. succinic anhydride). The COOH modification reaction was agitated at 40° C. overnight on a sample rotator (40 rpm) and then terminated by four cycles of sedimentation-redispersion washing with water (800 μL).

Next, NAv was conjugated to COOH-modified Eu-1 beads (Eu-1/COOH) by EDC/NHS coupling. In the conjugation reaction, an aliquot of Eu-1/COOH dispersion (100 μL, ca. 1% solids) was washed with MES buffer (100 μL, 0.1 M, pH 5.5) four times to exchange the solvent. The EDC/NHS activation reaction was initiated by adding the EDC/NHS solution (100 μL), containing EDC (8 mg) and NHS (12 mg) in MES buffer, to the washed Eu-1/COOH dispersion. After incubation for 15 min, the reaction solution was quickly switched to 1×PBS buffer (100 μL; pH 7.4) by 2 cycles of centrifugal washings and followed by the addition of NAV solution (50 μL, 2 mg/mL). The NAv conjugation reaction was incubated for 4 hours followed by removing the unattached NAv by 4 cycles of centrifugal washing with PBS buffer (100 μL).

Goat anti-mouse (GAM) secondary antibody was attached to Eu-1 microbeads by incubating NAv-modified Eu-1 beads (ca. 2 million beads) in a BSA-PBS solution (100 μL, 0.5% wt. BSA in PBS) containing biotin-xx-GAM (20 μg) for 2 hours. The excess GAM were then removed by four cycles of centrifugal washing with BSA-PBS solution (100 μL).

### Example 4

The size and size distribution and metal and metal distribution of the microbeads prepared using the methods and materials of Examples 1 and 2 were characterized.

### Characterization of Microbead Sizes and Size Distributions

Scanning electron microscope (SEM) images of microbead dispersion were collected to characterize the microbead diameters and diameter distributions, using a Hitachi S-5200 microscope. Typically, 2 μL of a diluted bead dispersion was dropped on a 300 mesh Formvar/carbon coated copper grid and allowed to dry. The diameters of microbeads were manually measured from multiple SEM images using ImageJ software. The mean average diameter, standard deviation (SD) and the coefficient of variation (CV, see Eq 2) were calculated based on at least 300 measurements.

$$CV = \frac{SD}{Mean} = \frac{1}{\overline{x}} \sqrt{\frac{{\sum }_{i = 1}^{n} {( {x}_{i} - \overline{x} )}^{2}}{n - 1}} \times 100 \% \qquad ( 2 )$$

### Acid Digestion of Microbeads

Microbead dispersions were digested with sulfuric acid and H₂O₂ prior to ICP-MS measurements. In a typical digestion experiment, sulfuric acid (500 μL) and microbead dispersion (100 μL, 0.5-2% solids content) were mixed in a 20 mL glass vial. The microbead dispersion in sulfuric acid was then heated to 250° C. on a hotplate and held for 40 min with magnetic stirring, followed with an addition of 30% H₂O₂ solution (50 μL). For ICP-MS analysis, the digestion solution was subsequently diluted with 2% HNO₃.

### Instrumentation

Inductively coupled plasma-mass spectrometry (ICP-MS). ICP-MS (iCAP-Q, Thermo Scientific) was used to quantify the metal ion concentration in the samples. Samples were analyzed in the kinetic energy discrimination (KED) measurement mode. The elemental standard solutions were sequentially diluted with 2% HNO₃ to a series of concentrations of 40, 20, 10, 1, and 0.1 ppb as the calibration solutions. Based on the calibration fitting curve, the metal content in each solution sample was determined. The detection limits for elements of interests were estimated to be below 10 ppt.

Mass cytometry (MC). The metal content of microbead was characterized bead-by-bead by a mass cytometry system (Helios® CyTOF, Fluidigm). A sample of seven-element-encoded microbeads (7E1), which was reported in Liu et al, 2020¹⁵, was employed as an internal standard and mixed with the diluted microbead dispersion samples. The mixture consisting of 7E1 reference beads and microbead samples was then introduced to the MC system at a speed of 30 μL/min, and the beads were individually but stochastically introduced into the ICP. After signal acquisition, singlet events were identified and gated on the dot-plot generated by MC. The means, medians, robust standard deviations (RSD) and robust coefficient of variations (RCV) of singlet signals were reported as unnormalized raw data. Robust statistics provide an alternative approach to classical statistical estimators such as mean, SD, and CV.

### Results

The styrene conversion of microbeads prepared using the methods of Examples 1-3 was above 90%.

As shown in Scheme 1 below, four different types of DTPA derivatives were prepared. This incorporation efficiency was tested using Ce(III)-DTPA complexes as described below.

### Syntheses of Functional DTPA-R₂ Derivatives

To covalently incorporate metal complexes of DTPA into PS microbeads, DTPA was modified with functional groups that can react with styrene during the DisP. In this design two of five carboxylates on DTPA were substituted with functional groups so the remaining three carboxylates in the functionalized DTPA derivatives could form charge-neutral complexes with lanthanide(III) ions, which promotes the binding stability and the ethanol solubility of their metal complexes in DisP.

The metal complexes were synthesized by the approach outlined in Scheme 1. DTPA dianhydride reacts effectively with primary amine to produce DTPA bisamides.^{21,22} Bis(amide) derivatives of DTPA are octadentate chelators that strongly bind with lanthanide ions. It was hypothesized that the binding between metal ions and DTPA-bisamides to be highly stable against metal dissociation and exchange during the bead synthesis, where different types of metal complexes of DTPA-bisamides are mixed in the reaction.

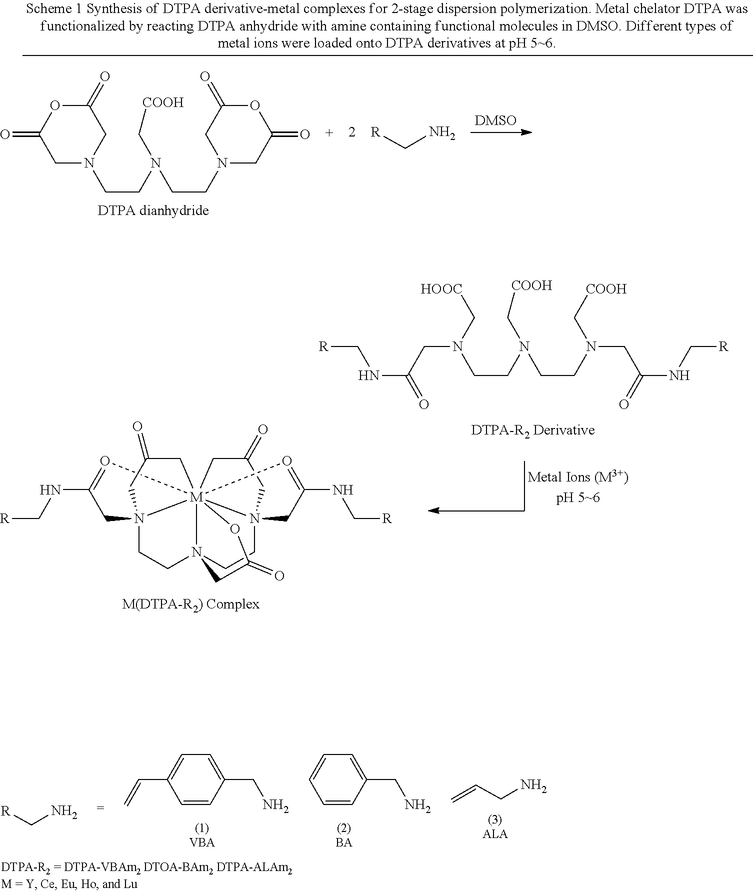

Two DTPA-bisamides were prepared that can react with styrene via chain transfer reactions via hydrogen abstraction from the benzylic carbon of the DPTA-bisbenzylamide (DTPA-BAm₂) or the allylic carbon^{25,26} of DTPA-bisallyamine (DTPA-ALAm₂). Results were compared with those obtained in the copolymerization with DPTA-bis-4-vinylbenzylamide (DTPA-VBAm₂), where the 4-vinylphenyl group would be expected to have a similar reactivity as styrene ²⁷⁻²⁹. The resulting products were characterized by ¹H-NMR to confirm their structures.

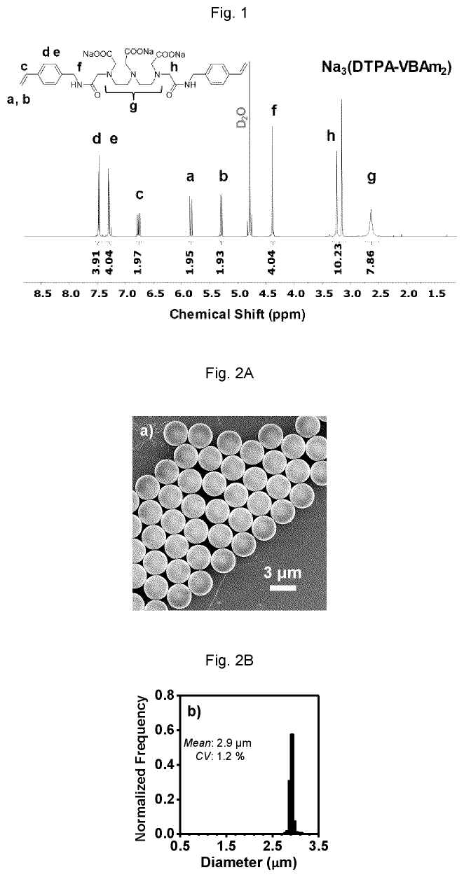

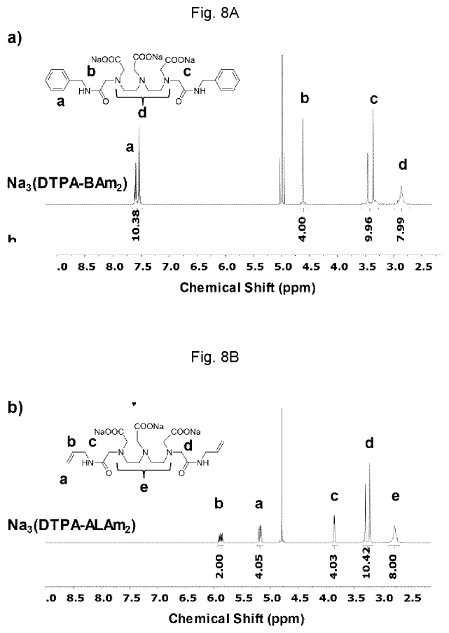

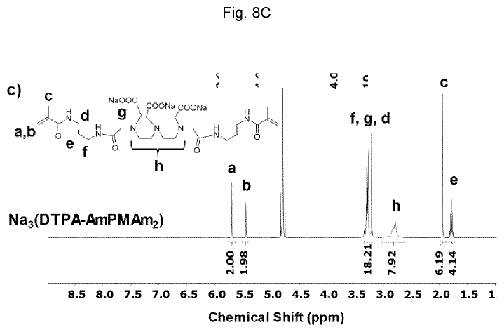

FIG. 1 presents the 1H-NMR spectrum of Na₃ (DTPA-VBAm₂), showing the distinct chemical shifts of protons in the vinyl groups at 5.3 (b, 2H), 5.8 (a, 2H), and 6.8 (c, 2H). The chemical shift at 4.4 ppm (f, 4H) indicates the formation of the amide linkage. The integration of protons from the vinyl, benzyl, and DTPA moieties confirms the bis-substitution of vinyl benzyl groups in each DTPA molecule. The ¹H-NMR spectra shown in FIGS. 8A and B match the spectra of DTPA-BAm₂ and DTPA-ALAm₂, respectively, reported in the literature ^{18,19,30} and verify the successful synthesis of these two DTPA-bis(amide) derivatives.

Testing Metal Incorporation into PS Microbeads with Ce (DTPA-R₂) Metal Complexes

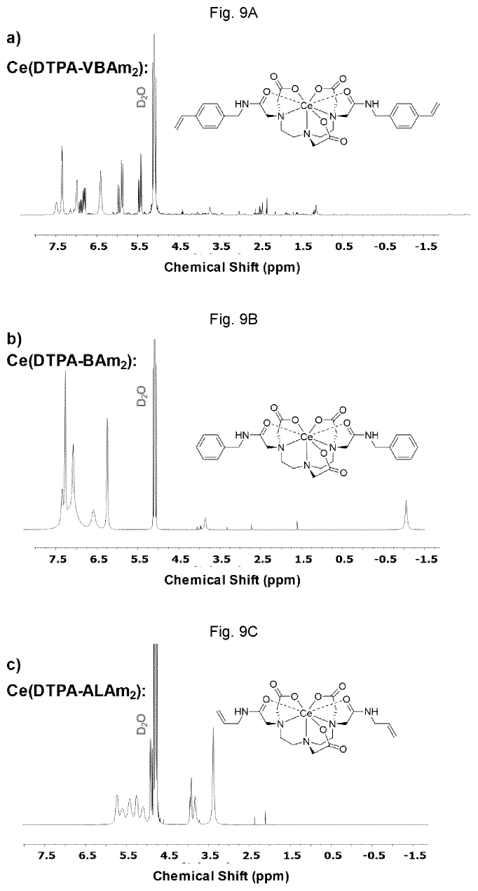

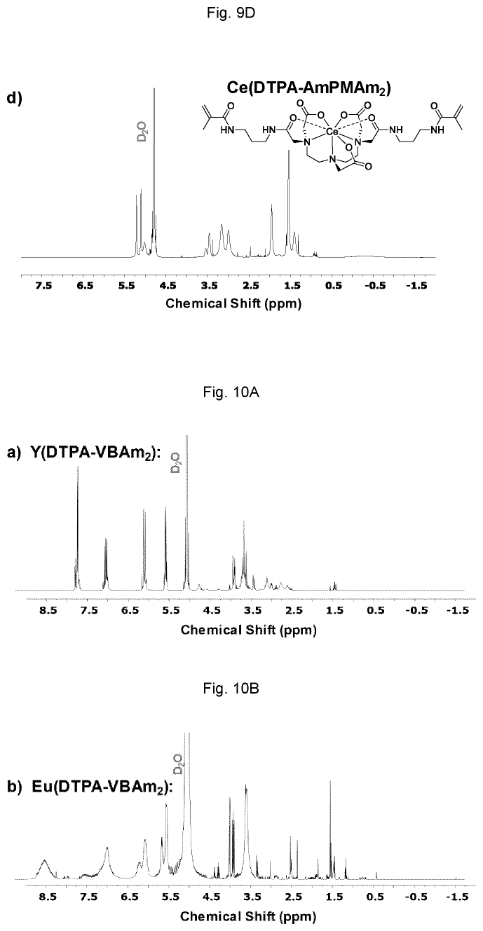

Syntheses of Ce (DTPA-R₂) Metal Complexes. In order to examine the incorporation activity of the different DTPA derivatives, Ce complexes of Ce (DTPA-VBAm₂), Ce (DTPA-BAm₂), and Ce (DTPA-ALAm₂) were prepared. Metal complexes were synthesized by adding an aqueous solution of CeCl₃ to DTPA-R₂ in water at pH 5.0˜6.0 in 1:1 molar ratio. The Ce (DTPA-R₂) complexes were precipitated by addition of acetone, and characterized by ¹H-NMR to verify the metal chelation. The 1H-NMR spectra of these Ce complexes are shown in FIG. 9A to C. Since Ce³⁺ is a paramagnetic NMR shift reagent, the chemical shifts of protons from these Ce complexes were shifted and broadened to some extent, indicating the chelating interactions of Ce³⁺. The crystal structures of several M (DTPA-BAm₂) complexes (M=In, Y and Lu) were reported by some researchers using X-ray diffraction to confirm the metal coordination in the complexes. ^{17,30} The metal coordination to (DTPA-R₂) chelators described herein may be similar to the reported M (DTPA-BAm₂) complexes due to the similarity of the material properties.

2-DisP with Ce (DTPA-R2) Metal Complexes. The incorporation efficiencies of the three Ce (DTPA-R2) complexes into PS microbeads were examined. As described in Table 2, equal molar amounts of Ce (DTPA-VBAm₂), Ce (DTPA-BAm₂) and Ce (DTPA-ALAm₂) were dissolved in ethanol and introduced to the Ce-1, Ce-2 and Ce-3 beads syntheses, respectively, as the second stage aliquots. From each of these syntheses, colloidally stable microbeads were obtained that retained their colloidal stability in water. The mean diameter (d) of Ce-1 microbeads was 2.9 μm with a narrow size distribution (CV=1.2%), as shown in FIG. 2. Ce-2 and Ce-3 microbeads were also uniform in size (CV<1.5%) but with slightly smaller mean diameters (d=2.1 and 2.5 μm, respectively), as summarized in Table 3, possibly due to the chain transfer effect of benzyl and allyl groups inhibiting the chain growth of PS.³¹

Metal ion incorporation in the reaction was determined by ICP-MS. The total metal ion content in the reaction was determined after digestion of the sample with H₂SO₄+H₂O₂. The microbeads in the reaction stock were separated by centrifugal sedimentation. The metal content in supernatant was also analyzed by ICP-MS to quantify the metal ions remaining in solution. As described in Liu et al, 2020¹⁵, the metal incorporation efficiency of bead synthesis was calculated based on the difference between unincorporated metal content in the supernatant and the total metal content in the reaction. This metal incorporation efficiency reflects the Ce complex incorporation into PS beads, since Ce is fed to the reactions as Ce-DTPA complexes and Ce ions in all three Ce-complexes are expected to remain chelated with DTPA derivatives during the bead synthesis due to their relatively high binding stability.²³

The Ce incorporation efficiency of three microbead syntheses are presented in Table 3. In Ce-1 synthesis, 74% of Ce (DTPA-VBAm₂) complex added to the reaction was incorporated into the PS beads. However, more than 97% Ce (DTPA-BAm₂) and Ce (DTPA-ALAm₂) remained in the solution after the reaction. In other words, only 2.7% Ce (DTPA-BAm₂) and 2.8% Ce (DTPA-ALAm₂) complexes added to the Ce-2 and Ce-3 syntheses were incorporated, respectively.

As a complementary measure of Ce content, Ce-1, Ce-2 and Ce-3 microbeads were examined by MC on a bead-by-bead basis, using calibration microbeads containing a known amount of ¹⁴⁰Ce. as a quantification standard. The Ce content in sample beads was evaluated by comparing the signal intensities between the sample beads and the calibration beads, assuming that the MC signal intensities from different microbeads were proportional to the metal content in the same measurement. 15 Table 3 summarizes the Ce content of these three batches of microbeads as determined by MC.

In MC, Ce-1 microbeads generated a sharp and strong ¹⁴⁰Ce signal (see FIG. 11) with a median intensity at 6000 counts per bead and an RCV of 6.2% (see FIG. 11A). The Ce content in Ce-1 microbeads was estimated to be 5.44×10⁷ Ce ions per bead (see Table 3). In contrast, Ce-2 and Ce-3 microbeads showed much weaker 140Ce signal intensities (FIGS. 11B and C respectively). The Ce content for the Ce-2 and Ce-3 microbead samples was essentially identical at 1.6×10⁶ and 1.6×10⁶ Ce ions/bead.

To confirm the incorporation mechanism of polymerizable Ce complex in the bead synthesis, another Ce complex, Ce (DTPA-AmPMAm₂) was prepared, employing methacrylamide as the functional group that can copolymerize with styrene. As in the preparation of Ce (DTPA-VBAm₂), the Ce (DTPA-AmPMAm₂) complex was synthesized by first reacting DTPA dianhydride with APMAm in DMSO and then chelating Ce³⁺ ions in water at pH 5˜6. The 1H-NMR spectra in FIG. 8(c) and FIG. 9(d) confirm the structure of Na₃ (DTPA-AmPMAm₂) and Ce (DTPA-AmPMAm₂), respectively. During the Ce chelation experiment, Ce (DTPA-AmPMAm₂) seemed more hydrophilic than Ce (DTPA-VBAm₂), because Ce (DTPA-AmPMAm₂) was fully soluble in the chelation solution without addition of ethanol. Ce-4 microbeads were prepared using an equal amount of Ce (DTPA-AmPMAm₂) complex as the ligand under a similar condition to Ce-1, Ce-2 and Ce-3 syntheses. As a result, Ce-4 microbeads also had a mean diameter of 2.9 μm with a narrow size distribution (CV=1.3%) and generated a bright ¹⁴⁰Ce signal with a median intensity at 4000 counts per bead and an RCV of 5.7% (see FIG. 11D).

Both Ce (DTPA-VBAm₂) and Ce (DTPA-AmPMAm₂) complexes were effectively incorporated into PS beads during DisP due to the reactive copolymerization between the vinyl benzyl amide/methacrylamide and styrene. The much less efficient incorporation of Ce (DTPA-BAm₂) and Ce (DTPA-ALAm₂) complexes is due to the low reactivity between benzyl amide/allyl amide and styrene as chain transfer agents under the bead synthesis conditions.

TABLE 3
The characteristics of four batches of Ce-encoded microbeads prepared by
2-stage DisP. of styrene with Ce complexes of different DTPA derivatives
(Ce(DTPA-VBAm₂), Ce(DTPA-BAm₂), Ce(DTPA-ALAm₂) and
Ce(DTPA-AmPMAm₂)) as the metal chelators.

| | | Bead Size ^{a} | | Ce Content ^{b} | | |
|---|---|---|---|---|---|---|
| | | d | | 10⁻⁶ × | | Incorporation |
| Sample | Chelator | μm | CV % | ions/bead | | Efficiency ^{c} % |
| Ce-1 | DTPA- | 2.9 | 1.2 | 54.4 | (±3.4) | 74 |
| | VBAm₂ | | | | | |
| Ce-2 | DTPA- | 2.1 | 1.3 | 1.6 | (±0.6) | 2.7 |
| | BAm₂ | | | | | |
| Ce-3 | DTPA- | 2.5 | 1.4 | 1.6 | (±0.6) | 2.8 |
| | ALAm₂ | | | | | |
| Ce-4 | DTPA- | 2.9 | 1.3 | 35.7 | (±3.5) | 51 |
| | AmPMAm₂ | | | | | |

^{a} The mean diameter (d) and Coefficient of Variation (CV) of the microbeads were characterized by SEM images of the microbead sample after sedimentation-redispersion washing;
^{b} The metal content per microbead were measured by MC using calibration beads as a standard; The mean and standard deviation of microbeads' metal content were evaluated based on the median and robust standard deviation of their MC signal intensities;
^{c} The incorporation efficiency of metal ions in bead synthesis was determined by comparing the total metal ion content in the reaction to the metal content remaining in the supernatant after sedimentation of the beads at the end of the reaction.

DTPA-VBAm₂ chelator was assessed for incorporation of other lanthanide metal ions into PS microbeads.

### Preparation of Metal-Encoded Microbeads Using M(DTPA-VBAm₂) Metal Complexes

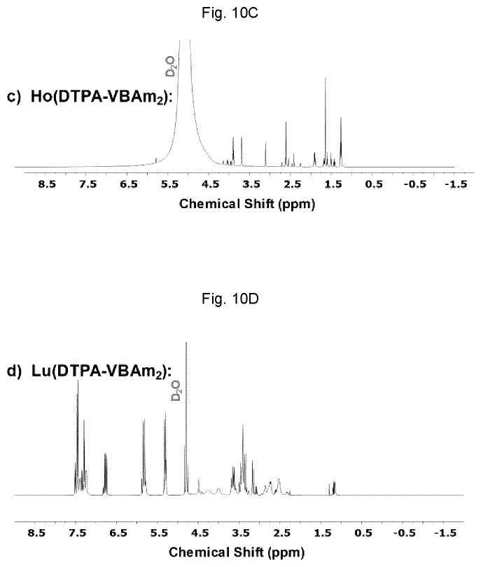

Complexes of DTPA-VBAm₂ with four other metal ions (Y³⁺, Eu³⁺, Ho³⁺ and Lu³⁺) were synthesized. These syntheses employed a methodology similar to that described above for Ce (DTPA-VBAm₂). The ¹H-NMR spectra of Y (DTPA-VBAm₂), Eu (DTPA-VBAm₂), Ho (DTPA-VBAm₂) and Lu (DTPA-VBAm₂) are presented in FIG. 10A to D respectively. Eu³⁺ and Ho³⁺ are paramagnetic NMR shift reagents. The chemical shifts in the NMR spectra of Eu (DTPA-VBAm₂) and Ho (DTPA-VBAm₂) present similar shift profiles to the spectrum of Ce (DTPA-VBAm₂). In contrast, Y³⁺ and Lu³⁺ are diamagnetic, the spectra of Y (DTPA-VBAm₂) and Lu (DTPA-VBAm₂) in FIGS. 10S and D show no shift effect. The chemical shifts of protons in vinyl benzyl functional groups (5-8 ppm) in these spectra resemble those of the chelator, whereas the chemical shifts of protons of DTPA in the aliphatic region (1-4 ppm) broaden, possibly due to the interconversion of different DTPA-VBAm₂ isomers associated with metal chelation^{30,32}. In the metal chelation experiments, it was noticed that Ho (DTPA-VBAm₂) and Lu (DTPA-VBAm₂) were less soluble in ethanol but more soluble in water than Ce (DTPA-VBAm₂). Without wishing to be bound by theory, this observation suggested that the polarity of Ho (DTPA-VBAm₂) and Lu (DTPA-VBAm₂) complexes are higher than that of Ce (DTPA-VBAm₂), possibly because of the stronger binding of Ho and Lu to the DTPA chelator²³. It can be appreciated that it is preferable that all the metal complexes are fully dissolved in the second stage aliquots during bead synthesis.

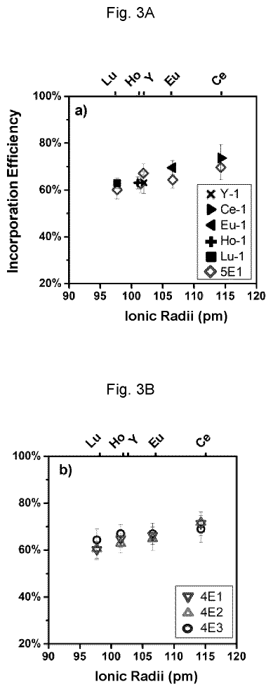

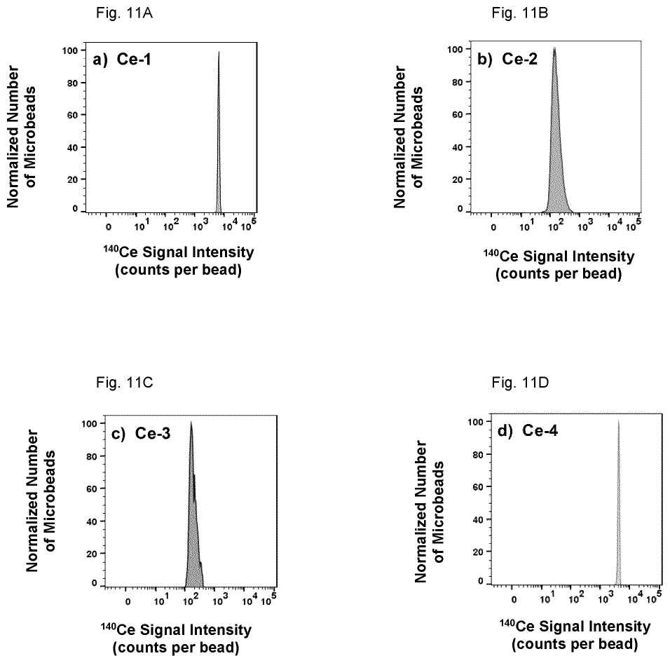

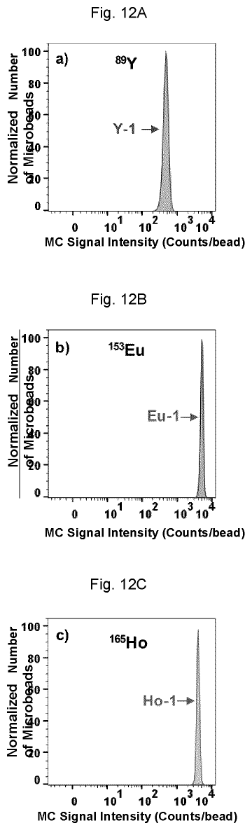

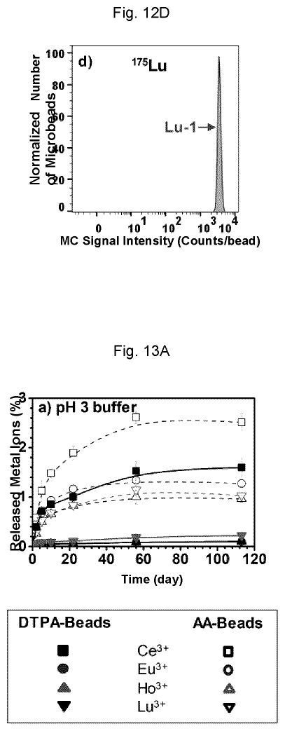

To examine the incorporation of these metal complexes in microbeads, each of these different M (DTPA-VBAm₂) complexes were used to prepare a series of microbeads (Y-1, Eu-1, Ho-1 and Lu-1) containing a single metal element (Y, Eu, Ho and Lu, respectively) in each synthesis, as described in Table 2. The microbeads obtained from Y-1, Eu-1, Ho-1 and Lu-1 syntheses were uniform and similar in size (see Table 4). The MC signal intensities of the encoded elements from these microbeads were strong and narrowly distributed, as presented in FIG. 12. The metal content evaluated by comparing the MC signal intensities from sample microbeads and the calibration beads in the same measurement confirmed that large numbers of elements were incorporated in each microbead, as summarized in Table 4. The incorporation efficiency of metals in these bead syntheses assessed as described above were all in the range of 65˜72% with a slight increase in this range for the lanthanide metal ions with the larger ionic radius. (See FIG. 3A).

A batch of five-element-encoded microbeads (5E1) was synthesized by dissolving 5 mg of each M (DTPA-VBAm₂) complex (M=Y, Ce, Eu, Ho and Lu) in 15 g ethanol as the second stage aliquot for the DisP reaction. In order to investigate the incorporation interference between different elements when they were mixed in one reaction, the incorporation efficiency of individual metals in the bead synthesis was characterized by ICP-MS.

The mean diameter of 5E1 microbeads was 2.7 μm with a narrow distribution (CV=1.2%). The incorporation efficiencies of these five metals shown in FIG. 3A indicates 63˜70% metal complexes introduced to the 5E1 reaction were incorporated into the beads. Different metal ions added to the 5E1 bead reaction were incorporated into microbeads with similar efficiency. The MC signal intensities of isotopes encoded in 5E1 beads were measured together with seven-element-encoded microbeads¹⁵ as a calibration standard. FIG. 4A summarizes the median MC signal intensities of 5E1 beads with error bars representing the RSD of the signal intensities. The signal intensities of ¹⁴⁰Ce, ^{151, 153}Eu, ¹⁶⁵Ho and ¹⁷⁵Lu were in the optimal MC sensitivity range (>300 counts per bead) with narrow signal distributions (RCV<10%). The signal intensity of ⁸⁹Y from 5E1 beads was less than 100 counts per beads, due to the low transmission coefficient of the 89 amu channel in MC.¹⁴ Using the MC signal intensities from the calibration beads, the metal content per 5E1 bead was calculated. The results are presented in FIG. 4B. The content of Ce, Eu, Ho and Lu in 5E1 beads were in a similar range, 3.4˜4.0×10⁶ ions per bead. The Y content is slightly higher (5.7×10⁶ ions/bead) as a consequence of there being more Y atoms in 5 mg Y (DTPA-VBAm₂) than in 5 mg of the other Ln (DTPA-VBAm₂) samples.

### Control of Metal-Ion Incorporation in Microbead Synthesis

FIG. 3A summaries the metal incorporation efficiency results in the batches of bead syntheses (Y-1, Ce-1, Eu-1, Ho-1, Lu-1 and 5E1) described in Table 2 using M (DTPA-VBAm₂) metal complexes as the ligands. The x-axis in FIG. 3 represents the ionic radius of the incorporated metal ions. The incorporation efficiency of different metals in these batches of beads overlapped in a very close range of 62˜74%, even though the metal feed in these bead syntheses varied. It appears that the reactivity of the 4-vinylbenzylamide group is independent of the metal ion bound to the chelator. As a result, the metal incorporation efficiencies in the bead syntheses were consistent when M (DTPA-VBAm₂) metal complexes were employed as the ligands.

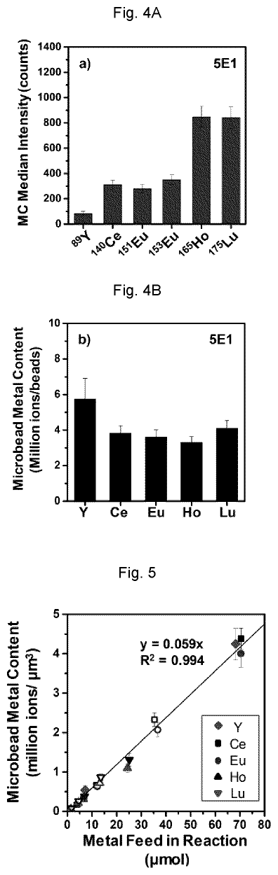

The relationship between the feed of M (DTPA-VBAm₂) complexes and the metal content of the resulting PS microbeads was investigated. In FIG. 5, the metal content found in the microbeads is plotted against the amount of metal complex fed to the synthesis in solid symbols. Taking into account of the slight batch-to-batch variation in microbead sizes, the metal content in each microbead was divided by the volume of the beads as the metal concentration in microbeads shown as the y-axis in FIG. 5. The solid data points in FIG. 5 show a linear dependence of the metal content in the microbeads on the feed of metal complexes in the bead synthesis. The linear regression model fit of these data serves as a convenient guide to design the level of metal content in metal-encoded microbeads by varying the amount of M (DTPA-VBAm₂) introduced in each reaction. This situation is a striking improvement in experimental design and execution compared with the use of metal salts+acrylic acid as a means of preparing metal-containing PS microbeads. With this new methodology, a library of metal-encoded microbeads with various levels of metal content can be prepared that can be individually identified as classifier beads for MC bead-based bioassays.

Preparation of a Set of Metal-Encoded Classifier Microbeads with Distinct Levels of Metal Content

To prepare a library of classifier beads to the maximum variability, one has to encode multiple metal isotopes into microbeads with finely controlled metal content so that the microbeads with various metal content can be individually resolved in MC. A set of reaction conditions is provided that enables the synthesis of classifier beads containing three distinct levels of metal ions with baseline separation as described in Example 2. Intensity levels that differed by factors of three were selected. However, other intensity levels can be selected. For example, the intensity levels that differ by factors of 2, or 2,5 can be selected.

Y (DTPA-VBAm₂) (70 μmol) takes longer to dissolve in ethanol (15 g) possibly due to its high polarity. Accordingly, ions other than Y ions were used in these reactions. However, it can be understood that Y can be a suitable metal for the microbeads of the present disclosure. For example, Y can be used with a different metal-chelating monomer. For example, Y can be used at a different concentration. Three samples of four-element PS microbeads containing Ce, Eu, Ho, and Lu were synthesized at concentration levels that differed by a factor of three. These samples are denoted as 4E1, 4E2 and 4E3 as shown in Table 2. The aim was to obtain microbeads that produce MC signal intensity levels of ¹⁴⁰Ce, ¹⁵¹Eu, ¹⁵³Eu, ¹⁶⁵Ho and ¹⁷⁵Lu that are at 0.2, 0.6 and 1.8 times, respectively, to the MC intensity levels of 7-element-encoded microbeads. 15

The microbeads obtained from the syntheses of 4E1, 4E2 and 4E3 were colloidally stable and free of coagulum in the reaction stock. The average diameters of these microbeads after washing by sedimentation-redispersion were in the range of 2.8˜3.0 μm with narrow size distributions (CV<1.5%), as shown in Table 4. As presented in FIG. 3B, the incorporation efficiencies of different metal ions in these three bead syntheses were consistent and close to the levels observed in the syntheses described in FIG. 3A.

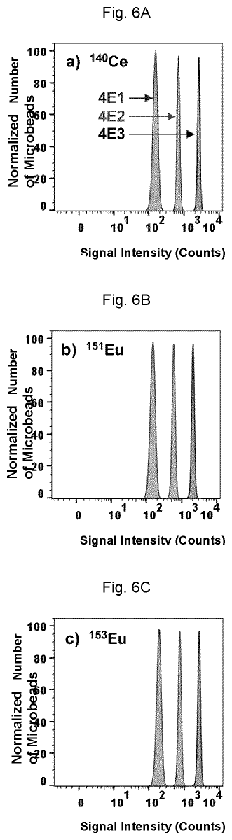

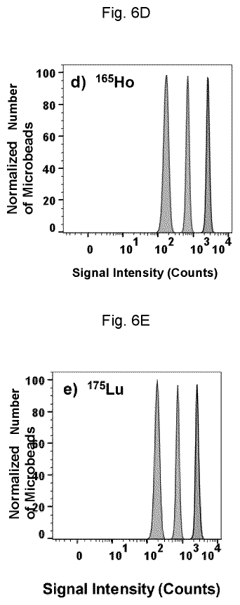

The MC signal intensities of five encoded lanthanide isotopes from these three batches of microbeads are presented in FIG. 6 with the panels sorted by element. The average MC signal intensities of ¹⁴⁰Ce, ¹⁵¹Eu, ¹⁵³Eu, ¹⁶⁵Ho and ¹⁷⁵Lu from 4E3 beads were the highest among these three batches of beads and at similar levels, that were 2560, 2053, 2590, 2540 and 2530 counts per bead, respectively. The signal intensity distributions of 4E3 beads were narrow with the RCV values less than 9%, indicating the narrow distributions of encoded lanthanide elements in the beads. 4E1 and 4E2 beads also generated sharp and narrowly distributed MC signals with intensities at around 200 and 700 counts per beads, respectively, for all the encoded isotopes. The histograms in FIG. 6 show clear baseline resolution among these three batches of microbeads. The signal intensities of these three batches of beads increased ca. three times from 4E1 to 4E3 beads.

By comparing the MC signal intensities with those of the 7-element calibration beads, the metal content in these three microbeads was evaluated. The values were plotted against the metal feed in the bead synthesis as open symbols in FIG. 5. These open symbol data points followed the linear trend line observed from previous bead syntheses presented in FIG. 5.

The synthesis of these three microbeads serves as an example demonstrating the convenience of preparing a library of beads encoded with five types of lanthanide isotopes at three different concentration levels using M (DTPA-VBAm₂) as copolymerizable metal complexes in the bead synthesis. According to Eq. 1, the maximum variability of this bead library shown in FIG. 6 is 1023.

Compared with the method previously used in Liu 2020 for the synthesis of the 7-element bead, the incorporation efficiency across different metals has been shown to be more consistent. In Liu 2020, the incorporation efficiency varied drastically across different metals, making controlled labelling of the microbeads difficult. For example, in Liu 2020, Lu³⁺ has the highest incorporation among all seven elements, approximately 50%, while Ce³⁺ had the lowest efficiency. Only about 15% of the Ce³⁺ added to the reaction mixture was incorporated into the beads. The incorporation efficiency of the lanthanides generally showed a trend that smaller ions had a higher incorporation efficiency.

As shown herein, metal complexes of the structure M (DTPA-VBAm₂) copolymerize effectively with styrene in two-stage dispersion polymerization reactions in ethanol in the presence of polyvinylpyrollidone. This reaction leads to PS microbeads with diameters on the order of 2 μm and very narrow size distributions (CV≈1-2%). Metal complex incorporation efficiency is on the order of 60 to 70%, slightly increasing in this range for the lanthanide metal ions with the larger ionic radius. This efficiency appears to be independent of the amount of metal complex introduced into the reaction for the range of concentrations examined. This feature of the reaction allows one to dial in a particular metal content for a sample of PS microbeads. This is a useful strategy for preparing both calibration beads for MC and for preparing classifier beads for bead-based MC assays as described for example in Example 6. Eu-labeled beads with goat-anti-mouse antibody bound to its surface were effective in detecting a Lu-labeled mouse IgG.

### Example 5

Metal Leakage from Microbeads

The stability of microbeads prepared according to Examples 1 and 2 with respect to the leaching of metal ions to aqueous medium was assessed under several experimental conditions. The sample 4E3 beads prepared by 2-stage DisP with M (DTPA-VBAm₂) was used for this test. Samples of washed microbeads (0.5% solids) were suspended in 30 mL of three different buffers that also contained 1% PVP: sodium acetate (50 mM, pH 3.0), ammonium acetate (10 mM, pH 7.0), sodium carbonate (200 mM, pH 10.5). The samples were stored at 4° C. At various time intervals, each sample was first vortexed and then 2 mL aliquots were taken. These aliquots were centrifuged and the supernatant was collected to measure the metal content by ICP-MS. The percentage of metal that had leached from the beads was determined by ICP-MS based on the metal content in the supernatant in comparison with the metal content in the bead dispersion. In parallel, a batch of microbeads prepared by the 2-stage DisP AA approach was also subjected to the same experiment conditions.

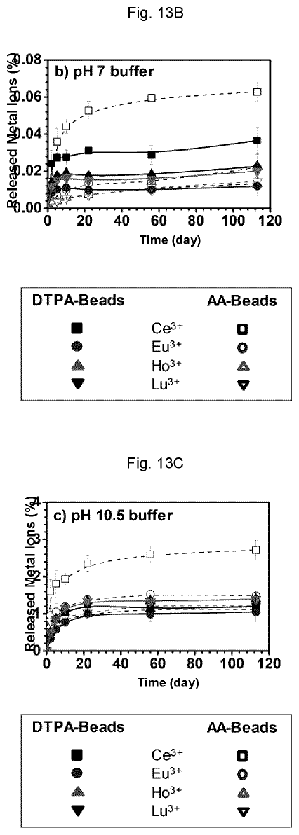

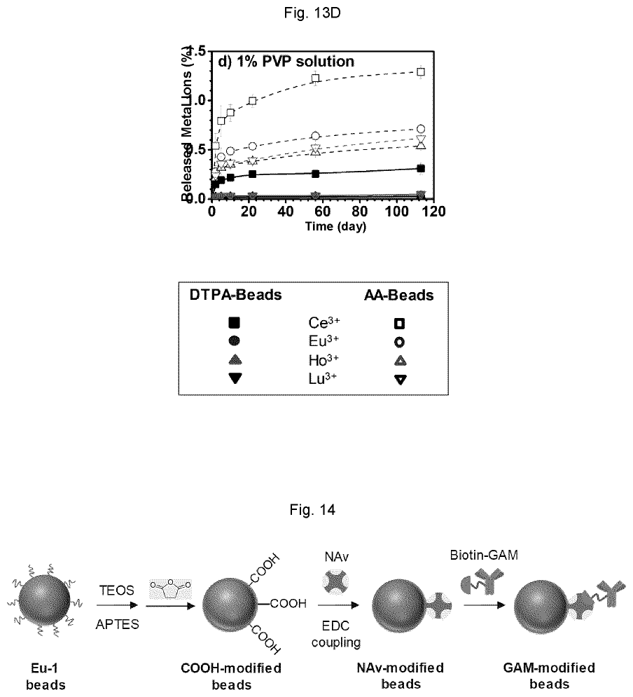

The stability of a batch of M (DTPA-VBAm₂)-encoded beads (4E3) against metal leaching during storage and under typical application conditions was tested. These DTPA-beads were stable against loss of metal ions (<1.5%) upon storage in buffers of pH values ranging from 3 to 10 as described in FIG. 13

### Leaching Stability of Incorporated Metals

Metal-encoded microbeads used in MC have to be stable against metal ions leaching during storage and under typical application conditions. Samples of these microbeads at 0.5 wt % were dispersed in three aqueous buffers (sodium acetate, pH 3.0; ammonium acetate, pH 7.0; sodium carbonate, pH 10.5) and stored at 4° C. In parallel, another set of microbead samples were tested in the same buffers, but these beads were prepared by 2-stage DisP using acrylic acid (AA) as the ligands to incorporate metal ions, as reported in Abdelrahman et al., 2009.9 The leakage of each element from 4E3 beads was monitored into these solutions as a function of time by ICP-MS.

Metal leaching from M (DTPA-VBAm₂)-encoded microbeads stored in unbuffered water in the presence of 1 wt % PVP was also monitored.

FIG. 13 presents the results of metal leaching experiments, where the solid symbols refer to DTPA-beads (4E3) and open symbols refer to the AA-beads as a comparison. Very minimum detectable leakage (<0.06%) of any incorporated element was observed from both microbeads in the pH 7 buffer over more than 100 days as described in FIG. 13B. For the 4E3 DTPA-beads stored in PVP solution at 4° C., very little release of the metal elements (up to 0.3%) was detected over 100 days, while the AA-beads lost slightly more metal ions (ca. 1.3% for Ce³⁺) upon storage under the same condition. Microbeads aged in acidic (pH 3) and basic (pH 10.5) buffers showed higher levels of ion loss. Here the leakage of elemental ions reached plateau values in 20 days, with no further loss over the next 80 days. The loss of Eu³⁺, Ho³⁺ and Lu³⁺ from DTPA-beads were small (<0.2%) in pH 3 buffer, whereas it was higher for Ce³⁺ (ca. 1.5%) presumably due to the weaker binding stability between Ce³⁺ and DTPA chelator. Overall, DTPA-beads show less metal ion leakage and stronger stability under four tested conditions.

TABLE 4
The metal content in microbeads prepared by 2-stage
DisP of styrene with M(DTPA-VBAm₂) metal complexes

| | Bead Size ^{a} | | Metal Content ^{b} | | | | |
|---|---|---|---|---|---|---|---|
| | d | CV | Y | Ce | Eu | Ho | Lu |
| Samples | μm | % | 1 × 10⁶ ions/bead | | | | |
| Y-1* | 2.4 | 1.5 | 30.4(16%) | — | — | — | — |
| Ce-1* | 2.9 | 1.2 | — | 54.4(6.2%) | — | — | — |
| Eu-1* | 2.8 | 1.4 | — | — | 46.0(8.6%) | — | — |
| Ho-1* | 3.1 | 1.2 | — | — | — | 15.5(9.4%) | — |
| Lu-1* | 2.9 | 2.0 | — | — | — | — | 16.7(10%) |
| 5E1* | 2.7 | 1.3 | 5.75(20%) | 3.83(11%) | 3.67(11%) | 3.31(9.7%) | 4.10(11%) |
| 4E1* | 2.8 | 1.0 | — | 2.15(15%) | 2.29(15%) | 0.64(16%) | 1.00(16%) |
| 4E2* | 2.9 | 1.4 | — | 9.54(8.6%) | 8.70(10%) | 2.57(10%) | 3.71(10%) |
| 4E3* | 3.0 | 1.4 | — | 36.9(7.3%) | 31.5(8.6%) | 9.95(9.0%) | 13.7(8.9%) |

^{a} The mean diameter (d) and Coefficient of Variation (CV) of the microbeads were characterized by SEM images of the microbead sample after sedimentation-redispersion washing;
^{b} The metal content per microbead were measured by MC using calibration beads as a standard.

### Example 6

### Classifier Beads

### Surface Modification and Antibody Attachment

M (DTPA-VBAm₂)-encoded beads functionalized with a goat anti-mouse (GAM) secondary antibody (Ab) as described in Example 3 were examined for the specific binding between GAM-modified Eu-1 microbeads using a ¹⁷⁵Lu-labeled Ab reporter by MC.

### Test of Specific Binding Between Classifier Beads and Reporters by Mass Cytometry

For the specific binding experiment, the GAM-modified microbeads (Eu-1/GAM) as the classifier were first dispersed in the BSA blocking solution (50 μL, 3% BSA in PBS). After 30 min's incubation, a cell staining solution (50 μL), containing ¹⁷⁵Lu-labeled mouse IgG (6.25 μg) as the reporter, was introduced to the Eu-1/GAM dispersion. The dispersion of classifier and reporter was incubated for 2 hours followed by two cycles of centrifugal washing with cell acquisition solution (100 μL) to remove unbounded reporters. The bindings of reporter on classifier beads were examined by MC. To test the non-specific binding, NAv-modified Eu-1 beads (without the attachment of GAM) were employed as a negative control to the same binding experiment condition and examined by MC as well.

FIG. 14 illustrates the strategy used to surface functionalize the microbead sample and subsequently attach Abs to the microbead surface. As described in Example 3, the microbeads were first coated with a thin silica shell followed by introduction of amino groups in a two-step silica sol-gel reaction as reported by Abdelrahman³³. TEOS and APTES were employed in the silica coating process. NH₂-modified Eu-1 microbeads were obtained with silica shell ca. 10 nm thick and surface amino groups. The amino groups on microbead surface were then converted to carboxyl groups (COOH) by reacting with succinic anhydride in DMSO. The carboxylate groups serve as functionality for attaching bioaffinity agents and help reduce non-specific binding of reporters to the microbead surface. Neutravidin (NAv) was covalently conjugated to the COOH groups on the microbead surface by EDC/NHS coupling. Finally, biotinylated GAM was attached to the NAv-modified microbead surface through the strong biotin-avidin affinity.

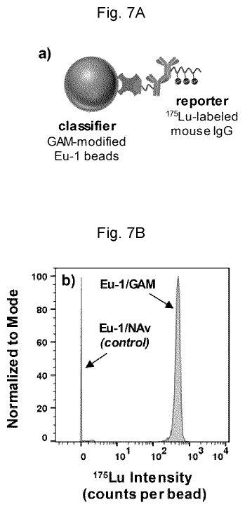

To validate the functionality of GAM-modified Eu-1 microbeads as a classifier, ¹⁷⁵Lu-labeled mouse IgG was chosen as a reporter and incubated with Eu-1/GAM beads. As a secondary Ab, GAM has a specific high reactivity towards the mouse IgG in the reporter as indicated in FIG. 7(a). To test the antigen recognition, a suspension of the GAM-modified microbeads (Eu-1/GAM) as were first dispersed in a blocking solution containing bovine serum albumin (50 μL, 3% BSA in PBS). After a 30 min incubation, a cell staining solution (50 μL), containing ¹⁷⁵Lu-labeled mouse IgG (6.25 μg) as the reporter, was introduced and incubated for 2 h at 23° C., followed by two cycles of centrifugal washing with cell acquisition solution (100 μL) to remove unbounded reporters. The sample was examined by MC. As shown in FIG. 7B, a sharp 175Lu signal peak on Eu-1/GAM microbeads with a median intensity at 460 counts per bead (RCV=15%) indicates strong specific bindings between the GAM-modified microbeads and the reporter. In contrast, the signal obtained for a sample of Eu-1/NAV microbeads without GAM attached gave a signal that was statistically minimized to zero.

### Example 7

Encoding Microbeads with CE/DTPA Using a Methacrylamide Derivative Ce (DTPA-AmPMAm₂))

To prepare a methacrylamide-functionalized DTPA chelator, N-(3-aminopropyl)-methacrylamide hydrochloride (APMAm) (2 mmol) was dissolved in anhydrous DMSO (5.0 mL), followed with the addition of triethylamine (TEA, 5.5 mmol) to deprotonate the amine hydrochloride. DTPA dianhydride (1 mmol) was then introduced to the APMAm-DMSO solution and the reaction was stirred overnight at room temperature. NaOH (1M, 3 eqv) in ethanol was added to the reaction to form the trisodium salt of DTPA-AmPMAm₂, followed by filtration with a syringe filter (0.2 μm). The filtrate was diluted with 45 mL acetone to precipitate the product. The precipitated DTPA salt was then collected by sedimentation and dissolved in ethanol. Three cycles of dissolution-precipitation-sedimentation were performed to purify the product. The product was dried under reduced pressure at room temperature overnight to remove the residual solvent. 1H-NMR was employed to confirm the structure of the product.

To prepare Ce complex of DTPA-AmPMAm₂, DTPA-AmPMAm₂ (0.3 mmol) and CeCl₃ 7H₂O (0.3 mmol) were dissolved separately in 5 mL and 1 mL of DI H₂O, respectively. The Ce solution was then added to the DTPA-AmPMAm₂ solution, while the pH was monitored and adjusted to 6.0 with 0.01 M NaOH solution. The mixture was stirred for 3 h at room temperature. The Ce (DTPA-AmPMAm₂) complex was isolated by precipitation with acetone and sedimentation. The precipitate was then dissolved in ethanol and precipitated a second time with acetone for further purification. The final product was also characterized by 1H-NMR.

A typical two-stage dispersion polymerization was employed to prepare polystyrene (PS) microbeads encoded with Ce (DTPA-AmPMAm₂) complex. PVP-55 (1.00 g), AIBN (0.25 g), Triton X305 (0.35 g) were added to a flask and fully dissolved in a mixture of styrene (6.25 g) and ethanol (18.75 g). The solution was sealed in the flask and stirred with an overhead mixer. After 30 min nitrogen purging at room temperature, the polymerization reaction was initiated by immersing the flask in an oil bath at 70° C. As the second stage aliquot, a warm solution of the Ce (DTPA-AmPMAm₂) complex (54 mg, ca. 70 μmol) in ethanol (15.0 g) was injected into the reaction 2 h after the initiation of the reaction. The reaction was terminated 24 h after the initiation. A stable coagulum-free microbeads dispersion was obtained and stored at 4° C.

The particles obtained in this example were coagulum free with a narrow size distribution (CV=1.3%) and a mean diameter of 2.9 μm. The Ce content in microbeads measured by mass cytometry was ca. 2.7×10⁷ ions per bead. Assessed by ICP-MS, 49% Ce added in the reaction remained in the solution after reaction termination. Therefore, the incorporation efficiency for Ce (DTPA-AmPMAm₂) complex in microbeads was estimated to be 51%.

Similar methods can be used for other Ln ions.

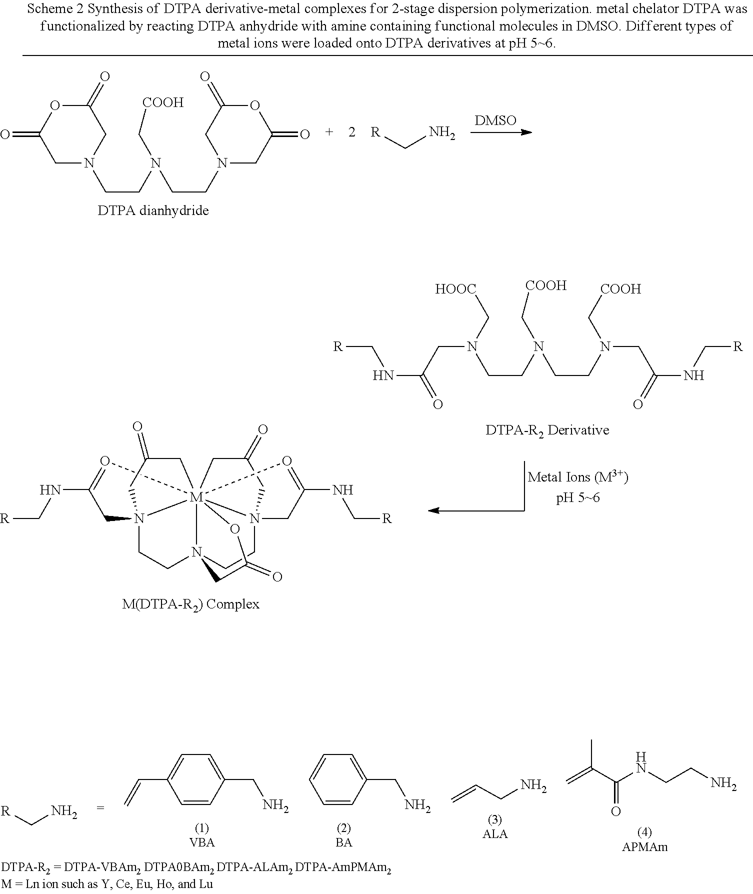

### Example 8

### Bioconjugation of Microbeads-Nucleic Acid

It can be appreciated that nucleic acid or oligonucleotide can be conjugated to the microbead using methods available in the art. For example, the 3′-hydroxyl group can be conjugated to carboxylic acid (such as succinic acid) functional group on a functionalized microbead. For example, a phosphoramidite derivative of a nucleotide can be used for the conjugation. Oligonucleotides may be functionalized for conjugation (e.g., to a microbead or biomolecule described herein) may easily be obtained commercially, such as from Integrated DNA Technologies (IDT). For example, oligonucleotides functionalized with biotin (e.g., a destionbiotin), amine, an alkyne modifier, a thiol modifier, Acrydite, or an NHS ester such as azide, may be conjugated as described further herein.

### Example 9 Multiplexed Bead Based Assay by MC

### Experimental

### Materials

The synthesis and characterization of metal-encoded microbeads employed in this example are presented in Example 10. Buffers used in this example were purchased from Life Technologies. Antibodies (Abs), biotinylated Abs, and cytokine standards were purchased from Biolegend and R&D Systems. (see Table 5) Streptavidin conjugated gold nanoparticles (AuNP, 10 nm, 10 OD) were bought from Abcam. Frozen human peripheral blood mononuclear cells (PBMC) were purchased from Immunospot (CTL, LP-188 HHU20130715). Nanogold®-Streptavidin (Nanoprobe), EQ™Four Element Calibration Beads (EQ4) and Cell-ID™ Pd Barcoding Kits were kindly provided by Fluidigm Canada.

TABLE 5
Material information: cytokine standards, capture Abs,
and detection biotinylated Abs used in this research

| Cytokine Analyte Standard | | | | | |
|---|---|---|---|---|---|
| Recombinant Human | | Capture Ab | | Detection Biotinylated-Ab | |
| Protein (carrier-free) | Catalog # | Clone # | Catalog # | Clone # | Catalog # |
| IL-18/IL-1F4 | B001-5 ^{a} | 125-2H | D044-3 ^{a} | 159-12B | D045-6 ^{a} |
| TNFα | 570102 ^{b} | MAb1 | 502801 ^{b} | MAb11 | 502903 ^{b} |
| IL-6 | 715104 ^{b} | MQ2-13A5 | 501101 ^{b} | MQ2-39C3 | 501201 ^{b} |
| IFNγ | 570202 ^{b} | MD-1 | 507501 ^{b} | 4S.B3 | 502503 ^{b} |
| IL-4 | 574002 ^{b} | 8D4-8 | 500701 ^{b} | MP4-25D2 | 500803 ^{b} |
| CD163 | 1607-CD ^{a} | 215930 | MAB16071 ^{a} | 215901 | BAM16072 ^{a} |
| CXCL-9/MIG | 392-MG ^{a} | 49106 | MAB392 ^{a} | Goat (poly) | BAF392 ^{a} |
| IL-10 | 571002 ^{b} | JES3-9D7 | 501401 ^{b} | JES3-12G8 | 501502 ^{b} |
| IL-1β | 579402 ^{b} | JK1B-1 | 508201 ^{b} | JK1B-2 | 508301 ^{b} |

^{a} Vendor: R&D systems.
^{b} Vendor: BioLegend.

### Preparation of a Set of Metal-Encoded Classifier Beads

To develop bead-based assays in MC, a library of microbeads labeled with various metal ions were prepared that can be individually identified by MC. The method of encoding microbeads with controlled levels of metal ions by introducing polymerizable metal complexes of the structure M (DTPA-VBAm₂) in two-stage dispersion polymerization (DisP) reactions is described herein. The experimental details of the preparation and treatment of the metal-encoded classifier beads are found in Example 10. A panel of 11 binary-metal encoded 3 μm polystyrene (PS) microbeads were prepared employing methods of the present disclosure as described in Example 10.

To prepare classifier beads that can capture the target analytes in immunoassays, each type of the metal-encoded beads was modified with one type of Ab specific for a cytokine analyte.

### Stimulation of PBMC for Cytokine Secretion

A frozen commercial human PBMC sample (CTL Immunospot) was thawed and added to a pre-warmed RPMI serum-free media (10 mL) containing 0.5 mL of CTL anti-aggregate wash supplement (20×). The PBMCs in the sample were spun down by centrifugation (8 min, 300 rpm). The supernatant was aspirated after centrifugation. The PBMCs were then resuspended with a warm serum-containing complete RPMI (10 mL). An aliquot of this PBMC suspension in RPMI (4.8 mL, 4.8×10⁶ cells) was transferred to a PS tube and stimulated with a PBS solution (100 μL) containing phorbol myristate acetate (PMA, 25 nmol) and ionomycin (100 nmol). The sample with stimulants was incubated for 5 h at 37° C. with 5% CO₂. The simulated PBMC suspension was then centrifuged to settle the cells. The supernatant was collected as the stimulated sample and stored at −80° C. prior to bead-based assays. Another aliquot of the PBMC suspension in RPMI media was treated under the same conditions in parallel except for the addition of stimulants (PBS only). The unstimulated sample was collected as a control in this experiment.

### Bead-Based Sandwich Immunoassays for Mass Cytometry

To optimize the assay condition, several multiplexed bead-based assays were carried out using a series of solutions with known concentrations of mixtures of cytokines and chemokines as standard samples. These mixtures were analyzed by MC to plot standard curves. FIG. 16 shows the assay procedure developed in this study.

A series of four-plex assays were carried out to analyze IL-4, IL-6, TNFα and IFNγ in 10 standard samples. In these experiments, four types of capture Ab-coated metal-encoded classifier microbeads were first mixed in approximately equal numbers in BSA solution (0.5% BSA in PBS). The classifier bead dispersion was then transferred to 10 wells of a 96-well filter plate (filter cut-off: 0.45 μm). Each well containing ca. 2 million beads in the dispersion (50 μL) can analyze one standard sample. A series of 10 standard solutions (50 μL each) consisting of four analytes at 0, 0.31, 1.22, 4.88, 19.5, 78.1, 313, 1250, 5000, 20000 μg/mL were added to each well, respectively. The mixture of standard solution and classifier beads in each well was first agitated with a pipette and then incubated on a microplate shaker (1200 rpm for 30 s, then 900 rpm for 2 h) at room temperature. After the incubation, the solution in the mixture was removed by reduced pressure filtration through the build-in filter at the bottom of the well. The classifier beads with analytes captured on their surface were washed by two redispersion-filtration cycles with washing buffer (200 μL, 0.025% TWEEN® 20 in PBS) to remove possible uncaptured analytes. Once the washing buffer was removed from the well by filtration, a detection Ab cocktail (100 μL, 0.5% BSA in PBS) containing four types of biotinylated Abs (2.5 μg/mL for each Ab) was transferred to each well. The classifier beads were redispersed with detection Abs by pipette agitation. The mixture of classifier beads and detection Abs was incubated at room temperature for 1 h on a microplate shaker (900 rpm). After incubation, the mixture was filtered and washed by two redispersion-filtration cycles with washing buffer to remove unbound detection Abs on the classifier beads. The classifier beads on the filter were then redispersed with a dispersion (100 μL) of streptavidin-conjugated gold nanoparticles (AuNPs) as the reporter. The reporter dispersion was prepared by diluting the AuNP dispersion from the vendor (e.g. 200-times) with 0.5% BSA buffer. The mixture of classifier beads and AuNP reporter was incubated on a shaker (900 rpm) for 1 h and washed by two filtration-redispersion cycles with washing solution and two cycles with PBS buffer (100 μL). In order to measure multiple assays in a single MC run, the assay sample (100 μL) in each well was stained with a unique palladium barcoding solution (40 μL, 3× dilution of a stock solution in Cell-ID™ Pd Barcoding Kit). The barcoding staining reaction was incubated for 30 min with agitation and quenched by two filtration-redispersion cycles with 0.5% BSA solution (200 μL) and two cycles with water (100 μL). A total of 10 barcoded assay samples were combined in one test tube and examined by MC employing EQ4 beads as a calibration standard.

In a typical multiplex assay of a sample with unknown analyte concentrations, different types of metal-encoded classifier beads that can capture the target analytes were first mixed in equal numbers in BSA solution and then transferred to a 96-well filter plate. In each well, the classifier bead dispersion (50 μL) can analyze one unknown sample by adding an aliquot (50 μL) of the sample solution to the well. The assays were then incubated, washed, stained with detection Abs and AuNP reporter in a similar procedure as that described in the above assays of standard samples.

### Instrumentation

Mass cytometry (MC). The microbead samples were characterized bead-by-bead by mass cytometry (Helios™ a CyTOF® system, Fluidigm). In a typical MC measurement, barcoded immunoassay samples and EQ4 beads as an internal standard were pooled into a test tube and introduced to the MC system at a speed of 30 L/min. After signal acquisition, the MC signals were normalized using signals from the EQ4 beads and debarcoded to separate the results from the different assays. To analyze the MC result of one assay sample, the singlets of all the classifier beads included in the assay were first identified and gated on the ¹⁴⁰Ce-¹⁴²Ce dot-plot. Each type of classier beads was then gated based on their signature signals of metal encoding. The median signal intensities of the reporter, e.g. ¹⁹⁷Au for AuNPs are reported as the results.

### Results

Multiplexed bead-based immunoassays in MC were developed. Metal-encoded microbeads were employed as the solid support for an immunoassay and gold (Au) NPs as the reporter. The capture Ab of each immunoassay was coupled to one of the microbead sets, each of which was comprised of microbeads with a uniform, distinct content of heavy metal isotopes. Once coupled with capture Abs, microbeads from different sets as classifiers could be pooled together for the multiplexed assays and separated later after data acquisition. Data were acquired on a MC and analyzed by FlowJo™ software. (see FIG. 16 for the experimental design) The immunoassay resulted in a variable ¹⁹⁷ Au signal intensity of the Au NP reporter, proportional to the amount of analyte bound to the surface of each microbead. Since MC quantifies the content of different heavy metal isotopes in each microbead, the pool of microbeads could be separated into individual bead set with the median ¹⁹⁷Au signal intensity of NP reporter for each bead set. Due to this feature, many assays could be carried out simultaneously, allowing for the multiplexed quantification of multiple analytes in a single measurement. In addition, the concentration of the analytes in the sample could be determined by extrapolation from an internal standard.

### Syntheses of Metal-Encoded Classifier Beads by Dispersion Polymerization

The synthesis was envisioned for 32 types (2⁵=32) of classifier microbeads binary encoded by varying the incorporation of five types of lanthanide metal ions (La³⁺, Pr³⁺, Tb³⁺, Ho³⁺, and Tm³⁺) with intensity levels of either 0 or ca. 1000 (±20%) counts per bead. In addition, Ce³⁺ ions can be incorporated into each of the beads at a similar level. The cerium isotopes ¹⁴⁰Ce and ¹⁴²Ce can serve as a microbead identifier for the convenience of gating classifier signals in the MC results. As a demonstration, a set of 11 binary-metal encoded 3 μm polystyrene (PS) microbeads for bead-based assays were synthesized by a series of two-stage dispersion polymerization (DisP) following the bead synthesis protocol described herein in Example 4.

The bead synthesis and characterization are described in Example 10. A scanning electron microscopy image of bead sample C-1 (see Table 6) is presented in panel (a) of FIG. 22. It shows that these microbeads were uniform with a mean diameter of 3.0 μm and a CV of 1%. The median intensity levels of MC signals of the encoded isotopes were in the range of 1000˜1200 counts per bead. Table provides a summary of the beads prepared, their mean diameters (and CV values) as well as the labeling pattern and signal intensities in MC detection. These 11 types of microbeads were uniform (CV˜1%) and shared a similar size with mean diameters in the range of 2.8 to 3.0 μm. For antibody attachment, the beads were coated with antibodies, and the Abs attached to the respective classifier beads are listed in Table.

TABLE 6
Summary of the particle size, median intensities of MC signals,
and target analytes of classifier microbeads prepared by two-stage
DisP of styrene with M(DTPA-VBAm₂) metal complexes.

| | Bead Size ^{a} | | MC Median Signal Intensities ^{b} | | | | | | |
|---|---|---|---|---|---|---|---|---|---|
| Microbead | d | | (counts per bead) | | | | | | Target |
| ID | (μm) | CV | ¹³⁹La | ¹⁴⁰Ce | ¹⁴¹Pr | ¹⁵⁹Tb | ¹⁶⁵Ho | ¹⁶⁹Tm | Analytes ^{c} |
| C-1 | 3.0 | 1% | 1060 | 1100 | 1120 | 1130 | 1180 | 1110 | IL-18 |
| C-2 | 2.8 | 1% | 862 | 881 | — | — | — | — | TNFα |
| C-3 | 2.9 | 1% | — | 857 | 871 | — | — | — | IL-6 |
| C-4 | 3.0 | 1% | — | 1050 | — | 1130 | — | — | IFNγ |
| C-5 | 2.9 | 1% | — | 791 | — | — | 979 | — | IL-4 |
| C-6 | 2.9 | 1% | — | 874 | — | — | — | 984 | CD163 |
| C-7 | 2.9 | 1% | 900 | 937 | — | 1170 | — | — | CXCL-9 |
| C-8 | 2.9 | 1% | — | 815 | 864 | 960 | — | — | IL-10 |
| C-9 | 2.8 | 1% | — | 844 | | 935 | 958 | — | IL-1β |
| C-10 | 2.8 | 1% | — | 778 | — | 963 | — | 947 | * |
| C-11 | 2.8 | 1% | — | 768 | — | — | 898 | 898 | * |

^{a} The mean diameter (d) and the coefficient of variation (CV) of microbead samples were evaluated by measuring the diameters of at least 300 microbeads in their SEM images;
^{b} The median signal intensities of microbeads were measured by MC using EQ4 beads as a calibration standard. The robust coefficient of variation (RCV) of the signal intensities for each type of these microbeads was in the range of 7~9% (no shown in the table);
^{c} To capture the target analytes in assay samples, capture Abs were covalently coupled to the surface of microbeads
* No target analyte was assigned to the microbeads in this study. The microbeads can be surface modified with bioaffinity reagents for the capture of analytes.

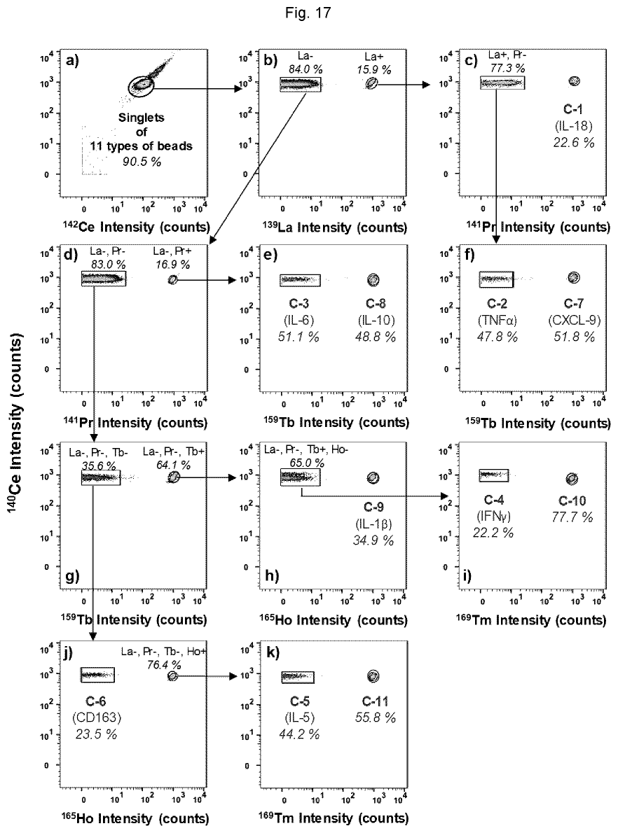

In addition, the metal ions incorporated in these microbeads generated MC signals at a similar intensity level of 800˜1200 counts per bead. Since all the microbeads (C1 to C11) carried Ce³⁺ that produced Ce signals at a similar level in MC, a mixture of all the beads were analyzed in a single MC measurement and isolated the singlets of 11 types of microbeads in one gating from the ¹⁴⁰Ce-¹⁴²Ce dot-plot. [see FIG. 17(a)] The dot-plots in FIG. 17(b-k) demonstrate the gating strategy to individually identify each type of classifier microbeads based on their signature signals of metal encodings. Because the microbeads in this set of classifier beads were uniform with MC signal intensities at similar levels, a gating template was created in FlowJo software based on this microbead gating strategy to simplify the data analysis process of multiplex assays.

### Initial Optimization of Bead-Based Sandwich Immunoassay Conditions

To test and optimize the reagents and conditions used in multiplexed sandwich immunoassays, these immunoassays were used to measure the cytokine levels in a series of solutions with known concentrations of cytokines as standard solutions. The effects of different assay reagents and conditions on the assay results were evaluated by examining the standard curves from these measurements. In the first stage of the assay development, a series of four-plex assays of standard solutions was used to test the candidate reporters, which were commercially available streptavidin-conjugated nano-sized Au reporters, and to optimize the reporter concentration in the assays. The assays were then expended to nine-plex assays to investigate the effect of detection Ab concentration on the assay performance.

In an exemplary experiment, a series of solutions was prepared containing known concentrations of target cytokines by diluting the standard solutions with 0.5% BSA-buffer (0.5% BSA in PBS). Each of these standard solutions (50 μL) was mixed with a cocktail of Ab-modified classifier beads (50 μL) in a filter plate. During the incubation, the Ab-modified classifier beads in the assay were allowed to capture their target analytes in the sample. After 1 h incubation, these classifier beads were washed by filtration to remove the unbound molecules in the sample. A cocktail of biotinylated detection Abs (100 μL) was then added to the assay followed by redispersing the classifier beads. In this step, the biotinylated Abs in the cocktail can recognize the analyte molecules captured on the classifier bead surface. These classifier beads in the assay were then washed on the filter to eliminate unbound detection Abs. Next, a streptavidin-conjugated Au mass tag dispersion (100 μL) was applied to the assay as a reporter. These mass tags were able to attach to the biotinylated detection Abs on the classifier beads by the streptavidin-biotin interaction.⁴⁷ After washing off the unattached reporter particles, the assay sample was examined by MC for the metal content in individual microbead event. The exemplary design of this bead-based sandwich immunoassay is illustrated in FIG. 16.

After signal acquisition, the assay results were analyzed in FlowJo software through their dot-plot diagram. FIG. 17 demonstrates the gating strategy employed in the study. The singlet events of all classifier beads were first isolated in the panel (a) of FIG. 17. Each classifier beads in the gated singlet events was then individually identified through a series of gating steps in the dot-plot diagrams shown in the panel (b-k) of FIG. 17. The ¹⁹⁷Au signal in each classifier event was then examined and reported as the assay signal.

Selection of Reporter. To find a proper metal-labeled mass tag to report the analyte binding on classifier beads, two types of commercially available streptavidin-conjugated reporters were examined, small diameter gold clusters (NanoGold®, d=1.4 nm) and somewhat larger diameter gold nanoparticles [AuNP, d=10 nm). These were employed as reporters in a series of four-plex assays analyzing standard solutions consisting of four analytes at 12 concentrations.

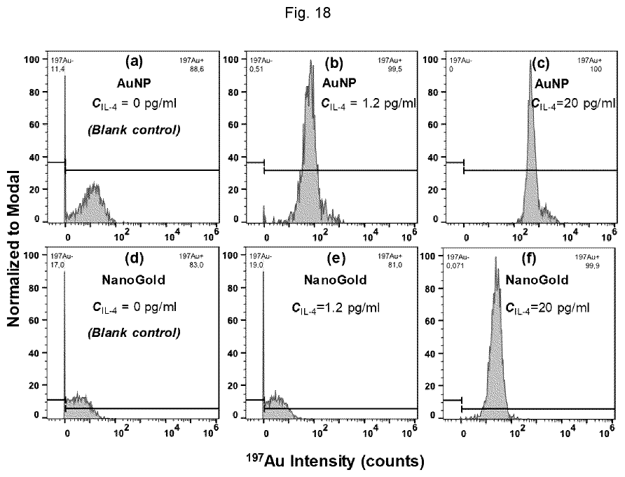

The histograms shown in FIG. 18 demonstrate the signal intensities of reporters on IL-4-classifier beads in the four-plex assays. AuNP was employed as the reporter in the assays shown in FIG. 18(a-c). A few positive ¹⁹⁷Au signals were recorded in FIG. 18(a) when IL-4 was absent in the blank control solution. The median value of all the events in this plot was reported as a background noise control to reflect the non-specific binding of reporter NPs to the classifier beads. The intensity peaks of ¹⁹⁷Au signals on the IL-4-classifier beads were shifted upfield in the panel (b) and (c) of FIG. 18. as the IL-4 concentration in the assays increased from 1.2 to 20 μg/mL. At 1.2 pg/mL, some events with the ¹⁹⁷Au signal intensity at nearly 0 count per bead were recorded in FIG. 18(b). These “0” events could be instrument noise due to artifacts during the signal acquisition process or due to some classifier beads carrying small numbers of reporters that were below the MC detection limit. To eliminate the uncertainties and simplify the data analysis process, the median intensity values of the positive events (reporter signal intensity>1 count per bead) in the positive samples were reported and plotted in this study.

In the histograms shown in panels (d), (e), and (f) of FIG. 18, NanoGold® was employed as the reporter to analyze standard solutions containing IL-4 at concentrations of 0, 1.2, and 20 μg/mL, respectively. Much weaker intensities of the NanoGold® reporter signals for ¹⁹⁷ Au on the IL-4-classifier beads were observed, compared with the results of assays using AuNPs as the reporter. More events with signal intensities of ˜0 were present in the histograms shown in panel (e) compared with panel (b) of FIG. 18.

A typical log-log standard curve of a bead-based assay starts with a low and flat region when the analyte concentration is low. The curve then rises with the increase of the analyte concentration, followed with a plateau at a higher concentration.^{41, 43, 48} In this Example, the detection range of an assay was estimated based on the slope region of the standard curve. Within this slope region, analyte concentration is generally measurable.

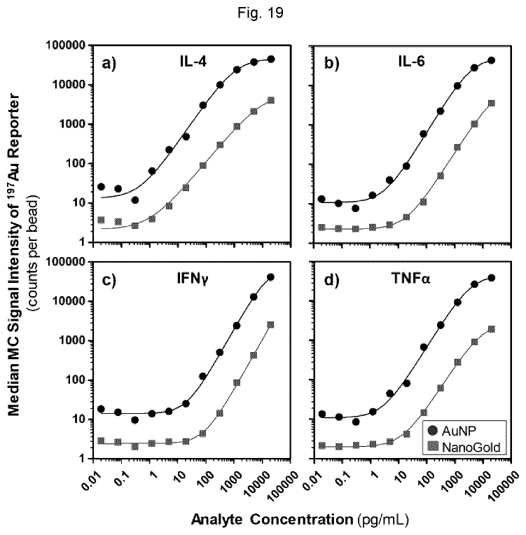

FIG. 19 summarizes the median MC signal intensities of different types of NPs attached to classifier beads at different analyte concentrations. In the low concentration region (up to ca. 1 pg/mL), the signal intensities of both types of reporters were low (˜10 counts per bead) and insensitive to the increase of analyte concentration. At higher analyte concentrations ranging from 1 to 1000 pg/mL, the signal intensities of these reporters increased significantly with the increase of analyte concentration. The signal intensities of AuNPs approached a plateau when the concentrations of analytes were above 1000 pg/mL, while curve of the assays using NanoGold® as the reporter showed similar trends but much lower intensities compared with that using AuNP as the reporter. Because of the much higher signal intensities were obtained at lower analyte concentrations in these assays, AuNPs were chosen as the reporter candidate for the subsequent bead-based assays in this study to optimize the lower limit of detection.

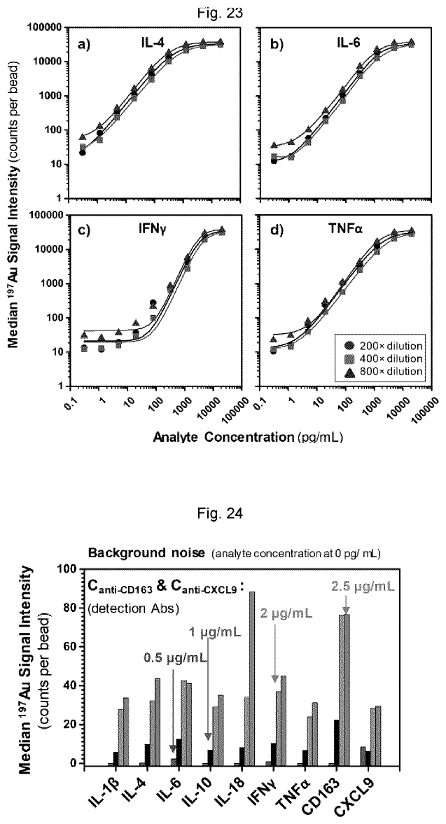

Optimization of the Reporter Concentration. The effect of NP concentration on assay signal intensity levels was then investigated. In this study, the stock AuNP dispersion was diluted 200, 400 and 800 times with 0.5% BSA buffer. These dilutions were then employed as reporter solutions in three sets of four-plex assays to analyze standard solutions. FIG. 23 shows the standard curves of the four-plex assays carried out under different AuNP concentrations. The signal intensities of AuNP at all three dilutions showed minimal differences at analyte concentrations above 20 pg/mL. The detection limits of assays stained with 200× diluted AuNP dispersion were as low as 0.3 pg/mL for IL-4, IL-6 and TNFα, while that for IFNγ was slightly higher at 1.2 pg/mL. 200× AuNP dilution was selected to use for the bead-based assays in this study.

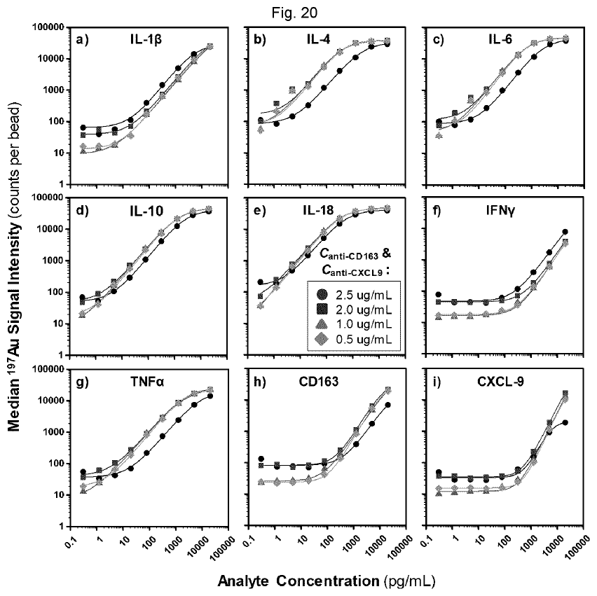

Optimization of Detection Ab Concentration (in Nine-Plex Assays). After using a series of four-plex assays as a proof-of-concept experiment, the multiplexing capacity of the assays was expanded to nine analytes by adding the analysis of IL-1β, IL-10, IL-18, CD163, and CXCL-9 to the panel. The experimental conditions developed in the four-plex assays were adapted to analyze a series of standard solutions containing nine analytes. In the first set of nine-plex assays, the concentration of each type of biotinylated Ab in the detection Ab cocktail was 2.5 μg/mL. The results are plotted as filled circles (●) in FIG. 20. The median intensity levels of AuNP signals on all types of classifier beads at low analyte concentrations (≤1.22 pg/mL) were observed to be higher (˜100 counts per bead) compared with the four-plex assays. Without wishing to be bound by theory, these relatively higher levels of signal intensities at low analyte concentrations could be attributed to background noise in the assays, possibly a consequence of non-specific binding.

To minimize the non-specific binding in the nine-plex assays, three sets of nine-plex assays were carried out, in which the concentrations of biotinylated anti-CD163 and anti-CXCL-9 were reduced to 2.0, 1.0 and 0.5 μg/mL, while the concentrations of other detection Abs remained at 2.5 μg/mL in the detection Ab cocktail. The concentrations of the detection anti-CD163 and anti-CXCL-9 were reduced in these assays, in part because the possible cross-reactivity and interference of detection Abs for CXCL-9 and CD163 with interleukins (ILs) at high Ab concentration may lead to more non-specific binding. The standard curves of these nine-plex assays were plotted in FIG. 20. For the assays of the standard solutions containing low concentrations (≤1.22 pg/mL) of the analytes, the median signal intensities in the assays that employed low concentrations (0.5 and 1.0 μg/mL) of biotinylated anti-CD163 and anti-CXCL-9 Abs were significantly lower than those obtained employing higher concentrations (2.0 and 2.5 μg/mL) of detection Abs. In the assays of the standard solutions with high analyte concentrations (≥1250 μg/mL), the signal intensity levels and assay sensitivities were similar under all of the experimental conditions. Overall, the detection sensitivities at low analyte concentrations and detection ranges for the analytes were improved, when the concentrations of biotinylated anti-CD163 and anti-CXCL-9 Abs in the detection Ab cocktails were lowered to 0.5 and 1.0 μg/mL. As shown in FIG. 24, the background noise in these nine-plex assays of blank solutions was also substantially reduced as the concentrations of biotinylated anti-CD163 and anti-CXCL-9 Abs decreased from 2.0 to 0.5 μg/mL. Based on these results, it seems that the non-specific binding of biotinylated anti-CD163 and anti-CXCL-9 to classifier beads can be minimized by reducing their detection Ab concentrations in the assays.

### Bead-Based Sandwich Immunoassays of Cytokines in Biological Samples

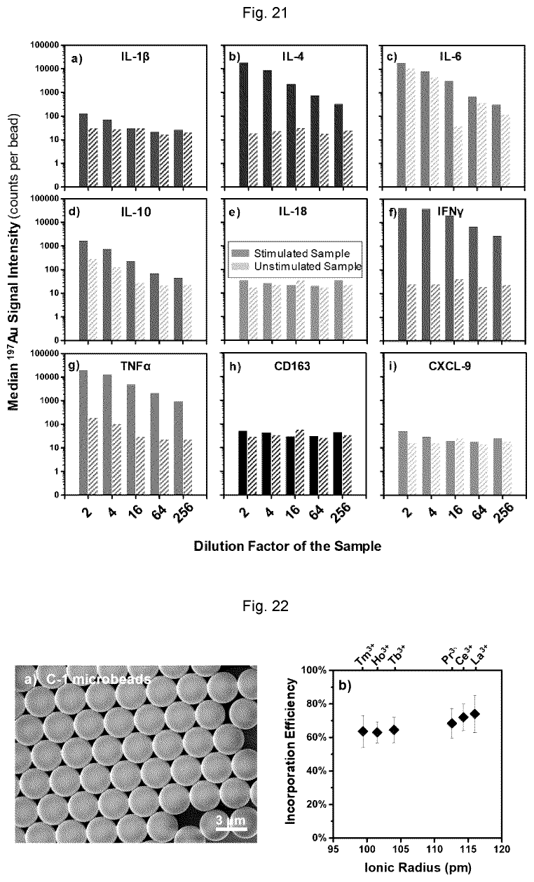

To examine the performance of the nine-plex assay on biological samples, a commercial PBMC sample (from a healthy donor) was used. One sample of the PBMCs suspended in cell culture media (RPMI) was stimulated with PMA/ionomycin, and analyzed for the extracellular release of cytokines in the supernatant of the stimulated PBMC suspension by the nine-plex assays using the above optimized assay conditions. PMA has a structure analogous to diacylglycerol and can diffuse through the cell membrane into the cytoplasm. In the cytoplasm, PMA activates protein kinase C. When used in combination with ionomycin, a calcium ionophore that triggers calcium release, a moderate level of cytokine is released from cells. An unstimulated sample was collected from the supernatant of an unstimulated PBMC suspension and analyzed as a control in this experiment. Prior to the assays, the stimulated sample and unstimulated sample were diluted 2×4×, 16×, 64× and 256× in the assays to vary the analyte concentrations in the measurements, so that some of the assays produce MC intensity values within the range of the standard curves. The median ¹⁹⁷Au signal intensities of AuNP attached to the classifier beads in these assays are presented in FIG. 21. In a typical assay, sample solution (50 μL) without dilution or diluted at the ratios of 1:2, 1:8, 1:32 or 1:128 was added to the classifier dispersion (50 μL). The detection Ab concentrations of anti-CD163 and anti-CXCL9 in the assays were 0.5 μg/mL and 1.0 μg/mL, while the concentrations of other detection Abs were 2.5 μg/mL. Substantially higher ¹⁹⁷ Au signals were detected from the IL-4, IFNγ and TNFα-classifier beads in the assays of the stimulated samples at all dilutions than from the unstimulated samples. This result is consistent with the fact that the stimulation process can lead to an elevation of the release of IL-4, IFNγ and TNFα by PBMCs, and is in general agreement with the findings reported by Ai et al.¹⁸ on samples stimulated by a similar process.

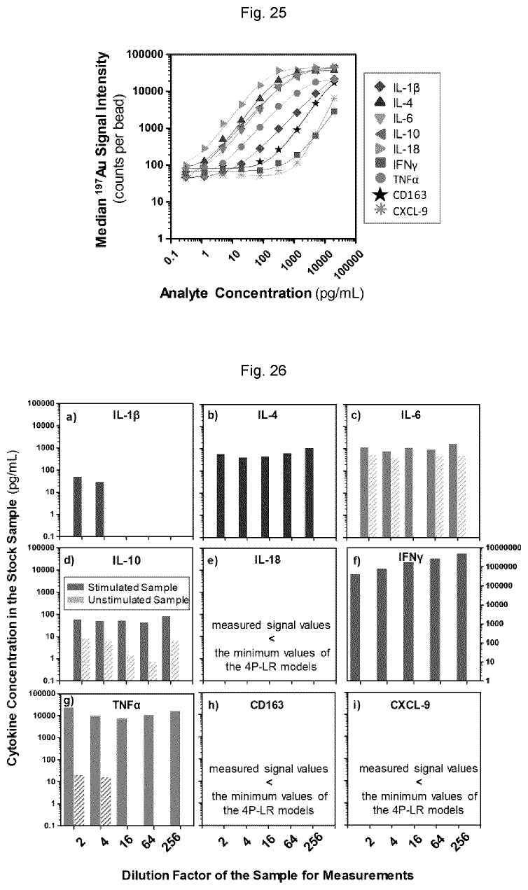

The same assay conditions were used to measure a series of standard solutions containing nine target cytokines with known concentrations. A series of standard curves were then developed by fitting the assay results of standard solutions with four-parameter logistic regression (4P-LR) models. (See FIG. 25) Using these standard curves the concentrations of these cytokines in the unstimulated and stimulated samples were assessed. However, some assay results in FIG. 21 were lower than the minimum values of the standard curves presented in FIG. 25. Therefore, no concentration could be concluded from these values. FIG. 26 summarizes the preliminary findings of the cytokine concentrations by fitting the assay results presented in FIG. 21 into the standard curves presented in FIG. 25. Since the dilution factors were considered in the calculation of the cytokine concentrations presented in FIG. 26, theoretically, similar concentration values for the same sample measured should be observed from different dilutions. For the measurement of IL-4 concentration in the stimulated sample, samples diluted with ratios of 1:1, 1:2, 1:8, 1:32, and 1:128 reported IL-4 concentrations of 600, 430, 460, 640, and 1110 pg/mL in the stock solution of stimulated sample, respectively, while for the unstimulated sample, none of the assays were able to produce enough signal intensities to assess the IL-4 concentrations, likely due to the low IL-4 concentration in the unstimulated sample. For the measurement of IFNγ concentration in the stimulated sample, the assays at different dilutions all reported very high signal intensities, which were higher than that of the most concentrated standard solution.

### CONCLUSION

A set of 11 types of lanthanide-encoded microbeads was synthesized by two-stage DisP. The metal content of the six metals in these microbeads was finely controlled by varying the feed of the metal complexes in the second stage of the DisP to produce microbeads generating signals in MC with median intensities of ca. 1000 counts per bead. These microbeads are uniform in size (CV_{diameter}<2%) and in metal content (RCV<15%), which makes them good candidates for classifier beads in bead-based assays.

In this bead-based assay by MC, cytokine levels were analyzed. To develop multiplexed bead-based sandwich immunoassays of cytokines and chemokines in MC, the surface of metal-encoded microbeads was modified with different types of Abs and employed as classifier beads to capture target analytes. To develop the reporter system and optimize assay conditions, a series of bead-based assays were first carried out to analyze a series of standard solutions containing up to nine types of cytokines and chemokines with known analyte concentrations. In the experiments, the MC signal intensities of reporter NPs were responsive to the concentration differences in the standard solutions. These assays showed high sensitivity at low analyte concentrations. However, non-specific binding of some of the detection Abs led to increased background noise issues. Background noise may compromise the detection limits of these assays. Further, the supernatants of two PBMC samples were analyzed by the nine-plex assays developed in this study for cytokines and chemokines. The assay results indicated that the supernatant of the PMA/ionomycin-stimulated sample contained elevated levels of IL-4, IFNγ and TNFα, compared with that of the unstimulated sample. This finding is in general agreement with results on cytokine detection reported in the literature measured by other assays. The results presented in this study show that bead-based sandwich immunoassays in MC can be used for simultaneous detection of different analytes.

### Example 10 Additional Experimental Details and Discussion of Example 9

### Materials

Styrene (St, Sigma-Aldrich, ≥99%), polyvinylpyrrolidone (PVP, Mw˜55 kDa), 2,2′-azobis(2-methylpropionitrile) (AIBN, 98%), Triton-X305 (TX305, 70% solution in water), diethylenetriaminepentaacetic dianhydride (DTPA dianhydride, 98%), phorbol 12-myristate 13-acetate (PMA), ionomycin, hydrogen peroxide solution (H₂O₂, 30% in H₂O), sulfuric acid (trace metal grade), and metal salts with the purity≥99.99% (trace metals basis), including lanthanum (III) chloride heptahydrate (LaCl₃·7H₂O), cerium (III) chloride heptahydrate (CeCl₃·7H₂O,), praseodymium (III) acetate hydrate [Pr(OAc)₃·xH₂O], terbium (III) chloride hexahydrate (TbCl₃·6H₂O), holmium (III) chloride hexahydrate (HoCl₃·6H₂O), and thulium (III) chloride hexahydrate (TmCl₃·6H₂O) were purchased from Millipore-Sigma. 4-Vinylbenzylamine (VBA, ≥92%) was purchased from TCI America. Absolute ethanol (EtOH) was purchased from Commercial Alcohols (Mississauga, Ontario). Single-element standard solutions for inductively coupled plasma mass spectrometry (ICP-MS) calibration were purchased from PerkinElmer (Pure Plus).

### Preparation of Metal-Encoded Classifier Beads

Metal complexes of DTPA-bis-vinylbenzyl amide (DTPA-VBAm₂) were employed as a chelator for incorporating different types of metal ions into polystyrene (PS) microbeads. The synthesis of the polymerizable DTPA-VBAm₂ metal chelator and the metal loading procedure are described in Example 4. To encode La, Ce, Pr, Tb and Tm into the microbeads, La (DTPA-VBAm₂), Ce (DTPA-VBAm₂), Pr (DTPA-VBAm₂), Tb (DTPA-VBAm₂) and Tm (DTPA-VBAm₂) were prepared in aqueous solution by mixing DTPA-VBAm₂ with LaCl₃, CeCl₃, Pr(OAc)₃, TbCl₃ and TmCl₃, respectively. These metal complexes were characterized by 1H NMR.

Microbeads as classifier beads for bead-based assays were synthesized by a series of two-stage dispersion polymerization (DisP) as described in Table 7. In a typical bead synthesis, the first stage of the polymerization of styrene (6.25 g) in absolute ethanol (18.75 g) was initiated by AIBN (0.25 g) at 70° C. in the presence of PVP (1 g) and TX305 (0.35 g) as stabilizers. The reaction was protected with N₂ purging (3 mL/min) controlled by a gas mass controller (OMEGA). 2 hours after the initiation of the reaction, a warm ethanol aliquot (15 g) containing different types of metal complexes of DTPA-VBAm₂ were added to the DisP reaction to incorporate metal ions into microbeads. The feed of M (DTPA-VBAm₂) in the second-stage aliquot was optimized to produce microbeads encoded with metal ions that generate a designed level of signal intensities in MC. (see Table 8) The reaction was terminated 24 h after the initiation and cooled to room temperature. Microbeads in the reaction dispersions were purified by two sedimentation-redispersion cycles with absolute ethanol and four cycles with water. Dispersions of purified microbeads were used for scanning electronic microscopy (SEM) imaging, MC measurements and further surface modification.

The diameters and diameter distributions of microbeads were characterized from their SEM images using a Hitachi S-5200 microscope. Typically, 2 μL of a diluted bead dispersion was dropped on a 300 mesh Formvar/carbon coated copper grid and allowed to dry. The diameters of microbeads were manually measured from multiple SEM images using ImageJ software. The mean average diameter, standard deviation (SD) and the coefficient of variation (CV) were calculated based on at least 300 measurements.

In the first two trails, the feed of metal complexes to the synthesis mixture was explored to obtain microbeads that can generate MC signals with intensities in the target range. These microbeads are denoted as Trial-1 and Trial-2. Based on the feed recipes developed in trial experiments, we then prepared 11 samples as classifier beads. These classifier beads are denoted C-1, C-2, . . . C-11, respectively, and are listed in Table.

### Acid Digestion of Microbead

After the termination of the DisP, an aliquot of the C-1 reaction dispersion was digested with H₂SO₄/H₂O₂ at 250° C. and analyzed by inductively coupled plasma mass spectrometry (ICP-MS) for the total metal content in the reaction. For the digestion of microbeads in the reaction dispersion, a microbead dispersion (100 μL, 0.5-2% solids content) and 500 μL of sulfuric acid were mixed in a 20 mL glass vial, heated to 250° C. on a hot plate and held for 40 min with magnetic stirring, followed by the addition of a 30% H₂O₂ solution (50 μL). For ICP-MS analysis, the digestion solution was subsequently diluted with 2% HNO₃. To quantify the free metal content in the reaction dispersion, the reaction stock dispersion was filtered through a syringe filter (0.2 μm, Nylon) to remove the microbeads and collected the filtrate for ICP-MS analysis. The metal incorporation efficiency in the C-1 reaction was estimated by comparing the total metal content with free metal content in the reaction mixture as described above in Example 4.

### Instrumentation

Inductively coupled plasma-mass spectrometry (ICP-MS). To quantify the metal content in samples, an ICP-MS (iCAP-Q, Thermo Scientific) system was employed. 2% HNO₃ was used to sequentially dilute the elemental standard solutions a series of concentrations of 40, 20, 10, 1, and 0.1 ppb as the calibration solutions. The metal content in each solution sample was determined based on the calibration fitting curves, with the detection limits below 10 ppt.

### Additional Results and Discussion

### Syntheses of Metal-Encoded Classifier Beads by Dispersion Polymerization

In this study, metal-encoded microbeads were prepared by two-stage DisP employing polymerizable metal complexes, M (DTPA-VBAm₂), to incorporate the metal ions into the PS microbeads. The synthesis of DTPA-VBAm₂ chelator was carried out in anhydrous DMSO by reacting DTPA dianhydride with 4-vinylbenzyl amine. La³⁺, Ce³⁺, Pr³⁺, Tb³⁺, Ho³⁺ and Tm³⁺ were loaded on the DTPA-VBAm₂ chelator in aqueous solution at pH 5˜6. The products of these syntheses were characterized by 1H-NMR. Details of the chelator synthesis and the metal load procedures are described in Example 4.

To develop a recipe for classifier bead synthesis, the step was to optimize the feed of M (DTPA-VBAm₂) in the second stage aliquot to prepare microbeads producing signals of ¹³⁹La, ¹⁴⁰Ce, ¹⁴¹Pr, ¹⁵⁹Tb, ¹⁶⁵Ho and ¹⁶⁹Tm in MC with intensity levels at 800˜1000 counts per bead for each of these isotopic channels. Based on the linear relationship between the metal feed and metal content is discussed herein in Example 4, some trial syntheses were carried out to design the feed of six metal complexes. Based on the feed recipes developed in the trial experiments, C-1 sample was prepared as a set of classifier beads encoded with six types of metal ions.

In addition, the metal incorporation efficiency of metal ions into C-1 beads was evaluated by ICP-MS. The results shown in panel (b) of FIG. 22 indicate that all six types of metal ions were effectively incorporated with efficiencies in the range of 63˜74%. The incorporation efficiency of each metal was evaluated by comparing the free metal content after the synthesis with the total metal content in the reaction. A slight increase in the efficiency was observed for the lanthanide metal ions with larger ionic radii, which agree with the findings described in above Example 4. Overall, C-1 beads qualify as a candidate classifier bead.

The size of microbeads prepared by DisP varies from batch to batch, possibly due to the sensitivity of particle nucleation to the reaction conditions. To optimize and prepare microbeads with small batch-to-batch variations in particle size, several control factors were explored in the experimental conditions and found that the reproducibility of particle sizes can be improved by keeping the N2 purging and reaction heating procedures consistent across batches. As summarized in Table 6, 11 types of microbeads (C-1 to C-11) were prepared that were uniform (CV<2%) and shared a similar size with their mean diameters in the range of 2.8 to 3.0 μm.

After the recipe optimization, additional types of classifier beads were prepared employing the feed of metal complexes developed for the C-1 synthesis. In the first set of samples, C-2 to C-6, two types of metal ions, Ce (DTPA-VBAm₂) plus a second type of metal complex were added in the bead syntheses. The amount of each metal complex in these di-metal bead syntheses were same as in the synthesis of the C-1 beads. The same principle was then applied to the syntheses of some tri-metal-encoded microbeads (C-7 to C11) as well.

TABLE 7
A typical recipe for the two-stage dispersion polymerization
of styrene with metal complex of DTPA-VBAm₂ derivatives

| materials (g) | 1^{st} stage ^{a} | 2^{nd} stage ^{b} |
|---|---|---|
| styrene | 6.25 | — |
| AIBN | 0.25 | — |
| PVP | 1.00 | — |
| TX305 | 0.35 | — |
| ethanol | 18.75 | 15.00 |
| DTPA-VBAm₂-metal | — | c |
| complexes | | |

^{a} The reaction was initiated by immersing the flask in a 70° C. oil bath. Prior to the initiation, the reaction solution was purged with nitrogen gas for 30 min.
^{b} The second-stage aliquot was introduced into the reaction mixture 2 h after the initiation.
c A desired amount of DTPA-VBAm₂-metal complexes [M(DTPA-VBAm₂)] dissolved in ethanol was introduced into the aliquot. Details of the metal addition are described in Table 8.

TABLE 8
Summary of the feed of M(DTPA-VBAm₂) in the second stage of
DisP for the synthesis of microbeads as the classifier beads

| | |
|---|---|
| Microbead | M(DTPA-VBAm₂) Complex Feed ^{a} (mg) |

| ID | La | Ce | Pr | Tb | Ho | Tm |
|---|---|---|---|---|---|---|
| C-1* | 7.6 | 8.2 | 7.6 | 3.7 | 3.3 | 3.8 |
| C-2* | 7.6 | 8.2 | — | — | — | — |
| C-3* | — | 8.2 | 7.6 | — | — | — |
| C-4* | — | 8.2 | — | 3.7 | — | — |
| C-5* | — | 8.2 | — | — | 3.3 | — |
| C-6* | — | 8.2 | — | — | — | 3.8 |
| C-7* | 7.6 | 8.2 | — | 3.7 | — | — |
| C-8* | — | 8.2 | 7.6 | 3.7 | — | — |
| C-9* | — | 8.2 | — | 3.7 | 3.3 | — |
| C-10* | — | 8.2 | — | 3.7 | — | 3.8 |
| C-11* | — | 8.2 | — | — | 3.3 | 3.8 |

^{a} Metal complexes of DTPA-VBAm₂ were first dissolved in ethanol (15 g) and then introduced to the DisP as the second stage aliquot.
* The “C” in sample notation stands for “classifier”.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### CITATIONS FOR REFERENCES REFERRED TO IN THE SPECIFICATION

(1) Kellar, K. L.; Iannone, M. A. Multiplexed Microsphere-Based Flow Cytometric Assays. Exp. Hematol. 2002, 30 (11), 1227-1237. https://doi.org/10.1016/S0301-472X (02) 00922-0.
(2) E. Gerver, R.; Gómez-Sjöberg, R.; C. Baxter, B.; S. Thorn, K.; M. Fordyce, P.; A. Diaz-Botia, C.; A. Helms, B.; L. DeRisi, J. Programmable Microfluidic Synthesis of Spectrally Encoded Microspheres. Lab. Chip 2012, 12 (22), 4716-4723.
(3) Chandler, D. J.; Lambert, B. A.; Reber, J. J.; Phipps, S. L. Precision Fluorescently Dyed Particles and Methods of Making and Using Same. U.S. Pat. No. 6,514,295B1, Feb. 4, 2003.
(4) Verkaik, N.; Brouwer, E.; Hooijkaas, H.; van Belkum, A.; van Wamel, W. Comparison of Carboxylated and Penta-His Microspheres for Semi-Quantitative Measurement of Antibody Responses to His-Tagged Proteins. J. Immunol. Methods 2008, 335 (1), 121-125.
(5) Bedre, J.; Chandler, D.; Mize, B. Method and System for Manufacture and Use of Macroporous Beads in a Multiplex Assay. U.S. Pat. No. 8,877,511B2, Nov. 4, 2014.
(6) Bandura, D. R.; Baranov, V. I.; Ornatsky, O. I.; Antonov, A.; Kinach, R.; Lou, X.; Pavlov, S.; Vorobiev, S.; Dick, J. E.; Tanner, S. D. Mass Cytometry: Technique for Real Time Single Cell Multitarget Immunoassay Based on Inductively Coupled Plasma Time-of-Flight Mass Spectrometry. Anal. Chem. 2009, 81 (16), 6813-6822.
(7) Bendall, S. C.; Nolan, G. P.; Roederer, M.; Chattopadhyay, P. K. A Deep Profiler's Guide to Cytometry. Trends Immunol. 2012, 33 (7), 323-332.
(8) Spitzer, M. H.; Nolan, G. P. Mass Cytometry: Single Cells, Many Features. Cell 2016, 165 (4), 780-791.
(9) Abdelrahman, A. I.; Dai, S.; Thickett, S. C.; Ornatsky, O.; Bandura, D.; Baranov, V.; Winnik, M. A. Lanthanide-Containing Polymer Microspheres by Multiple-Stage Dispersion Polymerization for Highly Multiplexed Bioassays. J. Am. Chem. Soc. 2009, 131 (42), 15276-15283.
(10) Abdelrahman, A. I.; Ornatsky, O.; Bandura, D.; Baranov, V.; Kinach, R.; Dai, S.; Thickett, S. C.; Tanner, S.; Winnik, M. A. Metal-Containing Polystyrene Beads as Standards for Mass Cytometry. J. Anal. At. Spectrom. 2010, 25 (3), 260-268.
(11) Liang, Y.; Abdelrahman, A. I.; Baranov, V.; Winnik, M. A. The Synthesis and Characterization of Lanthanide-Encoded Poly(Styrene-Co-Methacrylic Acid) Microspheres. Polymer 2011, 52 (22), 5040-5052.
(12) Thickett, S. C.; Abdelrahman, A. I.; Ornatsky, O.; Bandura, D.; Baranov, V.; Winnik, M. A. Bio-Functional, Lanthanide-Labeled Polymer Particles by Seeded Emulsion Polymerization and Their Characterization by Novel ICP-MS Detection. J. Anal. At. Spectrom. 2010, 25 (3), 269-281.
(13) Tan, J.; Zhao, G.; Zeng, Z.; Winnik, M. A. PMMA Microspheres with Embedded Lanthanide Nanoparticles by Photoinitiated Dispersion Polymerization with a Carboxy-Functional Macro-RAFT Agent. Macromolecules 2015, 48 (11), 3629-3640.
(14) Liang, Y.; Abdelrahman, A. I.; Baranov, V.; Winnik, M. A. The Release and Extraction of Lanthanide Ions from Metal-Encoded Poly(Styrene-Co-Methacrylic Acid) Microspheres. Polymer 2012, 53 (4), 998-1004.
(15) Liu, J.; Jarzabek, J.; Majonis, D.; Watson, J.; Baranov, V.; Winnik, M. A. Metal-Encoded Polystyrene Microbeads as a Mass Cytometry Calibration/Normalization Standard Covering Channels from Yttrium (89 Amu) to Bismuth (209 Amu). Anal. Chem. 2020, 92 (1), 999-1006.
(16) Zhang, P.; Guo, Z.; Zhang, D.; Liu, C.; Chen, G.; Zhuang, R.; Song, M.; Wu, H.; Zhang, X. A Novel Copolymer-Based Functional SPECT/MR Imaging Agent for Asialoglycoprotein Receptor Targeting. Mol. Imaging 2016, 15, 1536012116667327.
(17) Aime, S.; Benetollo, F.; Bombieri, G.; Colla, S.; Fasano, M.; Paoletti, S. Non-Ionic Ln (III) Chelates as MRI Contrast Agents: Synthesis, Characterisation and 1H NMR Relaxometric Investigations of Bis(Benzylamide) Diethylenetriaminepentaacetic Acid Lu(III) and Gd(III) Complexes. Inorganica Chim. Acta 1997, 254 (1), 63-70.
(18) Shuhendler, A. J.; Staruch, R.; Oakden, W.; Gordijo, C. R.; Rauth, A. M.; Stanisz, G. J.; Chopra, R.; Wu, X. Y. Thermally-Triggered ‘off-on-off’ Response of Gadolinium-Hydrogel-Lipid Hybrid Nanoparticles Defines a Customizable Temperature Window for Non-Invasive Magnetic Resonance Imaging Thermometry. J. Controlled Release 2012, 157 (3), 478-484.
(19) Song, J.-S.; Tronc, F.; Winnik, M. A. Two-Stage Dispersion Polymerization toward Monodisperse, Controlled Micrometer-Sized Copolymer Particles. J. Am. Chem. Soc. 2004, 126 (21), 6562-6563.
(20) Leipold, M. D.; Ornatsky, O.; Baranov, V.; Whitfield, C.; Nitz, M. Development of Mass Cytometry Methods for Bacterial Discrimination. Anal. Biochem. 2011, 419 (1), 1-8.
(21) Brown, H. M. G.; Arriaga, E. A. Quantifying Heterogeneity of Individual Organelles in Mixed Populations via Mass Cytometry. Anal. Chem. 2018, 90 (22), 13315-13321.
(22) Cacheris, W. P.; Quay, S. C.; Rocklage, S. M. The Relationship between Thermodynamics and the Toxicity of Gadolinium Complexes. Magn. Reson. Imaging 1990, 8 (4), 467-481.
(23) Smith, R. M.; Martell, A. E. Critical Stability Constants, Enthalpies and Entropies for the Formation of Metal Complexes of Aminopolycarboxylic Acids and Carboxylic Acids. Sci. Total Environ. 1987, 64 (1), 125-147.
(24) Paul-Roth, C.; Raymond, K. N. Amide Functional Group Contribution to the Stability of Gadolinium (III) Complexes: DTPA Derivatives. Inorg. Chem. 1995, 34 (6), 1408-1412.
(25) Bartlett, P. D.; Altschul, R. The Polymerization of Allyl Compounds. I. Factors Governing the Acyl Peroxide-Induced Polymerization of Allyl Acetate, and the Fate of the Peroxide. J. Am. Chem. Soc. 1945, 67 (5), 812-816.
(26) Matsumoto, A.; Kumagai, T.; Aota, H.; Kawasaki, H.; Arakawa, R. Reassessment of Free-Radical Polymerization Mechanism of Allyl Acetate Based on End-Group Determination of Resulting Oligomers by MALDI-TOF-MS Spectrometry. Polym. J. 2009, 41 (1), 26-33.
(27) Otsu, T.; Yamashita, K.; Tsuda, K. Synthesis, Reactivity, and Role of 4-Vinylbenzyl N,N-Diethyldithiocarbamate as a Monomer-Iniferter in Radical Polymerization. Macromolecules 1986, 19 (2), 287-290.
(28) Jin, L. Y.; Hou, R.; Chen, T.; Fang, M.; Mah, S.; Yin, B. Synthesis of N-4-Vinylbenzyl-Monoaza-m-Phenylene-34-Crown-11 and Its Copolymerization with Styrene. Fibers Polym. 2007, 8 (2), 143-147.
(29) Hazer, B.; Arslan, H.; Senemoğlu, Y.; Şen, Ş. Synthesis of Block/Graft Copolymers Based on Vinyl Benzyl Chloride via Reversible Addition Fragmentation Chain Transfer (RAFT) Polymerization Using the Carboxylic Acid Functionalized Trithiocarbonate. J. Polym. Res. 2019, 26 (5), 101.
(30) Hsieh, W.-Y.; Liu, S. Synthesis, Characterization, and Structures of Indium In (DTPA-BA2) and Yttrium Y (DTPA-BA2) (CH3OH) Complexes (BA=Benzylamine): Models for 111 In- and 90Y-Labeled DTPA-Biomolecule Conjugates. Inorg. Chem. 2004, 43 (19), 6006-6014.
(31) Song, J.-S.; Winnik, M. A. Monodisperse, Micrometer-Sized Low Molar Mass Polystyrene Particles by Two-Stage Dispersion Polymerization. Polymer 2006, 47 (13), 4557-4563.
(32) Liu, S.; Pietryka, J.; Ellars, C. E.; Edwards, D. S. Comparison of Yttrium and Indium Complexes of DOTA-BA and DOTA-MBA: Models for 90Y- and 111 In-Labeled DOTA-Biomolecule Conjugates. Bioconjug. Chem. 2002, 13 (4), 902-913.
(33) Abdelrahman, A. I. Lanthanide-Encoded Polystyrene Microspheres for Mass Cytometry-Based Bioassays. PhD Thesis, University of Toronto, Toronto, ON, 2012.
(34) Tang, Y.; Liu, J.; Zhang, D.; Xu, Z.; Ji, J.; Wen, C. Cytokine Storm in COVID-19: The Current Evidence and Treatment Strategies. Front. Immunol. 2020, 11, Article 1708.
(35) Ramesh, G.; MacLean, A. G.; Philipp, M. T. Cytokines and Chemokines at the Crossroads of Neuroinflammation, Neurodegeneration, and Neuropathic Pain. Mediators Inflamm. 2013, 2013, Article 480739.
(36) Price, C. P.; Newman, D. J. Principles and Practice of Immunoassay; Palgrave Macmillan: London, England, 1991.
(37) Surenaud, M.; Manier, C.; Richert, L.; Thiébaut, R.; Levy, Y.; Hue, S.; Lacabaratz, C. Optimization and Evaluation of Luminex Performance with Supernatants of Antigen-Stimulated Peripheral Blood Mononuclear Cells. BMC Immunol. 2016, 17 (1), 44.
(38) McHugh, T. M. Chapter 33 Flow Microsphere Immunoassay for the Quantitative and Simultaneous Detection of Multiple Soluble Analytes. Methods Cell Biol. 1994, 42, 575-595.
(39) Carson, R. T.; Vignali, D. A. A. Simultaneous Quantitation of 15 Cytokines Using a Multiplexed Flow Cytometric Assay. J. Immunol. Methods 1999, 227 (1), 41-52.
(40) Vignali, D. A. A. Multiplexed Particle-Based Flow Cytometric Assays. J. Immunol. Methods 2000, 243 (1), 243-255.
(41) Kellar, K. L.; Kalwar, R. R.; Dubois, K. A.; Crouse, D.; Chafin, W. D.; Kane, B.-E. Multiplexed Fluorescent Bead-Based Immunoassays for Quantitation of Human Cytokines in Serum and Culture Supernatants. Cytometry 2001, 45 (1), 27-36.
(42) Nolan, J. P.; Sklar, L. A. Suspension Array Technology: Evolution of the Flat-Array Paradigm. Trends Biotechnol. 2002, 20 (1), 9-12.
(43) Kellar, K. L.; Iannone, M. A. Multiplexed Microsphere-Based Flow Cytometric Assays. Exp. Hematol. 2002, 30 (11), 1227-1237.
(44) Harris, G.; Chen, W. Profiling of Cytokine and Chemokine Responses Using Multiplex Bead Array Technology. In Immunoproteomics: Methods and Protocols; Fulton, K. M., Twine, S. M., Eds.; Methods in Molecular Biology; Springer: New York, NY, 2019; pp 79-94.
(45) Knight, V.; Long, T.; Meng, Q. H.; Linden, M. A.; Rhoads, D. D. Variability in the Laboratory Measurement of Cytokines: A Longitudinal Summary of a College of American Pathologists Proficiency Testing Survey. Arch. Pathol. Lab. Med. 2020, 144 (10), 1230-1233.
(46) Liu, Jieyi. Synthesis and Modification of Metal-Encoded Microbeads for Applications in Mass Cytometry. PhD Thesis, University of Toronto, Toronto, Ontario, Canada, 2021.
(47) Guesdon, J. L.; Ternynck, T.; Avrameas, S. The Use of Avidin-Biotin Interaction in Immunoenzymatic Techniques. J. Histochem. Cytochem. 1979, 27 (8), 1131-1139.
(48) Dudley, R. A.; Edwards, P.; Ekins, R. P.; Finney, D. J.; Mckenzie, I. G.; Raab, G. M.; Rodbard, D.; Rodgers, R. P. Guidelines for Immunoassay Data Processing. Clin. Chem. 1985, 31 (8), 1264-1271.
(49) Ai, W.; Li, H.; Song, N.; Li, L.; Chen, H. Optimal Method to Stimulate Cytokine Production and Its Use in Immunotoxicity Assessment. Int. J. Environ. Res. Public. Health 2013, 10 (9), 3834-3842.

1. A metal-encoded microbead comprising:
a copolymer comprising:
a structural monomer, and
a metal-chelating monomer comprising a metal and a chelator;
wherein the chelator coordinates the metal at least at 3 sites; and
wherein the structural monomer does not comprise the chelator.

2. The microbead of claim 1, wherein the structural monomer is selected from substituted or unsubstituted styrene, alpha-methylstyrene, acrylic acid and esters and amides thereof, methacrylic acid and esters and amides thereof, and derivatives thereof, optionally the structural monomer is styrene.

3. The microbead of claim 1, wherein the metal-chelating monomer has a structure of Formula I prior to polymerization 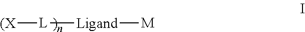
wherein Ligand is the chelator, L is a linker, X is a polymerizable end group, M is the metal, and n is 1 or an integer greater than 1, wherein the metal-chelating monomer is neutral in charge prior to polymerization,
optionally wherein L is selected from a bond, C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O)O, or mixtures thereof, optionally wherein each of the alkylene, aryl, alkylaryl, alkylheteroaryl, cycloalkyl, cycloalkylaryl, and cycloalkylheteroaryl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof, and/or
optionally wherein the polymerizable end group is selected from arylvinyl, styrene, alpha-methylstyrene, acrylate ester, methacrylate ester, acrylamide, 2-methylacrylamide, or mixtures thereof, optionally the polymerizable end group is arylvinyl or styrene.

4. The microbead of claim 3, wherein one or more of:
wherein L is attached to the chelator through an amide or an ester, and
wherein the chelator is tetradentate, pentadentate, hexadentate, heptadentate, or octadentate, optionally wherein the chelator is hexadentate or octadentate,
optionally wherein the chelator comprises an aminopolyacid moiety, or a derivative thereof, optionally wherein the aminopolyacid moiety is selected from aminopolycarboxylic acid, aminopolyphosphonic acid, or combinations thereof, optionally wherein the aminopolyacid moiety is a substituted oligomer of one or more of ethylene imine, propylene amine, or mixtures thereof, the oligomer being substituted with two or more carboxylic acids and/or phosphonic acids, optionally the oligomer is a crown ether or an aza-crown ether.

5. The microbead of claim 4, wherein the oligomer is further substituted with one or more substituents selected from C1-C6 alkyl, C1-C6 alkenyl, C3-C8 cycloalkyl, C3-C8 heterocycloalkyl, amide, ester, aryl, heteroaryl, akylaryl, alkylheteroaryl, C3-C8 cycloalkylaryl, C3-C8 cycloalkylheteroaryl, CN, or mixtures thereof.

6. The microbead of claim 4, wherein the chelator is selected from DFO, EDTA, DTPA, EGTA, EDDS, EDDHA, BAPTA, H₄neunpa, H₆phospa, H₄CHXoctapa, H₄octapa, H₂CHXdedpa, H₅decapa, Cy-DTPA, Ph-DTPA, a TACN-type chelator, a TACD-type chelator, a cyclen-type chelator, a cyclam-type chelator, a (13)aneN4-type chelator, a 1,7-diaza-12-crown-4-type chelator, a 1,10-diaza-18-crown-6-type chelator, or derivatives thereof.

7. The microbead of claim 6, wherein the TACN-type chelator is selected from NOTA, NOPO, TRAP, or derivatives thereof, wherein the cyclen-type chelator is selected from DOTA or derivatives thereof, wherein the cyclam-type chelator is selected from TETA, cross bridged-TETA, DiAmSar, or derivatives thereof, the (13)aneN4-type chelator is selected from TRITA or derivatives thereof, the 1,10-diaza-18-crown-6-type chelator is selected from MACROPA, or derivatives thereof, and/or wherein the chelator is selected from DTPA, Cy-DTPA, Ph-DTPA, or derivatives thereof.

8. The microbead of claim 3, wherein the metal-chelating monomer is selected from 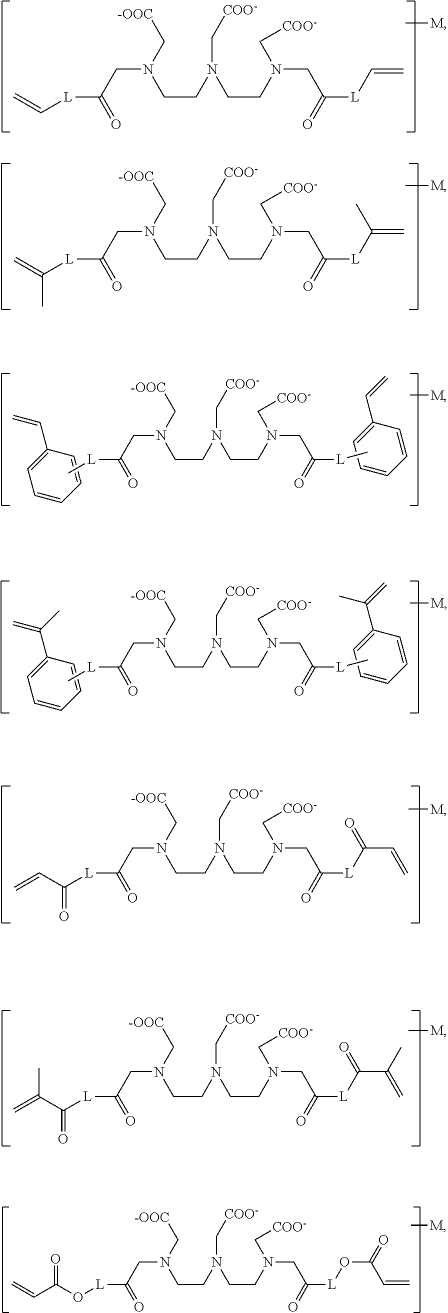 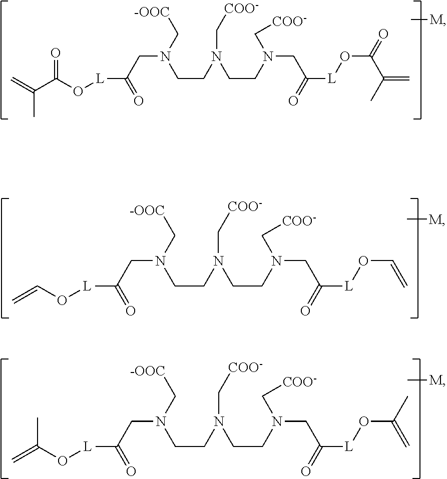
optionally wherein L is as defined in claim 3
optionally wherein the metal-chelating monomer is selected from 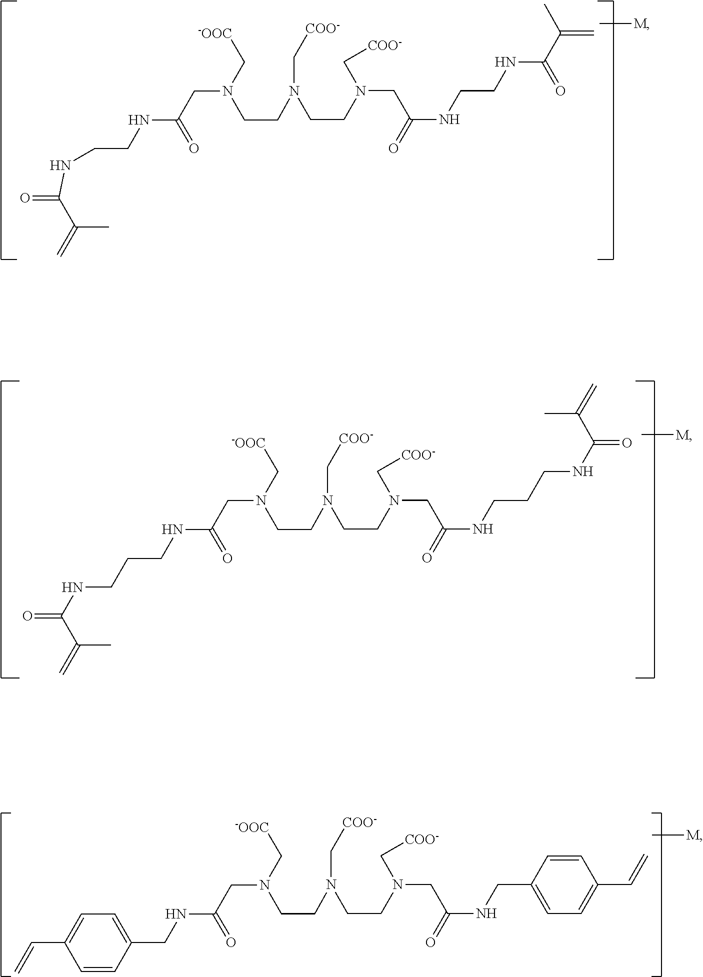
or mixtures thereof.

9. The microbead of claim 4, wherein the chelator comprises porphyrin or phthalocyanine, optionally the chelator is substituted or unsubstituted porphyrin, and
optionally wherein the metal-chelating monomer prior to polymerization is selected from 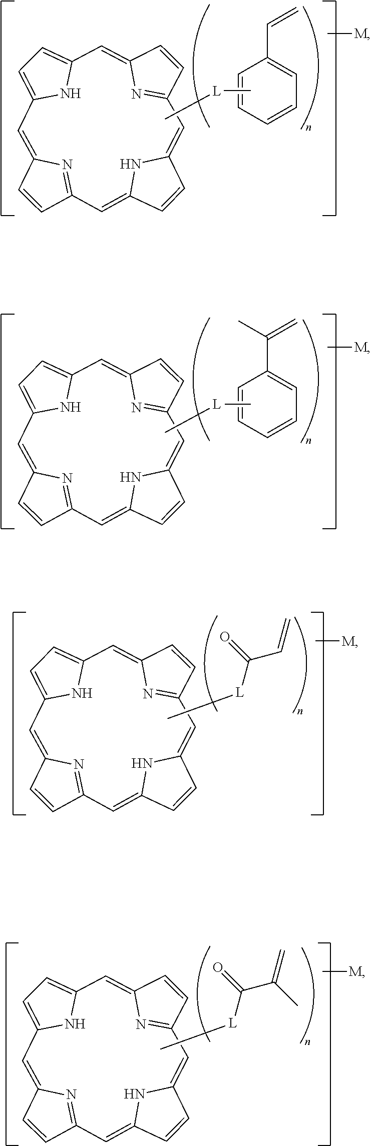
or mixtures thereof, and wherein n is an integer from 1 to 4, optionally wherein n is at least 2,
optionally wherein L is aniline.

10. The microbead of claim 1, wherein the metal is a plurality of metals,
optionally wherein one or more of:
wherein the plurality of metals comprises one or more enriched isotopes, optionally the plurality of metals comprises one or more enriched isotopes,
wherein the plurality of metals comprises at least 2 metals, at least 3 metals, or at least 4 metals,
wherein the amount of each metal of the plurality of metals is within about 20% or about 10% of the amount of another metal of the plurality of the metals, and
wherein the metal is distributed throughout the microbead, and
optionally wherein the metal comprises indium, bismuth, or a rare earth metal, optionally the rare earth metal is selected from lanthanide metal, yttrium, or mixtures thereof,
optionally the metal comprises a rare earth metal that is selected from Y, La, Ce, Pr, Nd, Pm, Sm, Eu, Gd, Tb, Dy, Ho, Er, Tm, Yb, Lu, distinct isotopes thereof, or mixtures thereof, and
optionally the rare earth metal is selected from 89Y, 139La, 136Ce, 138Ce, 140Ce, 142Ce, 141Pr, 142Nd, 143Nd, 145Nd, 146Nd, 148Nd, 145Pm, 144Sm, 149Sm, 150Sm, 152Sm, 154Sm, 151Eu, 153Eu, 154Gd, 155Gd, 156Gd, 157Gd, 158Gd, 160Gd, 152Gd, 159Tb, 156Dy, 158Dy, 160Dy, 161Dy, 162Dy, 163Dy, 164Dy, 165Ho, 162Er, 164Er, 166Er, 167Er, 168Er, 170Er, 169Tm, 168Yb, 170Yb, 171Yb, 172Yb, 173Yb, 174Yb, 176Yb, 175Lu, or mixtures thereof.

11. The microbead of claim 1, wherein one or more of:
wherein the microbead has a glass transition temperature of about 60° C. or above 60° C., optionally about 70° C. or above 70° C., about 80° C. or above 80° C., about 90° C. or above 90° C., about 100° C. or above 100° C., about 115° C. or above 115° C., about 125° C. or above 125° C., or about 135° C. or above 135° C., and
wherein the microbead has a diameter of about 0.6 μm to about 20 μm, about 1 μm to about 15 μm, about 2 μm to about 10 μm, about 2 μm to about 6 μm, and
wherein the microbead is colloidally stable in water.

12. The microbead of claim 1, wherein a surface of the microbead comprises functionalization for attachment to a biomolecule,
optionally wherein one or more of:
wherein the attachment is covalent attachment or non-covalent attachment,
wherein the surface of the microbead is functionalized with avidin, streptavidin, neutravidin, or mixtures thereof, and
wherein the surface of the microbead is conjugated to the biomolecule,
optionally wherein the biomolecule is selected from a protein, an oligonucleotide, a small molecule, a lipid, a carbohydrate, or a mixture thereof,
optionally the biomolecule is an affinity reagent, optionally wherein the affinity reagent is an antibody, optionally the antibody is specific for a cytokine, optionally a chemokine, an interferon, a lymphokine, a monokine, an interleukin, such as IL-1-36, tumor necrosis factor and colony stimulating factors, and optionally the antigen is a viral antigen, and
optionally wherein the functionalization comprises a coating of silicon dioxide on the surface of the microbead, optionally the functionalization further comprises functionalizing the coating of silicon dioxide.

13. The microbead of claim 12, wherein the metal provides a barcode that identifies the biomolecule.

14. A population of microbeads as defined in claim 1,
optionally wherein one or more of:
wherein the population has a size distribution having a coefficient of variation (CV) of about 10% or less than 10%, optionally the coefficient of variation is of less than 5%,
wherein each microbead comprises a plurality of metals, the average amount across the population of microbeads of each metal of the plurality of metals is about 10% or within 10% of the average amount of another metal of the plurality of metals,
optionally wherein the plurality of metals comprises one or more enriched isotopes,
wherein the amount of each metal of the population of microbeads has a distribution of a coefficient of variation of about 20% or less than 20%, or about 10% or less than 10%,
wherein the amount of each metal of one microbead of the population of microbeads is about 20% or within 20%, or about 10% or within 10%, or about 5% or within 5% of the amount of the same metal of another microbead of the population of microbeads, and
wherein the microbeads of the population of microbeads comprise the same metal in substantially the same amount, optionally the same metal is a plurality of metals and the microbeads comprise each metal of the plurality of metals in substantially the same amount.

15. (canceled)

16. (canceled)

17. A method of preparing a metal-encoded microbead comprising
polymerizing a structural monomer in the presence of a steric stabilizer in a nucleation stage to obtain a first mixture comprising polymerized structural monomer, unpolymerized structural monomer, and the steric stabilizer;
combining the first mixture with a metal-chelating monomer comprising a metal and a chelator attached to at least one polymerizable end group to obtain a second mixture,
wherein the chelator coordinates the metal at least at 3 sites and wherein the metal-chelating monomer is polymerizable with the structural monomer; and
polymerizing the second mixture to form a copolymer of the microbead;
wherein the structural monomer does not comprise the chelator.

18. The method of claim 17, wherein one or more of:
wherein the metal is a plurality of metals,
wherein the structural monomer is polymerized in the nucleation stage to about 5% to about 20% completion based on the structural monomer, and
wherein the polymerizing of the second mixture occurs to about 75% to about 100% completion, about 80% to about 99% completion, about 85% to about 95% completion, about 85% to about 93% completion based on the structural monomer.

19. The method of claim 17, the method further comprising functionalizing the microbead,
optionally wherein the functionalizing of the microbead comprises
mixing the polymerized second mixture with a third monomer to obtain a third mixture, the third monomer comprising a reactive functional group;
optionally the reactive functional group is selected from alcohol, aldehyde, carboxylic acid, epoxide, vinyl, alkyne, maleimide, or mixtures thereof, and
polymerizing the third mixture; and
optionally wherein one or more of:
wherein the functionalizing of the microbead comprises coating the microbead with silicon dioxide,
wherein the functionalizing of the microbead further comprises functionalizing the coating of silicon dioxide, and
wherein the method further comprising conjugating the microbead to a biomolecule.

20. A microbead prepared by the method of claim 17.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)
